(19) United States
(12) Patent Application Publication
(10) Pub. No.: US 2023/0392144 A1
(43) Pub. Date: Dec. 7, 2023

(54) COMPOSITIONS AND METHODS FOR REDUCING BASE CALL ERRORS BY REMOVING DEAMINATED NUCLEOTIDES FROM A NUCLEIC ACID LIBRARY

(71) Applicant: Element Biosciences, Inc., San Diego, CA (US)

(72) Inventors: Andrew PRICE, San Diego, CA (US); Junhua ZHAO, San Diego, CA (US); Semyon KRUGLYAK, San Diego, CA (US); Kelly BLEASE, San Diego, CA (US); Hua YU, San Diego, CA (US); Andrew ALTOMARE, San Diego, CA (US); Ryan KELLEY, San Diego, CA (US); Juan MORENO, San Diego, CA (US)

(21) Appl. No.: 18/328,650

(22) Filed: Jun. 2, 2023

Related U.S. Application Data
(60) Provisional application No. 63/349,016, filed on Jun. 3, 2022.

(51) Int. Cl. C12N 15/10; C12Q 1/6834

(52) U.S. Cl. CPC C12N 15/1093; C12Q 1/6834

(57) ABSTRACT

The present disclosure provides compositions comprising reagents employed in a nucleic acid library preparation workflow for removing deaminated bases, and methods for using the reagents. The compositions and methods described herein reduce base call errors, such as C:G to T:A transitions, in nucleic acid sequencing workflows.

### CROSS-REFERENCE TO RELATED APPLICATIONS

This application claims the benefit of and priority to U.S. Provisional Patent Application No. 63/349,016, filed Jun. 3, 2022, the contents of which are incorporated by reference herein in their entirety.

### REFERENCE TO AN ELECTRONIC SEQUENCE LISTING

The contents of the electronic sequence listing (ELEM_011_001US_SeqList_ST26.xml; Size: 58,832 bytes; and Date of Creation: Jun. 1, 2023) are herein incorporated by reference in its entirety.

### TECHNICAL FIELD

The present disclosure provides compositions comprising reagents employed in a nucleic acid library preparation workflow for removing deaminated bases, and methods for using the reagents. The compositions and methods described herein reduce base call errors, such as C:G to T:A transitions, in nucleic acid sequencing workflows.

### BACKGROUND

Polynucleotide sequencing technology has applications in biomedical research and healthcare settings. Improved methods of polynucleotide require enhanced surface chemistry, on-support polynucleotide amplification, and base calling. Currently, these elements produce barriers in existing sequencing technology that result in limits in throughput and poor signal-to-noise ratio, and ultimately to increased costs associated with polynucleotide sequencing.

There exists a need for new polynucleotide sequencing methods with improved surface chemistry, on-support amplification, and base calling. The present disclosure provides methods and compositions to improve sequencing of polynucleotides by improving base-calling and subsequently increasing sequencing quality.

### SUMMARY

In one aspect, the present disclosure provides a method for reducing deaminated nucleotide bases in a nucleic acid library, comprising: providing a plurality of linear nucleic acid library molecules, wherein individual library molecules in the plurality comprise a sequence of interest joined to at least one universal adaptor sequence having a binding sequence for a surface capture primer and one universal adaptor sequence having a binding sequence for a sequencing primer, and wherein at least one of the library molecule carries one or more deaminated nucleotide bases; contacting the plurality of nucleic acid library molecules with a reagent that removes deaminated nucleotide bases, thereby generating at least one library molecule carrying an abasic site; circularizing the plurality of nucleic acid library molecules to generate a plurality of covalently closed circular library molecules; distributing the plurality of covalently closed circular library molecules onto a support having a plurality of immobilized surface capture primers, under a condition suitable for hybridizing individual covalently closed circular library molecules to a surface capture primer; conducting a rolling circle amplification reaction to generate a plurality of nucleic acid concatemer template molecules immobilized to the support; and sequencing the plurality of nucleic acid concatemer template molecules to determine the sequence of at least a portion of the concatemer template molecules.

In some embodiments, the method further comprises (g) contacting the plurality of covalently closed circular library molecules with a reagent the removes deaminated nucleotide bases, thereby generating at least one circular library molecule carrying an abasic site.

In some embodiments, the reagent that removes deaminated nucleotide bases of step (b) comprises DNA glycosylase (UDG) and (i) AP lyase, (ii) Endo IV endonuclease, (iii) FPG glycosylase/AP lyase, and/or (iv) Endo VIII glycosylase/AP lyase, or a combination thereof.

In some embodiments, the reagent that removes deaminated nucleotide bases of step (g) comprises DNA glycosylase (UDG) and (i) AP lyase, (ii) Endo IV endonuclease, (iii) FPG glycosylase/AP lyase, and/or (iv) Endo VIII glycosylase/AP lyase, or a combination thereof.

In some embodiments, the plurality of immobilized surface capture primers is tethered to a polymer coating on the support.

In some embodiments, the rolling circle amplification reaction of step (e) comprises a strand displacing polymerase and a plurality of nucleotides comprising dATP, dGTP, dCTP, dTTP and/or dUTP.

In some embodiments, the plurality of immobilized surface capture primers is located at pre-determined locations on the support.

In some embodiments, the plurality of immobilized surface capture primers is located at random locations on the support.

In some embodiments, the plurality of immobilized concatemer template molecules on the support is in fluid communication with each other to permit flowing a solution of reagents onto the support. In some embodiments, the solution of reagents comprises enzymes, nucleotides, and divalent cations.

In some embodiments, the plurality of immobilized concatemer template molecules is essentially simultaneously reacted with the reagents in a massively parallel manner.

In some embodiments, the density of the plurality of immobilized concatemer template molecules on the polymer-coated support is 10²-10¹² per mm².

In some embodiments, sequencing the plurality of immobilized concatemers comprises: contacting the plurality of immobilized concatemer molecules with (i) a plurality of sequencing polymerases and (ii) a plurality of soluble sequencing primers, wherein the contacting is conducted under a condition suitable to form a plurality of complexed polymerases each comprising a sequencing polymerase bound to a nucleic acid duplex, wherein the nucleic acid duplex comprises a concatemer molecule hybridized to a soluble sequencing primer; contacting the plurality of complexed sequencing polymerases with a plurality of nucleotides under a condition suitable for binding at least one nucleotide to a complexed sequencing polymerase, wherein the plurality of nucleotides comprises at least one nucleotide analog labeled with a fluorophore and having a removable chain terminating moiety at the sugar 3′ position; incorporating at least one nucleotide into the 3′ end of the hybridized sequencing primers, thereby generating a plurality of nascent extended sequencing primers; and detecting the incorporated nucleotide and identifying the nucleo-base of the incorporated nucleotide.

In some embodiments, the plurality of nucleotides comprises a removable chain terminating moiety at the 3′ sugar group, wherein the removable chain terminating moiety comprises an alkyl group, alkenyl group, alkynyl group, allyl group, aryl group, benzyl group, azide group, azido group, O-azidomethyl group, amine group, amide group, keto group, isocyanate group, phosphate group, thio group, disulfide group, carbonate group, urea group, or silyl group.

In some embodiments, the removable chain terminating moiety is cleavable with a chemical compound to generate an extendible 3′OH moiety on the sugar group.

In some embodiments, the plurality of nucleotides comprises one type of nucleotide selected from the group consisting of dATP, dGTP, dCTP, dTTP and dUTP.

In some embodiments, the plurality of nucleotides comprises a mixture of any combination of two or more types of nucleotides selected from the group consisting of dATP, dGTP, dCTP, dTTP and dUTP.

In some embodiments, sequencing the plurality of immobilized concatemers comprises: contacting the plurality of immobilized concatemer molecules with (i) a plurality of sequencing polymerases and (ii) a plurality of the soluble sequencing primers, wherein the contacting is conducted under a condition suitable to form a plurality of first complexed polymerases each comprising a sequencing polymerase bound to a nucleic acid duplex, wherein the nucleic acid duplex comprises a concatemer molecule hybridized to a soluble sequencing primer; contacting the plurality of complexed sequencing polymerases with a plurality of detectably labeled multivalent molecules to form a plurality of multivalent-complexed polymerases, under a condition suitable for binding complementary nucleotide units of the multivalent molecules to at least two of the plurality of first complexed polymerases thereby forming a plurality of multivalent-complexed polymerases, and the condition inhibits incorporation of the complementary nucleotide units into the sequencing primers of the plurality of multivalent-complexed polymerases, wherein individual multivalent molecules in the plurality of multivalent molecules comprise a core attached to multiple nucleotide arms and each nucleotide arm is attached to a nucleotide unit; detecting the plurality of multivalent-complexed polymerases; and identifying the nucleo-base of the complementary nucleotide units that are bound to the plurality of first complexed polymerases in the plurality of multivalent-complexed polymerases, thereby determining the sequence of the nucleic acid template.

In some embodiments, the method further comprises dissociating the plurality of multivalent-complexed polymerases, removing the plurality of first sequencing polymerases and their bound multivalent molecules, and retaining the plurality of nucleic acid duplexes; contacting the plurality of the retained nucleic acid duplexes of step (e) with a plurality of second sequencing polymerases, wherein the contacting is conducted under a condition suitable for binding the plurality of second sequencing polymerases to the plurality of the retained nucleic acid duplexes, thereby forming a plurality of second complexed polymerases each comprising a second sequencing polymerase bound to a retained nucleic acid duplex; contacting the plurality of second complexed polymerases with a plurality of nucleotides comprising at least one nucleotide analog labeled with a fluorophore and having a removable chain terminating moiety at the sugar 3′ position, wherein the contacting is conducted under a condition suitable for binding complementary nucleotides from the plurality of nucleotides to at least two of the second complexed polymerases of step (f) thereby forming a plurality of nucleotide-complexed polymerases and the condition is suitable for promoting incorporation of the bound complementary nucleotides into the sequencing primers of the nucleotide-complexed polymerases.

In some embodiments, the method further comprises detecting the complementary nucleotides which are incorporated into the sequencing primers of the nucleotide-complexed polymerases.

In some embodiments, the method further comprises detecting the complementary nucleotides which are incorporated into the sequencing primers of the nucleotide-complexed polymerases; and identifying the nucleo-bases of the complementary nucleotides which are incorporated into the sequencing primers of the nucleotide-complexed polymerases.

In another aspect, provided herein is a method for sequencing by forming at least one avidity complex, comprising: generating a nucleic acid concatemer by conducting a rolling circle amplification on a closed circular nucleic acid molecule comprising at least one abasic site, wherein the abasic site is generated by contacting a closed circular nucleic acid molecule or the corresponding linear nucleic acid molecule with a reagent that removes deaminated nucleotide bases; binding a first universal sequencing primer, a first sequencing polymerase, and a first detectably labeled multivalent molecule to a first portion of the concatemer molecule, thereby forming a first binding complex, wherein a first nucleotide unit of the first multivalent molecule binds to the first sequencing polymerase; binding a second universal sequencing primer, a second sequencing polymerase, and the first detectably labeled multivalent molecule to a second portion of the same concatemer molecule thereby forming a second binding complex, wherein a second nucleotide unit of the first multivalent molecule binds to the second sequencing polymerase, wherein the first and second binding complexes which include the same multivalent molecule forms an avidity complex, wherein the first detectably labeled multivalent molecule comprises a core attached to multiple nucleotide arms and each nucleotide arm is attached to a nucleotide unit, wherein the concatemer molecule comprises two or more tandem repeat sequences of a sequence of interest (110) and a universal primer binding site that binds the first and second universal sequencing primers, and wherein the contacting is conducted under a condition suitable to inhibit polymerase-catalyzed incorporation of the bound first and second nucleotide units in the first and second binding complexes; detecting the first and second binding complexes on the same concatemer molecule, and identifying the first nucleotide unit in the first binding complex thereby determining the sequence of the first portion of the concatemer template molecule, and identifying the second nucleotide unit in the second binding complex thereby determining the sequence of the second portion of the concatemer template molecule.

In some embodiments, the plurality of nucleotide arms attached to the core of the individual multivalent molecules has the same type of a nucleotide unit, and wherein the type of nucleotide unit is selected from the group consisting of dATP, dGTP, dCTP, dTTP and dUTP.

In some embodiments, the plurality of multivalent molecules comprises a mixture of two or more types of multivalent molecules, each type having a nucleotide unit selected from the group consisting of dATP, dGTP, dCTP, dTTP and dUTP.

In some embodiments, the plurality of nucleotides comprises a removable chain terminating moiety at the 3′ sugar group, wherein the removable chain terminating moiety comprises an alkyl group, alkenyl group, alkynyl group, allyl group, aryl group, benzyl group, azide group, azido group, O-azidomethyl group, amine group, amide group, keto group, isocyanate group, phosphate group, thio group, disulfide group, carbonate group, urea group, or silyl group.

In some embodiments, the removable chain terminating moiety is cleavable with a chemical compound to generate an extendible 3′OH moiety on the sugar group.

In some embodiments, the plurality of nucleotides comprises one type of nucleotide selected from the group consisting of dATP, dGTP, dCTP, dTTP and dUTP.

In some embodiments, the plurality of nucleotides comprises a mixture of any combination of two or more types of nucleotides selected from the group consisting of dATP, dGTP, dCTP, dTTP and dUTP.

In some embodiments, the support comprises a glass substrate.

In some embodiments, the support comprises a plastic substrate.

In some embodiments, the support is passivated with at least one hydrophilic polymer coating having a water contact angle of no more than 45 degrees. In some embodiments, the at least one hydrophilic polymer coating comprises a molecule selected from the group consisting of polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxylethyl methacrylate) (PHEMA), poly(oligo(ethylene glycol) methyl ether methacrylate) (POEGMA), polyglutamic acid (PGA), poly-lysine, poly-glucoside, streptavidin, and dextran.

In some embodiments, the method further comprises determining the percent base call error from the sequencing of step (f).

In some embodiments, the method further comprises determining the quality score of the sequencing data from the percent base call error. In some embodiments, the quality score is a Phred quality score.

### DESCRIPTION OF THE DRAWINGS

The features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a schematic showing an exemplary circularization of a linear single stranded library molecule (100) by hybridizing to a double-stranded splint molecule adaptor (200).

FIG. 2 is a schematic showing an exemplary library-splint complex (500) undergoing a ligation reaction to close the nicks to form a covalently closed circular library molecule (600) which is hybridized to a first splint strand (300), where the first splint strand (300) can be used as an amplification primer to conduct a rolling circle amplification reaction. The dotted line represents the nascent extension product.

FIG. 3 shows the nucleotide sequences of an exemplary double-stranded splint molecule (200) having a first splint strand (300) and a second splint strand (400). The exemplary first splint strand comprises a first region (320; SEQ ID NO:4), a second region (330; SEQ ID NO:5), and an internal region (310) having a fourth sub-region (SEQ ID NO:6) and fifth sub-region (SEQ ID NO:7). The first splint strand (300) comprises the first and second regions ((320) and (330)) and the internal region (310) (SEQ ID NO:8). The exemplary second splint strand (400) comprises a first sub-region (SEQ ID NO:1) and second sub-region (SEQ ID NO:2). The second splint strand (400) comprises the first and second sub-regions (SEQ ID NO:3).

FIG. 4 is a schematic showing an exemplary linear single stranded library molecule (700) hybridizing with a single-stranded splint molecule/strand (800) thereby circularizing the library molecule to form a library-splint complex (900) with a nick. The exemplary library molecule (700) comprises: a first left universal adaptor sequence (720); an optional first left unique identification sequence (780); a first left index sequence (760); a second left universal adaptor sequence (740); a sequence of interest (710); a second right universal adaptor sequence (750); a first right index sequence (770); and a first right universal adaptor sequence (730). The single-stranded splint strand (800) comprises a first region (810) that hybridizes with a sequence on one end of the linear single stranded library molecule, and a second region (820) that hybridizes with a sequence on the other end of the linear single stranded library molecule.

FIG. 5 is a schematic showing an exemplary library-splint complex (900) undergoing a ligation reaction to close the nick to form a covalently closed circular library molecule (1000) which is hybridized to a single-stranded splint strand (800), where the single-stranded splint strand (800) is used as an amplification primer to conduct a rolling circle amplification reaction. The dotted line represents the nascent extension product.

FIG. 6 shows the nucleotide sequences of an exemplary single-stranded splint molecule/strand (800; SEQ ID NO:203). The exemplary single-stranded splint strand comprises a first region (810; SEQ ID NO:9), a second region (820; SEQ ID NO:10).

FIG. 7 is Table 1 (3 sheets) which lists the sequences of exemplary first left index sequences (160) or (760), and Table 2 (3 sheets) which lists the sequences of exemplary first right index sequences (170) or (770) having a short random sequence (e.g., NNN).

FIG. 8 is a schematic of an exemplary low binding support comprising a glass substrate and alternating layers of hydrophilic coatings which are covalently or non-covalently adhered to the glass, and which further comprises chemically reactive functional groups that serve as attachment sites for oligonucleotide primers (e.g., capture oligonucleotides). In an alternative embodiment, the support can be made of any material such as glass, plastic, or a polymer material.

FIG. 9 is a schematic of various exemplary configurations of multivalent molecules. Left (Class I): schematics of multivalent molecules having a “starburst” or “helter-skelter” configuration. Center (Class II): a schematic of a multivalent molecule having a dendrimer configuration. Right (Class III): a schematic of multiple multivalent molecules formed by reacting streptavidin with 4-arm or 8-arm PEG-NHS with biotin and dNTPs. Nucleotide units are designated ‘N’, biotin is designated ‘B’, and streptavidin is designated ‘SA’.

FIG. 10 is a schematic of an exemplary multivalent molecule comprising a generic core attached to a plurality of nucleotide-arms.

FIG. 11 is a schematic of an exemplary multivalent molecule comprising a dendrimer core attached to a plurality of nucleotide-arms.

FIG. 12 shows a schematic of an exemplary multivalent molecule comprising a core attached to a plurality of nucleotide-arms, where the nucleotide arms comprise biotin, spacer, linker, and a nucleotide unit.

FIG. 13 is a schematic of an exemplary nucleotide-arm comprising a core attachment moiety, spacer, linker, and nucleotide unit.

FIG. 14 shows the chemical structure of an exemplary spacer (top), and the chemical structures of various exemplary linkers, including an 11-atom Linker, 16-atom Linker, 23-atom Linker, and an N3 Linker (bottom).

FIG. 15 shows the chemical structures of various exemplary linkers, including Linkers 1-9.

FIG. 16 shows the chemical structures of various exemplary linkers joined/attached to nucleotide units.

FIG. 17 shows the chemical structures of various exemplary linkers joined/attached to nucleotide units.

FIG. 18 shows the chemical structures of various exemplary linkers joined/attached to nucleotide units.

FIG. 19 shows the chemical structure of an exemplary linkers joined/attached to nucleotide units.

FIG. 20 shows the chemical structure of an exemplary biotinylated nucleotide-arm. In this example, the nucleotide unit is connected to the linker via a propargyl amine attachment at the 5 position of a pyrimidine base or the 7 position of a purine base.

FIG. 21 shows the chemical structures of various nucleotide bases and their corresponding deaminated bases, including: deamination of cytosine to uracil; deamination of adenine to hypoxanthine; deamination of guanine to xanthine; and deamination of 5-methylcytosine to thymine.

FIG. 22 is two graphs showing sequencing quality scores of concatemer template molecules generated by a workflow including circularizing linear library molecules using double-stranded splint adaptors (e.g., see FIGS. 1-3), with or without USER™ treatment and on-support rolling circle amplification to generate concatemer template molecules immobilized to a coated support. The control graph on the left shows the sequencing quality scores of base calls T, C, A and G of concatemer template molecules that generated with no USER™ treatment during the library prep workflow.

FIG. 23 is two graphs showing sequencing quality scores of concatemer template molecules generated by a workflow including circularizing linear library molecules using double-stranded splint adaptors (e.g., see FIGS. 1-3), with or without USER™ treatment and on-support rolling circle amplification to generate concatemer template molecules immobilized to a coated support. The control graph on the left shows the sequencing quality scores of base calls T, C, A and G of concatemer template molecules that generated with no USER™ treatment during the library prep workflow.

### DETAILED DESCRIPTION

### Definitions

The headings provided herein are not limitations of the various aspects of the disclosure, which aspects can be understood by reference to the specification as a whole.

Unless defined otherwise, technical and scientific terms used herein have meanings that are commonly understood by those of ordinary skill in the art unless defined otherwise. Generally, terminologies pertaining to techniques of molecular biology, nucleic acid chemistry, protein chemistry, genetics, microbiology, transgenic cell production, and hybridization described herein are those well-known and commonly used in the art. Techniques and procedures described herein are generally performed according to conventional methods well known in the art and as described in various general and more specific references that are cited and discussed throughout the instant specification. For example, see Sambrook et al., Molecular Cloning: A Laboratory Manual (Third ed., Cold Spring Harbor Laboratory Press, Cold Spring Harbor, N.Y. 2000). See also Ausubel et al., Current Protocols in Molecular Biology, Greene Publishing Associates (1992). The nomenclatures utilized in connection with, and the laboratory procedures and techniques described herein are those well-known and commonly used in the art.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative term (e.g., “or”) is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include: “A and B”; “A or B”; “A” (A alone); and “B” (B alone). In a similar manner, the term “and/or” as used in a phrase such as “A, B, and/or C” is intended to encompass each of the following aspects: “A, B, and C”; “A, B, or C”; “A or C”; “A or B”; “B or C”; “A and B”; “B and C”; “A and C”; “A” (A alone); “B” (B alone); and “C” (C alone).

As used herein and in the appended claims, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the terms “about” and “approximately” refer to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition. Also, where ranges and/or subranges of values are provided, the ranges and/or subranges can include the endpoints of the ranges and/or subranges.

The terms “peptide”, “polypeptide” and “protein” and other related terms used herein are used interchangeably and refer to a polymer of amino acids and are not limited to any particular length. Polypeptides may comprise natural and non-natural amino acids. Polypeptides include recombinant or chemically synthesized forms. Polypeptides also include precursor molecules that have not yet been subjected to post-translation modification such as proteolytic cleavage, cleavage due to ribosomal skipping, hydroxylation, methylation, lipidation, acetylation, SUMOylation, ubiquitination, glycosylation, phosphorylation and/or disulfide bond formation. These terms encompass native and artificial proteins, protein fragments and polypeptide analogs (such as muteins, variants, chimeric proteins, and fusion proteins) of a protein sequence as well as post-translationally, or otherwise covalently or non-covalently, modified proteins.

The term “cellular biological sample” refers to a single cell, a plurality of cells, a tissue, an organ, an organism, or section of any of these cellular biological samples. The cellular biological sample can be extracted (e.g., biopsied) from an organism, or obtained from a cell culture grown in liquid or in a culture dish. The cellular biological sample comprises a sample that is fresh, frozen, fresh frozen, or archived (e.g., formalin-fixed paraffin-embedded; FFPE). The cellular biological sample can be embedded in a wax, resin, epoxy or agar. The cellular biological sample can be fixed, for example in any one or any combination of two or more of acetone, ethanol, methanol, formaldehyde, paraformaldehyde-Triton, or glutaraldehyde. The cellular biological sample can be sectioned or non-sectioned. The cellular biological sample can be stained, de-stained or non-stained.

The nucleic acids of interest can be extracted from cells or cellular biological samples using any of a number of techniques known to those of skill in the art. For example, a typical DNA extraction procedure comprises (i) collection of the cell sample or tissue sample from which DNA is to be extracted, (ii) disruption of cell membranes (i.e., cell lysis) to release DNA and other cytoplasmic components, (iii) treatment of the lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis. A variety of suitable commercial nucleic acid extraction and purification kits are consistent with the disclosure herein. Examples include, but are not limited to, the QIAamp™ kits (for isolation of genomic DNA from human samples) and DNAeasy™ kits (for isolation of genomic DNA from animal or plant samples) from Qiagen™ (Germantown, MD), or the Maxwell® and ReliaPrep™ series of kits from Promega™ (Madison, WI).

The term “polymerase” and its variants, as used herein, comprises an enzyme comprising a domain that binds a nucleotide (or nucleoside) where the polymerase can form a complex having a template nucleic acid and a complementary nucleotide. The polymerase can have one or more activities including, but not limited to, base analog detection activities, DNA polymerization activity, reverse transcriptase activity, DNA binding, strand displacement activity, and nucleotide binding and recognition. A polymerase can be any enzyme that can catalyze polymerization of nucleotides (including analogs thereof) into a nucleic acid strand. Typically, but not necessarily, such nucleotide polymerization can occur in a template-dependent fashion. Typically, a polymerase comprises one or more active sites at which nucleotide binding and/or catalysis of nucleotide polymerization can occur. In some embodiments, a polymerase includes other enzymatic activities, such as for example, 3′ to 5′ exonuclease activity or 5′ to 3′ exonuclease activity. In some embodiments, a polymerase has strand displacing activity. A polymerase can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives, or fragments thereof that retain the ability to catalyze nucleotide polymerization (e.g., catalytically active fragment). The polymerase includes catalytically inactive polymerases, catalytically active polymerases, reverse transcriptases, and other enzymes comprising a nucleotide binding domain. In some embodiments, a polymerase can be isolated from a cell, or generated using recombinant DNA technology or chemical synthesis methods. In some embodiments, a polymerase can be expressed in prokaryote, eukaryote, viral, or phage organisms. In some embodiments, a polymerase can be post-translationally modified proteins or fragments thereof. A polymerase can be derived from a prokaryote, eukaryote, virus, or phage. A polymerase comprises DNA-directed DNA polymerase and RNA-directed DNA polymerase.

The term “strand displacing” refers to the ability of a polymerase to locally separate strands of double-stranded nucleic acids and synthesize a new strand in a template-based manner. Strand displacing polymerases displace a complementary strand from a template strand and catalyze new strand synthesis. Strand displacing polymerases include mesophilic and thermophilic polymerases. Strand displacing polymerases include wild type enzymes, and variants including exonuclease minus mutants, mutant versions, chimeric enzymes and truncated enzymes. Examples of strand displacing polymerases include phi29 DNA polymerase, large fragment of Bst DNA polymerase, large fragment of Bsu DNA polymerase (exo-), Bca DNA polymerase (exo-), Klenow fragment of E. coli DNA polymerase, T5 polymerase, M-MuLV reverse transcriptase, HIV viral reverse transcriptase, Deep Vent DNA polymerase and KOD DNA polymerase. The phi29 DNA polymerase can be wild type phi29 DNA polymerase (e.g., MagniPhi™ from Expedeon™), or variant EquiPhi29™ DNA polymerase (e.g., from Thermo Fisher Scientific™), or chimeric QualiPhi™ DNA polymerase (e.g., from 4Basebio™).

The terms “nucleic acid”, “polynucleotide” and “oligonucleotide” and other related terms used herein are used interchangeably and refer to polymers of nucleotides and are not limited to any particular length. Nucleic acids include recombinant and chemically synthesized forms. Nucleic acids can be isolated. Nucleic acids include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and chimeric forms containing DNA and RNA. Nucleic acids can be single-stranded or double-stranded. Nucleic acids comprise polymers of nucleotides, where the nucleotides include natural or non-natural bases and/or sugars. Nucleic acids comprise naturally-occurring internucleosidic linkages, for example phosphdiester linkages. Nucleic acids comprise non-natural internucleoside linkages, including phosphorothioate, phosphorothiolate, or peptide nucleic acid (PNA) linkages. In some embodiments, nucleic acids comprise a one type of polynucleotides or a mixture of two or more different types of polynucleotides.

The term “operably linked” and “operably joined” or related terms as used herein refers to juxtaposition of components. The juxtapositioned components can be linked together covalently. For example, two nucleic acid components can be enzymatically ligated together where the linkage that joins together the two components comprises phosphodiester linkage. A first and second nucleic acid component can be linked together, where the first nucleic acid component can confer a function on a second nucleic acid component. For example, linkage between a primer binding sequence and a sequence of interest forms a nucleic acid library molecule having a portion that can bind to a primer. In another example, a transgene (e.g., a nucleic acid encoding a polypeptide or a nucleic acid sequence of interest) can be ligated to a vector where the linkage permits expression or functioning of the transgene sequence contained in the vector. In some embodiments, a transgene is operably linked to a host cell regulatory sequence (e.g., a promoter sequence) that affects expression of the transgene. In some embodiments, the vector comprises at least one host cell regulatory sequence, including a promoter sequence, enhancer, transcription and/or translation initiation sequence, transcription and/or translation termination sequence, polypeptide secretion signal sequences, and the like. In some embodiments, the host cell regulatory sequence controls expression of the level, timing and/or location of the transgene.

The terms “linked”, “joined”, “attached”, “appended” and variants thereof comprise any type of fusion, bond, adherence, or association between any combination of compounds or molecules that is of sufficient stability to withstand use in the particular procedure. The procedure can include, but is not limited to: nucleotide binding; nucleotide incorporation; de-blocking (e.g., removal of chain-terminating moiety); washing; removing; flowing; detecting; imaging and/or identifying. Such linkage can comprise, for example, covalent, ionic, hydrogen, dipole-dipole, hydrophilic, hydrophobic, or affinity bonding, bonds or associations involving van der Waals forces, mechanical bonding, and the like. In some embodiments, such linkage occurs intramolecularly, for example linking together the ends of a single-stranded or double-stranded linear nucleic acid molecule to form a circular molecule. In some embodiments, such linkage can occur between a combination of different molecules, or between a molecule and a non-molecule, including but not limited to: linkage between a nucleic acid molecule and a solid surface; linkage between a protein and a detectable reporter moiety; linkage between a nucleotide and detectable reporter moiety; and the like. Some examples of linkages can be found, for example, in Hermanson, G., “Bioconjugate Techniques”, Second Edition (2008); Aslam, M., Dent, A., “Bioconjugation: Protein Coupling Techniques for the Biomedical Sciences”, London: Macmillan (1998); Aslam, M., Dent, A., “Bioconjugation: Protein Coupling Techniques for the Biomedical Sciences”, London: Macmillan (1998).

The term “primer” and related terms used herein refers to an oligonucleotide that is capable of hybridizing with a DNA and/or RNA polynucleotide template to form a duplex molecule. Primers can be single-stranded along their entire length or have single-stranded and double-stranded portions. Primers comprise natural nucleotides and/or nucleotide analogs. Primers can be recombinant nucleic acid molecules. Primers may have any length, but typically range from 4-50 nucleotides. A typical primer comprises a 5′ end and 3′ end. The 3′ end of the primer can include a 3′ OH moiety which serves as a nucleotide polymerization initiation site in a polymerase-catalyzed primer extension reaction. Alternatively, the 3′ end of the primer can lack a 3′ OH moiety or can include a terminal 3′ blocking group that inhibits nucleotide polymerization in a polymerase-catalyzed reaction. Any one nucleotide, or more than one nucleotide, along the length of the primer can be labeled with a detectable reporter moiety. A primer can be in solution (e.g., a soluble primer) or can be immobilized to a support (e.g., a capture primer).

The term “template nucleic acid”, “template polynucleotide”, “target nucleic acid” “target polynucleotide”, “template strand” and other variations refer to a nucleic acid strand that serves as the basis nucleic acid molecule for any of the amplification and/or sequencing methods describe herein. The template nucleic acid can be single-stranded or double-stranded, or the template nucleic acid can have single-stranded or double-stranded portions. The template nucleic acid can be obtained from a naturally-occurring source, recombinant form, or chemically synthesized to include any type of nucleic acid analog. The template nucleic acid can be linear, concatemeric, circular, or other forms.

The term “adaptor” and related terms refers to oligonucleotides that can be operably linked (appended) to a target polynucleotide, where the adaptor confers a function to the co-joined adaptor-target molecule. Adaptors comprise DNA, RNA, chimeric DNA/RNA, or analogs thereof. Adaptors can include at least one ribonucleoside residue. Adaptors can be single-stranded, double-stranded, or have single-stranded and/or double-stranded portions. Adaptors can be configured to be linear, stem-looped, hairpin, or Y-shaped forms. Adaptors can be any length, including 4-100 nucleotides or longer. Adaptors can have blunt ends, overhang ends, or a combination of both. Overhang ends include 5′ overhang and 3′ overhang ends. The 5′ end of a single-stranded adaptor, or one strand of a double-stranded adaptor, can have a 5′ phosphate group or lack a 5′ phosphate group. Adaptors can include a 5′ tail that does not hybridize to a target polynucleotide (e.g., tailed adaptor), or adaptors can be non-tailed. At least a portion of the adaptors comprise a known and pre-determined sequence. An adaptor can include a sequence that is complementary to at least a portion of a primer, such as an amplification primer, a sequencing primer, or a capture primer (e.g., soluble or immobilized capture primers). Adaptors can include a random sequence or degenerate sequence. Adaptors can include at least one inosine residue. Adaptors can include at least one phosphorothioate, phosphorothiolate and/or phosphoramidate linkage. Adaptors can include at least one barcode sequence which can be used to distinguish polynucleotides (e.g., insert sequences) from different sample sources in a multiplex assay. Adaptors can include at least one unique identification sequence (e.g., a molecular tag) that can be used to uniquely identify a nucleic acid molecule to which the adaptor is appended. In some embodiments, the unique identification sequence comprises 2-12 (e.g., 3, 4, 5, 6, 7, 8, 9, 10, 11, or 12) or more nucleotides having a known sequence. For example, the unique identification sequence comprises a known random sequence where a nucleotide at each position is randomly selected from nucleotides having a base A, G, C, T or U. Adaptors can include at least one restriction enzyme recognition sequence, including any one or any combination of two or more selected from a group consisting of type I, type II, type III, type IV, type Hs or type IIB.

The term “universal sequence” and related terms refers to a sequence in a nucleic acid molecule that is common among two or more polynucleotide molecules. For example, an adaptor having a universal sequence can be operably joined to a plurality of polynucleotides so that the population of co joined molecules carry the same universal adaptor sequence. Examples of universal adaptor sequences include an amplification primer sequence, a sequencing primer sequence, or a capture primer sequence (e.g., soluble or immobilized capture primers).

When used in reference to nucleic acid molecules, the terms “hybridize” or “hybridizing” or “hybridization” or other related terms refers to hydrogen bonding between two different nucleic acids to form a duplex nucleic acid. Hybridization also includes hydrogen bonding between two different regions of a single nucleic acid molecule to form a self-hybridizing molecule having a duplex region. Hybridization can comprise Watson-Crick or Hoogstein binding to form a duplex double-stranded nucleic acid, or a double-stranded region within a nucleic acid molecule. The double-stranded nucleic acid, or the two different regions of a single nucleic acid, may be wholly complementary, or partially complementary. Complementary nucleic acid strands need not hybridize with each other across their entire length. The complementary base pairing can be the standard A-T or C-G base pairing or can be other forms of base-pairing interactions. Duplex nucleic acids can include mismatched base-paired nucleotides.

When used in reference to nucleic acids, the terms “extend”, “extending”, “extension” and other variants, refers to incorporation of one or more nucleotides into a nucleic acid molecule. Nucleotide incorporation comprises polymerization of one or more nucleotides into the terminal 3′ OH end of a nucleic acid strand, resulting in extension of the nucleic acid strand. Nucleotide incorporation can be conducted with natural nucleotides and/or nucleotide analogs. Typically, but not necessarily, nucleotide incorporation occurs in a template-dependent fashion. Any suitable method of extending a nucleic acid molecule may be used, including primer extension catalyzed by a DNA polymerase or RNA polymerase.

The term “nucleotides” and related terms refers to a molecule comprising an aromatic base, a five-carbon sugar (e.g., ribose or deoxyribose), and at least one phosphate group. Canonical or non-canonical nucleotides are consistent with use of the term. In some embodiments, the nucleotide comprises a monophosphate, diphosphate, or triphosphate, or corresponding phosphate analog. The term “nucleoside” refers to a molecule comprising an aromatic base and a sugar. Nucleotides and nucleosides can be non-labeled or labeled with a detectable reporter moiety.

Nucleotides (and nucleosides) typically comprise a hetero cyclic base including substituted or unsubstituted nitrogen-containing parent heteroaromatic ring which are commonly found in nucleic acids, including naturally-occurring, substituted, modified, or engineered variants, or analogs of the same. The base of a nucleotide (or nucleoside) is capable of forming Watson-Crick and/or Hoogstein hydrogen bonds with an appropriate complementary base. Exemplary bases include, but are not limited to, purines and pyrimidines such as: 2-aminopurine, 2,6-diaminopurine, adenine (A), ethenoadenine, N⁶-Δ²-isopentenyladenine (6iA), N⁶-Δ²-isopentenyl-2-methylthioadenine (2ms6iA), N⁶-methyladenine, guanine (G), isoguanine, N²-dimethylguanine (dmG), 7-methylguanine (7mG), 2-thiopyrimidine, 6-thioguanine (6sG), hypoxanthine and O⁶-methylguanine; 7-deaza-purines such as 7-deazaadenine (7-deaza-A) and 7-deazaguanine (7-deaza-G); pyrimidines such as cytosine (C), 5-propynylcytosine, isocytosine, thymine (T), 4-thiothymine (4sT), 5,6-dihydrothymine, O⁴-methylthymine, uracil (U), 4-thiouracil (4sU) and 5,6-dihydrouracil (dihydrouracil; D); indoles such as nitroindole and 4-methylindole; pyrroles such as nitropyrrole; nebularine; inosines; hydroxymethylcytosines; 5-methycytosines; base (Y); as well as methylated, glycosylated, and acylated base moieties; and the like. Additional exemplary bases can be found in Fasman, 1989, in “Practical Handbook of Biochemistry and Molecular Biology”, pp. 385-394, CRC Press, Boca Raton, Fla.

Nucleotides (and nucleosides) typically comprise a sugar moiety, such as carbocyclic moiety (Ferraro and Gotor 2000 Chem. Rev. 100: 4319-48), acyclic moieties (Martinez, et al., 1999 Nucleic Acids Research 27: 1271-1274; Martinez, et al., 1997 Bioorganic & Medicinal Chemistry Letters vol. 7: 3013-3016), and other sugar moieties (Joeng, et al., 1993 J. Med. Chem. 36: 2627-2638; Kim, et al., 1993 J. Med. Chem. 36: 30-7; Eschenmosser 1999 Science 284:2118-2124; and U.S. Pat. No. 5,558,991). The sugar moiety comprises: ribosyl; 2′-deoxyribosyl; 3′-deoxyribosyl; 2′,3′-dideoxyribosyl; 2′,3′-didehydrodideoxyribosyl; 2′-alkoxyribosyl; 2′-azidoribosyl; 2′-aminoribosyl; 2′-fluororibosyl; 2′-mercaptoriboxyl; 2′-alkylthioribosyl; 3′-alkoxyribosyl; 3′-azidoribosyl; 3′-aminoribosyl; 3′-fluororibosyl; 3′-mercaptoriboxyl; 3′-alkylthioribosyl carbocyclic; acyclic or other modified sugars.

In some embodiments, nucleotides comprise a chain of one, two or three phosphorus atoms where the chain is typically attached to the 5′ carbon of the sugar moiety via an ester or phosphoramide linkage. In some embodiments, the nucleotide is an analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methylphosphoroamidite groups.

As used herein, a “nucleotide unit” or ‘nucleotide moiety” refers to nucleotides (e.g., dATP, dTTP, dGTP, dCTP, or dUTP), or analogs thereof, comprising comprises a base, sugar and at least one phosphate group. Nucleotide units can be attached to the multivalent molecules used in the sequencing reactions described herein. In general, all nucleotide units attached to the same multivalent molecule will have the same identity (e.g., all A, all T, all C, or all G), although the skilled artisan will appreciate that there may be situations in which a multivalent molecule comprising nucleotide units of differing identity will be advantageous.

The term “rolling circle amplification” generally refers to an amplification method that employs a circularized nucleic acid template molecule containing a target sequence of interest, an amplification primer binding sequence, and optionally one or more adaptor sequences such as a sequencing primer binding sequence and/or a sample index sequence. The rolling circle amplification reaction can be conducted under isothermal amplification conditions, and includes the circularized nucleic acid template molecule, an amplification primer, a strand-displacing polymerase, and a plurality of nucleotides, to generate a concatemer containing tandem repeat sequences of the circular template molecule and any adaptor sequences present in the original circularized nucleic acid template molecule. The concatemer can self-collapse to form a nucleic acid nanoball. The shape and size of the nanoball can be further compacted by including a pair of inverted repeat sequences in the circular template molecule, or by conducting the rolling circle amplification reaction with one or more compaction oligonucleotides. One of the advantages of using rolling circle amplification to generate clonal amplicons for a sequencing workflow, is that the repeat copies of the target sequence in the nanoball can be simultaneously sequenced to increase signal intensity. In some embodiments, the rolling circle amplification reaction can be conducted in the presence of a plurality of compaction oligonucleotides having at least four consecutive guanines. The rolling circle amplification reaction generates concatemers comprising repeat copies of the universal binding sequence for the compaction oligonucleotide. At least one compaction oligonucleotide can form a guanine tetrad and hybridize to the universal binding sequences for the compaction oligonucleotide, and the resulting concatemer can fold to form an intramolecular G-quadruplex structure. The concatemers can self-collapse to form compact nanoballs. Formation of the guanine tetrads and G-quadruplexes in the nanoballs may increase the stability of the nanoballs to retain their compact size and shape which can withstand repeated flows of reagents for conducting any of the sequencing workflows described herein.

When used in reference to nucleic acids, the terms “amplify”, “amplifying”, “amplification”, and other related terms include producing multiple copies of an original polynucleotide template molecule, where the copies comprise a sequence that is complementary to the template sequence, and/or the copies comprise a sequence that is the same as the template sequence. In some embodiments, the copies comprise a sequence that is substantially identical to a template sequence, and/or is substantially identical to a sequence that is complementary to the template sequence.

The term “reporter moiety”, “reporter moieties” or related terms refers to a compound that generates, or causes to generate, a detectable signal. A reporter moiety is sometimes called a “label”. Any suitable reporter moiety may be used, including luminescent, photoluminescent, electroluminescent, bioluminescent, chemiluminescent, fluorescent, phosphorescent, chromophore, radioisotope, electrochemical, mass spectrometry, Raman, hapten, affinity tag, atom, or an enzyme. A reporter moiety generates a detectable signal resulting from a chemical or physical change (e.g., heat, light, electrical, pH, salt concentration, enzymatic activity, or proximity events). A proximity event includes two reporter moieties approaching each other, or associating with each other, or binding each other. It is well known to one skilled in the art to select reporter moieties so that each absorbs excitation radiation and/or emits fluorescence at a wavelength distinguishable from the other reporter moieties to permit monitoring the presence of different reporter moieties in the same reaction or in different reactions. Two or more different reporter moieties can be selected having spectrally distinct emission profiles or having minimal overlapping spectral emission profiles. Reporter moieties can be linked (e.g., operably linked) to nucleotides, nucleosides, nucleic acids, enzymes (e.g., polymerases or reverse transcriptases), or support (e.g., surfaces).

A reporter moiety (or label) comprises a fluorescent label or a fluorophore. Exemplary fluorescent moieties which may serve as fluorescent labels or fluorophores include, but are not limited to fluorescein and fluorescein derivatives such as carboxyfluorescein, tetrachlorofluorescein, hexachlorofluorescein, carboxynapthofluorescein, fluorescein isothiocyanate, NHS-fluorescein, iodoacetamidofluorescein, fluorescein maleimide, SAMSA-fluorescein, fluorescein thiosemicarbazide, carbohy drazinomethylthioacetyl-amino fluorescein, rhodamine and rhodamine derivatives such as TRITC, TMR, lissamine rhodamine, Texas Red, rhodamine B, rhodamine 6G, rhodamine 10, NHS-rhodamine, TMR-iodoacetamide, lissamine rhodamine B sulfonyl chloride, lissamine rhodamine B sulfonyl hydrazine, Texas Red sulfonyl chloride, Texas Red hydrazide, coumarin and coumarin derivatives such as AMCA, AMCA-NHS, AMCA-sulfo-NHS, AMCA-HPDP, DCIA, AMCE-hydrazide, BODIPY and derivatives such as BODIPY FL C3-SE, BODIPY 530/550 C3, BODIPY 530/550 C3-SE, BODIPY 530/550 C3 hydrazide, BODIPY 493/503 C3 hydrazide, BODIPY FL C3 hydrazide, BODIPY FL IA, BODIPY 530/551 IA, Br-BODIPY 493/503, Cascade Blue and derivatives such as Cascade Blue acetyl azide, Cascade Blue cadaverine, Cascade Blue ethylenediamine, Cascade Blue hydrazide, Lucifer Yellow and derivatives such as Lucifer Yellow iodoacetamide, Lucifer Yellow CH, cyanine and derivatives such as indolium based cyanine dyes, benzo-indolium based cyanine dyes, pyridium based cyanine dyes, thiozolium based cyanine dyes, quinolinium based cyanine dyes, imidazolium based cyanine dyes, Cy 3, Cy5, lanthanide chelates and derivatives such as BCPDA, TBP, TMT, BHHCT, BCOT, Europium chelates, Terbium chelates, Alexa Fluor dyes, DyLight dyes, Atto dyes, LightCycler Red dyes, CAL Flour dyes, JOE and derivatives thereof, Oregon Green dyes, WellRED dyes, IRD dyes, phycoerythrin and phycobilin dyes, Malachite green, stilbene, DEG dyes, NR dyes, near-infrared dyes and others known in the art such as those described in Haugland, Molecular Probes Handbook, (Eugene, Oreg.) 6th Edition; Lakowicz, Principles of Fluorescence Spectroscopy, 2nd Ed., Plenum Press New York (1999), or Hermanson, Bioconjugate Techniques, 2nd Edition, or derivatives thereof, or any combination thereof. Cyanine dyes may exist in either sulfonated or non-sulfonated forms, and consist of two indolenin, benzo-indolium, pyridium, thiozolium, and/or quinolinium groups separated by a polymethine bridge between two nitrogen atoms. Commercially available cyanine fluorophores include, for example, Cy3, (which may comprise 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-1,3-dihydro-2H-indol-2-ylidene}prop-1-en-1-yl)-3,3-dimethyl-3H-indolium or 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-5-sulfo-1,3-dihydro-2H-indol-2-ylidene}prop-1-en-1-yl)-3,3-dimethyl-3H-indolium-5-sulfonate), Cy5 (which may comprise 1-(6-((2,5-dioxopyrrolidin-1-yl)oxy)-6-oxohexyl)-2-((1E,3E)-5-((E)-1-(6-((2,5-dioxopyrrolidin-1-yl)oxy)-6-oxohexyl)-3,3-dimethyl-5-indolin-2-ylidene)penta-1,3-dien-1-yl)-3,3-dimethyl-3H-indol-1-ium or 1-(6-((2,5-dioxopyrrolidin-1-yl)oxy)-6-oxohexyl)-2-((1E,3E)-5-((E)-1-(6-((2,5-dioxopyrrolidin-1-yl)oxy)-6-oxohexyl)-3,3-dimethyl-5-sulfoindolin-2-ylidene)penta-1,3-dien-1-yl)-3,3-dimethyl-3H-indol-1-ium-5-sulfonate), and Cy7 (which may comprise 1-(5-carboxypentyl)-2-[(1E,3E,5E,7Z)-7-(1-ethyl-1,3-dihydro-2H-indol-2-ylidene)hepta-1,3,5-trien-1-yl]-3H-indolium or 1-(5-carboxypentyl)-2-[(1E,3E,5E,7Z)-7-(1-ethyl-5-sulfo-1,3-dihydro-2H-indol-2-yl]dene)hepta-1,3,5-trien-1-yl1-3H-indolium-5-sulfonate), where “Cy” stands for ‘cyanine’, and the first digit identifies the number of carbon atoms between two indolenine groups. Cy2 which is an oxazole derivative rather than indolenin, and the benzo-derivatized Cy3.5, Cy5.5 and Cy7.5 are exceptions to this rule.

In some embodiments, the reporter moiety can be a FRET pair, such that multiple classifications can be performed under a single excitation and imaging step. As used herein, FRET may comprise excitation exchange (Forster) transfers, or electron-exchange (Dexter) transfers.

The term “support” as used herein refers to a substrate that is designed for deposition of biological molecules or biological samples for assays and/or analyses. Examples of biological molecules to be deposited onto a support include nucleic acids (e.g., DNA, RNA, and combinations thereof), polypeptides, saccharides, lipids, a single cell or multiple cells. Examples of biological samples include but are not limited to saliva, phlegm, mucus, blood, plasma, serum, urine, stool, sweat, tears and fluids from tissues or organs.

In some embodiments, the support is solid, semi-solid, or a combination of both. In some embodiments, the support is porous, semi-porous, non-porous, or any combination of porosity. In some embodiments, the support can be substantially planar, concave, convex, or any combination thereof. In some embodiments, the support can be cylindrical, for example comprising a capillary or interior surface of a capillary.

In some embodiments, the surface of the support can be substantially smooth. In some embodiments, the support can be regularly or irregularly textured, including bumps, etched, pores, three-dimensional scaffolds, or any combination thereof.

In some embodiments, the support comprises a bead having any shape, including spherical, hemi-spherical, cylindrical, barrel-shaped, toroidal, disc-shaped, rod-like, conical, triangular, cubical, polygonal, tubular or wire-like.

The support can be fabricated from any material, including but not limited to glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. In some embodiments, the support comprises a polymer, e.g., a synthetic polymer. In some embodiments, the support comprises glass. In some embodiments, the support comprises plastic. Various compositions of both glass and plastic substrates are contemplated.

In some embodiments, the present disclosure provides a plurality (e.g., two or more) of nucleic acid template molecules immobilized to a support. In some embodiments, the immobilized plurality of nucleic acid template molecules has the same sequence. In some embodiments, the immobilized plurality of nucleic acid template molecules has different sequences. In some embodiments, individual nucleic acid template molecules in the plurality of nucleic acid template molecules are immobilized to a different site on the support. In some embodiments, two or more individual nucleic acid template molecules in the plurality of nucleic acid templates are immobilized to a site on the support.

The term “array” refers to a support comprising a plurality of sites located at pre-determined locations on the support to form an array of sites. The sites can be discrete and separated by interstitial regions. In some embodiments, the pre-determined sites on the support can be arranged in one dimension, e.g., in a row or a column. In some embodiments, the pre-determined sites on the support are arranged in two dimensions, e.g., in rows and columns. In some embodiments, the plurality of pre-determined sites is arranged on the support in an organized fashion. In some embodiments, the plurality of pre-determined sites is arranged in any organized pattern, including rectilinear, hexagonal patterns, grid patterns, patterns having reflective symmetry, patterns having rotational symmetry, or the like. The pitch between different pairs of sites can be that same or can vary. In some embodiments, the support comprises at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, where the sites are located at pre-determined locations on the support. In some embodiments, a plurality of pre-determined sites on the support (e.g., 10²-10¹⁵ sites or more) are immobilized with nucleic acid template molecules to form a nucleic acid template array. In some embodiments, the nucleic acid template molecules that are immobilized at a plurality of pre-determined sites by hybridization to immobilized surface capture primers. In some embodiments, the nucleic acid template molecules are covalently attached to the surface capture primers. In some embodiments, the nucleic acid template molecules that are immobilized at a plurality of pre-determined sites, for example, immobilized at 10²-10¹⁵ sites (e.g., 10² sites, 10³ sites, 10⁴ sites, 10⁵ sites, 10⁶ sites, 10⁷ sites, 10⁸ sites, 10⁹ sites, 10¹⁰ sites, 10¹¹ sites, 10¹² sites, 10¹³ sites, 10¹⁴ sites, or 10¹⁵ sites) or more. In some embodiments, the immobilized nucleic acid template molecules are clonally amplified to generate immobilized nucleic acid clusters at the plurality of pre-determined sites. In some embodiments, individual immobilized nucleic acid clusters comprise linear clusters. In some embodiments, individual immobilized nucleic acid clusters comprise single-stranded or double-stranded concatemers.

In some embodiments, a support comprising a plurality of sites located at random locations on the support is referred to herein as a support having randomly located sites thereon. The location of the randomly located sites on the support are not pre-determined. The plurality of randomly located sites is arranged on the support in a disordered and/or unpredictable fashion. In some embodiments, the support comprises at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, where the sites are randomly located on the support. In some embodiments, a plurality of randomly located sites on the support (e.g., 10²-10¹⁵ sites or more (e.g., 10² sites, 10³ sites, 10⁴ sites, 10⁵ sites, 10⁶ sites, 10⁷ sites, 10⁸ sites, 10⁹ sites, 10¹⁰ sites, 10¹¹ sites, 10¹² sites, 10¹³ sites, 10¹⁴ sites, 10¹⁵ sites, or more)) are immobilized with nucleic acid template molecules. In some embodiments, the nucleic acid template molecules are immobilized at a plurality of randomly located sites by hybridization to immobilized surface capture primers. In some embodiments, the nucleic acid template molecules are covalently attached to the surface capture primers. In some embodiments, the nucleic acid templates that are immobilized at a plurality of randomly located sites, for example, immobilized at 10²-10¹⁵ sites or more (e.g., 10² sites, 10³ sites, 10⁴ sites, 10⁵ sites, 10⁶ sites, 10⁷ sites, 10⁸ sites, 10⁹ sites, 10¹⁰ sites, 10¹¹ sites, 10¹² sites, 10¹³ sites, 10¹⁴ sites, 10¹⁵ sites, or more). In some embodiments, the immobilized nucleic acid templates are clonally amplified to generate immobilized nucleic acid clusters at the plurality of randomly located sites. In some embodiments, individual immobilized nucleic acid clusters comprise linear clusters. In some embodiments, individual immobilized nucleic acid clusters comprise single-stranded or double-stranded concatemers.

In some embodiments, the plurality of immobilized surface capture primers on the support (e.g., located at pre-determined or random locations on the support) are in fluid communication with each other to permit flowing a solution of reagents (e.g., nucleic acid template molecules, soluble primers, enzymes, nucleotides, divalent cations, buffers, and the like) onto the support, so that the plurality of immobilized surface capture primers on the support can be essentially simultaneously reacted with the reagents in a massively parallel manner. In some embodiments, the fluid communication of the plurality of immobilized surface capture primers can be used to conduct nucleic acid amplification reactions (e.g., RCA, MDA, PCR, and/or bridge amplification) essentially simultaneously on the plurality of immobilized surface capture primers.

In some embodiments, the plurality of immobilized nucleic acid clusters on the support are in fluid communication with each other to permit flowing a solution of reagents (e.g., enzymes, nucleotides, divalent cations, and the like) onto the support so that the plurality of immobilized nucleic acid clusters on the support can be essentially simultaneously reacted with the reagents in a massively parallel manner. In some embodiments, the fluid communication of the plurality of immobilized nucleic acid clusters can be used to conduct nucleotide binding assays and/or conduct nucleotide polymerization reactions (e.g., primer extension or sequencing) essentially simultaneously on the plurality of immobilized nucleic acid clusters, and optionally to conduct detection and imaging for massively parallel sequencing.

In some embodiments, the term “immobilized” and related terms refer to nucleic acid molecules that are attached to a support through covalent bond or non-covalent interaction, or attached to a coating on the support, or buried within a matrix formed by a coating on the support, where the nucleic acid molecules include surface capture primers, nucleic acid template molecules and extension products of capture primers. Extension products of capture primers include, without limitation, nucleic acid concatemers (e.g., nucleic acid clusters). The nucleic acid molecules can be immobilized at pre-determined or random locations on the support. The nucleic acid molecules can be immobilized at pre-determined or random locations on or within a coating passivated on the support.

In some embodiments, the term “immobilized” and related terms refer to enzymes (e.g., polymerases) that are attached to a support through covalent bond or non-covalent interaction, or attached to a coating on the support, or buried within a matrix formed by a coating on the support. The enzymes can be immobilized at pre-determined or random locations on the support. The enzymes can be immobilized at pre-determined or random locations on or within a coating passivated on the support.

In some embodiments, one or more nucleic acid template molecules are immobilized on the support, for example, immobilized at the sites on the support. In some embodiments, the one or more nucleic acid template molecules are clonally amplified. In some embodiments, the one or more nucleic acid template molecules are clonally amplified off the support (e.g., in-solution). In some embodiments, following clonal amplification, the one or more nucleic acid template molecules are deposited onto the support and immobilized on the support. In some embodiments, the clonal amplification reaction of the one or more nucleic acid template molecules is conducted on the support, resulting in immobilization on the support. In some embodiments, the one or more nucleic acid template molecules are clonally amplified (e.g., in solution or on the support) using a nucleic acid amplification reaction. In some embodiments, the nucleic acid amplification reaction includes any one or any combination of: polymerase chain reaction (PCR), multiple displacement amplification (MDA), transcription-mediated amplification (TMA), nucleic acid sequence-based amplification (NASBA), strand displacement amplification (SDA), real-time SDA, bridge amplification, isothermal bridge amplification, rolling circle amplification (RCA), circle-to-circle amplification, helicase-dependent amplification, recombinase-dependent amplification, and/or single-stranded binding (SSB) protein-dependent amplification. Other suitable methods of nucleic acid amplification are known in the art.

The term “surface primer” and related terms refers to single-stranded oligonucleotides that are immobilized to a support and comprise a sequence that can hybridize to at least a portion of a nucleic acid template molecule. Surface capture primers can be used to immobilize template molecules to a support via hybridization. Surface capture primers can be immobilized to a support in a manner that resists primer removal during flowing, washing, aspirating, and changes in temperature, pH, salts, chemical and/or enzymatic conditions. Typically, but not necessarily, the 5′ end of a surface capture primer can be immobilized to a support or to a coating on the support (or embedded in a coating on the support). Alternatively, an interior portion or the 3′ end of a surface capture primer can be immobilized to a support.

The sequence of surface capture primers can be wholly or partially complementary along their length to at least a portion of the nucleic acid template molecule. In some embodiments, a support can include a plurality of immobilized surface capture primers having the same sequence. In some embodiments, a support can include a plurality of immobilized surface capture primers having two or more different sequences. Surface capture primers can be any length, for example, 4-50 nucleotides, 50-100 nucleotides, 100-150 nucleotides, or longer lengths. The skilled artisan will appreciate suitable surface capture primer lengths dependent upon, e.g., the template molecule, properties of the surface capture primer, etc.

A surface capture primer can have a terminal 3′ nucleotide having a sugar 3′ OH moiety which is extendible for nucleotide polymerization (e.g., polymerase catalyzed polymerization). A surface capture primer can have a terminal 3′ nucleotide having the 3′ sugar position linked to a chain-terminating moiety that inhibits nucleotide polymerization. The 3′ chain-terminating moiety can be removed (e.g., de-blocked) to convert the 3′ end to an extendible 3′ OH end using a de-blocking agent. Examples of chain terminating moieties include, without limitation, alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. Azide type chain terminating moieties include, for example and without limitation, azide, azido and azidomethyl groups. Examples of de-blocking agents include, without limitation, a phosphine compound, such as Tris(2-carboxyethyl)phosphine (TCEP) and bis-sulfo triphenyl phosphine (BS-TPP), for chain-terminating groups azide, azido and azidomethyl groups. Examples of de-blocking agents include, without limitation, tetrakis(triphenylphosphine)palladium(0) (Pd(PPh₃)₄) with piperidine, or with 2,3-Dichloro-5,6-dicyano-1,4-benzo-quinone (DDQ), for chain-terminating groups alkyl, alkenyl, alkynyl and allyl. Examples of a de-blocking agent includes, without limitation, Pd/C for chain-terminating groups aryl and benzyl. Examples of de-blocking agents include, without limitation, phosphine, beta-mercaptoethanol or dithiothritol (DTT), for chain-terminating groups amine, amide, keto, isocyanate, phosphate, thio and disulfide. Examples of de-blocking agents include, without limitation, potassium carbonate (K₂CO₃) in MeOH, triethylamine in pyridine, and Zn in acetic acid (AcOH), for carbonate chain-terminating groups. Examples of de-blocking agents include, without limitation, tetrabutylammonium fluoride, pyridine-HF, with ammonium fluoride, and triethylamine trihydrofluoride, for chain-terminating groups urea and silyl.

The term “sequencing” and related terms refers to a method for obtaining nucleotide sequence information from a nucleic acid molecule, typically by determining the identity of at least some nucleotides (including their nucleobase components) within the nucleic acid molecule. In some embodiments, the sequence information of a given region of a nucleic acid molecule includes identifying each and every nucleotide within a region that is sequenced. In some embodiments, sequencing information determines only some of the nucleotides within a region, while the identity of some nucleotides remains unknown, e.g., undetermined or incorrectly determined. Any suitable method of sequencing known in the art may be used. In an exemplary embodiment, sequencing can include label-free methods. In another embodiment, sequencing can include ion based sequencing methods. In some embodiments, sequencing can include labeled, e.g., dye-containing nucleotide or fluorescent based nucleotide, sequencing methods. In some embodiments, sequencing can include polony-based sequencing or bridge sequencing methods. In some embodiments, the sequencing employs polymerases and multivalent molecules for generating at least one avidity complex, wherein individual multivalent molecules comprise a plurality of nucleotide units tethered to a core. In some embodiments, the sequencing employs polymerases and free nucleotides for performing sequencing-by-synthesis. In some embodiments, the sequencing employs a ligase enzyme and a plurality of sequence-specific oligonucleotides for performing sequence-by-ligation.

In some embodiments, the base calling from sequencing data can be assessed for accuracy and quality. Q-score is a measure of data quality. In some embodiments, the Q-score can be defined as a Phred quality score. In some embodiments, the Q-score is based on a logarithmic scale. In certain embodiments, it is defined as Q=−10 log(P) where P is the error probability. For example, Q10 represents 10% error, Q20 represents 1% error, Q30 represents error and Q40 represent 0.01% error. In another example, Q10 is one error in 10, Q20 is one error in 100, Q30 is one error in 1,000, Q40 is one error in 10,000, and Q50 is one error in 100,000.

### INTRODUCTION

In a pairwise sequencing workflow, low quality base calls for T bases when sequencing the first strand (e.g., R1 reads), and low-quality base calls for A bases when sequencing corresponding positions on the complementary second strand (e.g., R2 reads) have been observed. Many of the low-quality T base calls on the first strand sequence align with C bases in a known reference sequence. Without wishing to be bound by theory, it was hypothesized that some of the bases in the library molecules were deaminated, which lead to the base substitutions including C:G to T:A transitions.

Deamination is generally the removal of an amino group from a molecule. With respect to nucleotide bases, cytosine (C) can be deaminated to generate uracil (U) where uracil can base pair with adenine (A), guanine (G) can be deaminated to generate xanthine where xanthine can base pair with cytosine (C), and adenine (A) can be deaminate to generate hypoxanthine where hypoxanthine can base pair with cytosine (C).

Workflows for preparing nucleic acid library molecules involve numerous steps that include conditions that can cause deamination of nucleotide bases. For example, deamination can be caused by the presence of deaminase enzymes at any stage in the library prep workflow. As another example, any of the library preparation buffers having a low pH can cause base deamination. In another example, high temperatures employed for PCR can cause base deamination. In another example, mechanical shearing forces that are used to fragment input nucleic acids can generate damaging free radicals which lead to deamination. Exemplary mechanical forces include, without limitation, sonication force, acoustic force, nebulizing force, shearing force, and cavitation force. In another example, certain chemicals such as bisulfites can cause deamination. The skilled artisan will recognize that nucleotide base deamination can be generated by many other conditions.

The present disclosure provides compositions and methods for removing deaminated bases in nucleic acid library molecules. The compositions and methods described herein can be applied to any type of nucleic acid library molecules, including, for example, linear or circularized library molecules, and library molecules for sequencing in a massively parallel manner.

Preparing Linear Library Molecules with Reduced Deaminated Nucleotide Bases

In one aspect, the present disclosure provides methods for preparing nucleic acid library molecules having reduced deaminated nucleotide bases. In some embodiments, a linear and/or a circularized nucleic acid library can be prepared, and the library molecules can be treated with a reagent that removes deaminated bases.

In some embodiments, methods for preparing nucleic acid library molecules having reduced deaminated nucleotide bases generally comprises the steps: preparing a plurality of linear nucleic acid library molecules wherein at least one of the linear library molecules comprises at least one deaminated base; contacting the plurality of linear library molecules with a reagent that removes deaminated bases, thereby generating at least one linear library molecule having an abasic site. In some embodiments, the methods further comprise circularizing individual linear library molecules, including the at least one linear library molecule carrying the abasic site, to generate a plurality of circularized nucleic acid library molecules. In some embodiments, the plurality of circularized nucleic acid library molecules may include a circularized molecules carrying at least one abasic site. In some embodiments, the plurality of circularized nucleic acid library molecules may not include a circularized molecule carrying at least one abasic site. In some embodiments, the method further comprises contacting the plurality of circularized library molecules with a reagent that removes deaminated bases to generate at least one circularized library molecule carrying at least one abasic site.

In some embodiments, a nucleic acid library can be prepared by fragmenting input nucleic acids. In some embodiments, input nucleic acids comprise DNA, RNA, or cDNA. In some embodiments, the input nucleic acids comprise nucleic acids having the same sequence or different sequence. In some embodiments, the input nucleic acids comprise single-stranded or double-stranded nucleic acids. In some embodiments, the input nucleic acid can be fragmented using mechanical force, enzymatic (e.g., restriction endonuclease) or chemical fragmentation methods. In some embodiments, the mechanical force comprises sonication force, acoustic force, nebulizing force, shearing force, or cavitation force. In some embodiments, mechanical force can generate free radicals which can deaminate nucleotide bases. In some embodiments, nucleic acid fragments can be generated from RNA using reverse transcriptase to produce RNA hybridized to cDNA. In some embodiments, double-stranded DNA can be prepared by reacting DNA polymerase with the RNA hybridized to cDNA. In some embodiments, DNA fragments can be generated by conducting PCR using template polynucleotides and a pair of PCR primers. In some embodiments, input nucleic acids can be fragmented using an enzyme that generates single-stranded nicks and another enzyme that catalyzes double-stranded cleavage. An exemplary enzyme mixture includes FRAGMENTASE™ (e.g., from New England Biolabs™). In yet another embodiment, the nucleic acid fragments comprise circulating cell-free DNA (e.g., double-stranded cfDNA) which has not been subjected to a fragmentation procedure. The cell-free DNA can be 50-200 bp in length, e.g., about 50 bp, 60 bp, 70 bp, 80 bp, 90 bp, 100 bp, 110 bp, 120 bp, 130 bp, 140 bp, 150 bp, 160 bp, 170 bp, 180 bp, 190 bp, or 200 bp. In some embodiments, the nucleic acid fragments can be size-selected. In some embodiments, the nucleic acid fragments lack size selection. A skilled artisan will recognize that the nucleic acid fragments can be generated using any of these methods. In some embodiments, the fragments can be 50-1000 bp in length or larger than 1000 bp, e.g., about 50 bp, 75 bp, 100 bp, 150 bp, 200 bp, 250 bp, 300 bp, 400 bp, 500 bp, 600 bp, 700 bp, 800 bp, 900 bp, 1000 bp, or more than 1000 bp. The nucleic acid fragments can include a heterogeneous mixture of fragments having 5′ overhang ends, 3′ overhang ends, and/or blunt ends.

In some embodiments, a nucleic acid library can be prepared by modifying (e.g., end repairing) the terminal 5′ and/or 3′ ends of the fragmented nucleic acids to convert overhang ends to blunt ends and/or to generate desirable overhang ends. For example, the nucleic acid fragments can be treated with at least one enzyme to remove 3′ overhang ends. Suitable enzymes for use include, for example and without limitation, DNA polymerase I, Large (Klenow) fragment, T4 DNA polymerase and/or mung bean nuclease. In some embodiments, the nucleic acid fragments can be treated with at least one enzyme to fill-in 5′ overhang ends. Suitable enzymes for use include, for example and without limitation, T4 DNA polymerase, Tfi DNA polymerase, Tli DNA polymerase, Taq DNA polymerase, Large (Klenow) fragment, phi29 DNA polymerase and/or Mako DNA polymerase. Any of these polymerases can be heat-stable or heat-labile enzymes. In some embodiments, the nucleic acid fragments can be treated with at least one enzyme to remove 5′ overhang ends, e.g., using SI nuclease. In some embodiments, the nucleic acid fragments can be treated with at least one enzyme to remove 5′ or 3′ overhang ends, e.g., using mung bean nuclease.

In some embodiments, a nucleic acid library can be prepared by phosphorylating the 5′ ends of the fragmented nucleic acids, or to remove 5′ or 3′ phosphates. For example, the nucleic acid fragments can be treated with T4 polynucleotide kinase to phosphorylate the 5′ end of at least one strand of duplex DNA. In some embodiments, the nucleic acid fragments can be treated with a phosphatase to remove a 5′ or 3′ phosphate using, for example and without limitation, shrimp alkaline phosphatase, calf intestinal alkaline phosphatase, bacterial alkaline phosphatase, Antarctic phosphatase, and/or placental alkaline phosphatase.

In some embodiments, a nucleic acid library can be prepared by adding a non-template tail. In some embodiments, an A-tail (e.g., a poly-A tail) comprising one or more non-template adenosine nucleotides can be appended to the end of the linear library molecules using a DNA polymerase, such as for example and without limitation, a Taq DNA polymerase (or a derivative thereof), a Tfi (exo-minus) DNA polymerase, a large fragment Klenow (e.g., 3′ to 5′ exo-minus), or a T4 DNA polymerase.

In some embodiments, a single non-template A-tail (e.g., a poly-A tail) can be appended to the 3′ ends of the linear library molecules using proofreading DNA polymerase, Tfi (exo-) DNA polymerase or Pfu DNA polymerase, both in the presence of dATP.

In some embodiments, a nucleic acid library can be prepared by appending at least one adaptor to one end of nucleic acid fragments. In some embodiments, an adaptor comprises an oligonucleotide that can be operably linked (appended) to a nucleic acid fragment, where the adaptor confers a function to the co-joined adaptor-fragment molecule. Adaptors comprise DNA, RNA, chimeric DNA/RNA, or analogs thereof. Adaptors can include at least one ribonucleoside residue. Adaptors can be single-stranded, double-stranded, or have single-stranded and/or double-stranded portions. Adaptors can be configured to be linear, stem-looped, hairpin, or Y-shaped forms. Adaptors can be any length, including 4-100 nucleotides or longer, e.g., about 5, 6, 7, 8, 9, 10, 15, 20, 30, 40, 50, 60, 70, 80, 90, 100, or more nucleotides. Adaptors can have blunt ends, overhang ends, or a combination of both. Overhang ends include 5′ overhang and 3′ overhang ends. In some embodiments, the 5′ end of a single-stranded adaptor, or one strand of a double-stranded adaptor, can have a 5′ phosphate group or lack a 5′ phosphate group. In some embodiments, adaptors can include a 5′ tail that does not hybridize to a nucleic acid fragment (e.g., tailed adaptor), or adaptors can be non-tailed. At least a portion of the adaptors comprise a known and pre-determined sequence. An adaptor can include, for example, a sequence that is complementary to at least a portion of a primer, such as an amplification primer, a sequencing primer, or a capture primer (e.g., soluble or immobilized capture primers). Adaptors can include, for example, a random sequence or degenerate sequence. In some embodiments, adaptors can include at least one inosine residue. In some embodiments, adaptors can include at least one phosphorothioate, phosphorothiolate and/or phosphoramidate linkage. In some embodiments, adaptors can include at least one barcode/index sequence which can be used to distinguish polynucleotides (e.g., insert sequences) from different sample sources in a multiplex assay. In some embodiments, adaptors can include at least one unique identification sequence (e.g., a molecular tag) that can be used to uniquely identify a nucleic acid molecule to which the adaptor is appended. In some embodiments, the unique identification sequence comprises 2-12 (e.g., 3, 4, 5, 6, 7, 8, 9, 10, 11, or 12) or more nucleotides having a known sequence. For example, the unique identification sequence comprises a known random sequence where a nucleotide at each position is randomly selected from nucleotides having a base A, G, C, T or U. Adaptors can include, for example, at least one restriction enzyme recognition sequence. In some embodiments, adaptors can include any one or any combination of two or more restriction enzyme recognition sequences. In some embodiments, the restriction enzyme recognition sequences are selected from a group consisting of type I, type II, type III, type IV, type Hs or type IIB.

In some embodiments, the adaptor can be universal, e.g., having a universal sequence. In some embodiments, a universal sequence and related terms refers to a sequence in a nucleic acid molecule (e.g., an adaptor) that is common among two or more polynucleotide molecules. For example, an adaptor having a universal sequence can be operably joined to a plurality of polynucleotides so that the population of co-joined molecules carry the same universal adaptor sequence. Examples of universal adaptor sequences include, without limitation, an amplification primer sequence, a sequencing primer sequence, or a capture primer sequence (e.g., soluble or immobilized capture primers).

In some embodiments, the adaptors comprise double-stranded nucleic acid Y-shaped adaptors. In certain embodiments, individual double-stranded adaptors comprise a first and second oligonucleotide strand hybridized together, wherein both the first and second oligonucleotide strands comprise a complementary region and a mismatched region, thereby forming a Y-shaped adaptor having a double-stranded annealed portion and a mismatched portion having two single strands.

In some embodiments, the 5′ end of the first and/or second oligonucleotide strands that form the Y-shaped adaptor can be phosphorylated. In some embodiments, the first oligonucleotide strand comprises a first universal adaptor sequence comprising a binding sequence for a first sequencing primer or a complementary sequence thereof. In some embodiments, the second oligonucleotide strand comprises a second universal adaptor sequence comprising a binding sequence for a second sequencing primer or a complementary sequence thereof. In some embodiments, in a population of Y-shaped adaptors, each of the first oligonucleotide stands that form the Y-shaped adaptors have the same sequence. In some embodiments, in a population of Y-shaped adaptors, each of the second oligonucleotide stands that form the Y-shaped adaptors have the same sequence. In some embodiments, the double-stranded annealed region of the Y-shaped adaptor includes at least 4 consecutive based-paired nucleotides. In some embodiments, the double-stranded annealed region includes a terminal end that can be joined to a nucleic acid fragment having a sequence-of-interest via an enzymatic ligation reaction. In some embodiments, the double-stranded annealed region includes a terminal end that is blunt-ended, or the terminal end can have a 5′ or 3′ overhang region. In some embodiments, the first and second oligonucleotide strands of the mismatched portion can be the same length. In some embodiments, the first and second oligonucleotide strands of the mismatched portion can be different lengths.

In some embodiments, the first strand of the double-stranded annealed region of the Y-shaped adaptors comprise at least a portion of a binding sequence for the first sequencing primer (e.g., a reverse or forward sequencing primer) or a complementary sequence thereof. In some embodiments, the second strand of the double-stranded annealed region of the Y-shaped adaptors comprise at least a portion of a binding sequence for the second sequencing primer (e.g., a forward or reverse sequencing primer) or a complementary sequence thereof.

In some embodiments, the first strand of the mismatched region includes at least a portion of a binding sequence for the first sequencing primer (e.g., reverse or forward sequencing primer) or a complementary sequence thereof. In some embodiments, the first strand of the mismatched region further comprises at least a portion of a binding sequence for the first surface primer or a complementary sequence thereof. In some embodiments, the first strand of the mismatched region comprises a universal adaptor sequence comprising a first sample index sequence.

In some embodiments, the second strand of the mismatched region includes at least a portion of a binding sequence for the second sequencing primer (e.g., forward or reverse sequencing primer) or a complementary sequence thereof. In some embodiments, the second strand of the mismatched region further comprises at least a portion of a binding sequence for a second surface primer or a complementary sequence thereof. In some embodiments, the second strand of the mismatched region further comprises a universal adaptor sequence comprising a second sample index sequence.

In some embodiments, only the first strand of the mismatched region comprises a universal adaptor, e.g., which comprises the first sample index sequence. In some embodiments, only the second strand of the mismatched region comprises a universal adaptor, e.g., which comprises the second sample index sequence. In some embodiments, both the first strand of the mismatched region comprises the first sample index sequence, and the second strand of the mismatched region comprises the second sample index sequence.

In some embodiments, double stranded nucleic acid fragments having blunt ends or a 5′ overhang end or a 3′ overhang end can be appended at one end or at both ends, with at least one adaptor using a ligation reaction. The adaptor ligation reaction can include linear double stranded adaptors and/or double-stranded Y-shaped adaptors. In some embodiments, the ligation reaction can be conducted using a T4 DNA ligase, a T3 DNA ligase, or a T7 DNA ligase.

In some embodiments, single-stranded tailed primers can be used to append adaptor sequences to one or both ends of nucleic acid fragments. In some embodiments, the adaptor sequences are appended using at least one primer extension reaction. In some embodiments, the adaptor sequences are appended using PCR. In some embodiments, the 5′ end of the tailed primers comprises the adaptor sequence to be appended to the nucleic acid fragments. In some embodiments, the 5′ end of the tailed primers does not hybridize to the nucleic acid fragments. In some embodiments, the 3′ end of the tailed primers comprises a sequence capable of hybridizing to at least a portion of the nucleic acid fragments. In some embodiments, adaptor sequences can be appended to nucleic acid fragments by conducting at least one primer extension reaction using the single-stranded tailed primers, a polymerase, and a plurality of nucleotides. In some embodiments, the primer extension reaction can employ one type of tailed primers to append adaptor sequences, e.g., to one end of the nucleic acid fragments. In some embodiments, the primer extension reaction can employ two types of tailed primers, e.g., to append adaptor sequences to both ends of the nucleic acid fragments. In some embodiments, the multiple primer extension reactions can be employed using PCR. In certain embodiments, the heat from the PCR reaction can generate nucleic acid library molecules comprising at least one deaminated nucleotide base.

In some embodiments, nucleic acid linear library molecules can be generated using any of the methods described above. In some embodiments, individual library molecules comprise a sequence of interest operably linked on both sides by at least one nucleic acid adaptor sequence. In some embodiments, at least one library molecule carries at least one deaminated nucleotide base. In some embodiments, the library molecules can be treated with a reagent that removes deaminated bases. In some embodiments, the reagent that removes deaminated bases comprises a compound that generates abasic sites at uracil bases in a nucleic acid molecule. For example, and without limitation, DNA glycosylase (UDG) can generate abasic sites at uracil bases. In some embodiments, the reagent that removes deaminated bases comprises a compound that generates a gap at an abasic site in a nucleic acid strand. For example, and without limitation, the gaps can be generated with an enzyme or a mixture of enzymes having lyase activity, e.g., that breaks the phosphodiester backbone at the 5′ and 3′ sides of the abasic site to release the base-free deoxyribose and generate a gap. In certain embodiments, the abasic sites can be removed using AP lyase, Endo IV endonuclease, FPG glycosylase/AP lyase, Endo VIII glycosylase/AP lyase, or a combination thereof. In some embodiments, generating the abasic sites and removal of the abasic sites to generate gaps can be achieved using a mixture of uracil DNA glycosylase and DNA glycosylase-lyase endonuclease VIII. For example, and without limitation, suitable DNA glycosylase-lyase endonuclease VIII may be USER™ (Uracil-Specific Excision Reagent Enzyme from New England Biolabs™) or thermolabile USER™ (also from New England Biolabs™)

Preparing Circularized Library Molecules with Reduced Deaminated Nucleotide Bases

In another aspect, the present disclosure provides methods for preparing nucleic acid library molecules having reduced deaminated nucleotide bases. In some embodiments, linear library molecules can be circularized to generate a circularized nucleic acid library. In some embodiments, the circularized library molecules can be treated with a reagent that removes deaminated bases.

In some embodiments, linear library molecules can be generated using any of the adaptors and any of the adaptor-appending methods described herein. In some embodiments, the linear library molecules can be circularized using intra-molecular ligation methods, padlock probes, or a telomerase. Other suitable methods of circularizing linear molecules are known in the art.

In some embodiments, the ends of single-stranded library molecules can undergo intramolecular ligation using a single-stranded ligase (e.g., CircLigase from Epicentre™ or Lucigen™) to generate a covalently closed circular library molecule. The covalently closed circular library molecule can be subjected to a rolling circle amplification reaction.

In some embodiments, single-stranded library molecules can be circularized using padlock probes. In some embodiments, a padlock probe typically comprises a single-stranded oligonucleotide having a 5′ portion, internal linker portion, and 3′ portion. In the padlock probe, the 5′ and 3′ portions are separately complementary to a target sequence in the linear library molecules, while the linker portion is designed to have little or no complementarity to the target sequence. The 5′ and 3′ portions hybridize to the target sequence, causing the padlock probe to form a circularized molecule. Ligation of the padlock probe, when it is hybridized to the target sequence, forms a covalently closed single-stranded circular nucleic acid molecule. In some embodiments, the internal linker portion can be engineered to include one or more universal adaptor sequences, barcode adaptors, or unique identifier adaptors. In some embodiments, the covalently closed circular library molecule can be subjected to a rolling circle amplification reaction.

In some embodiments, circular DNA molecules can be generated without a nucleic acid ligase. For example, and without limitation, a protelomerase enzyme identifies a target enzyme recognition sequence within a nucleic acid molecule (e.g., a linear library molecule), cleaves the enzyme recognition sequence to generate an end having a 5′ and 3′ exposed cleavage ends, and rejoins 5′ and 3′ cleavage ends of a single exposed end at the target site to form a single linear molecule from the cleaved 5′ and 3′ ends. When this reaction is performed on both ends of a double-stranded nucleic acid molecule (e.g., double-stranded library molecule) having a target enzyme recognition sequence appended at each end, the result is a circularized nucleic acid molecule. In some embodiments, an adaptor carrying the enzyme recognition sequence can be appended to the double-stranded library molecules via ligation or PCR using tailed PCR primers. A number of enzymes or enzyme combinations are compatible with this reaction, including, for example and without limitation, a protelomerase. One suitable type of protelomerase is TelN protelomerase, such as that from E. coli phage Nl. In some embodiments, the covalently closed circular library molecule can be subjected to a rolling circle amplification reaction.

In some embodiments, linear library molecules can be generated using any of the adaptors and any of the adaptor-appending methods described herein. In some embodiments, the linear library molecules can be circularized using a single-stranded splint strand or double-stranded splint adaptor. For example, linear library molecules can be hybridized to single stranded splints which can bring the ends of the linear molecules juxtapositioned to each other for ligation. In another example, linear library molecules can be hybridized to double-stranded splint adaptors each having short and long splint strands. The long splint strands hold the linear library molecules in a circularized form, and the ends of the short splint strands are juxtapositions to the ends of the linear library molecules for ligation.

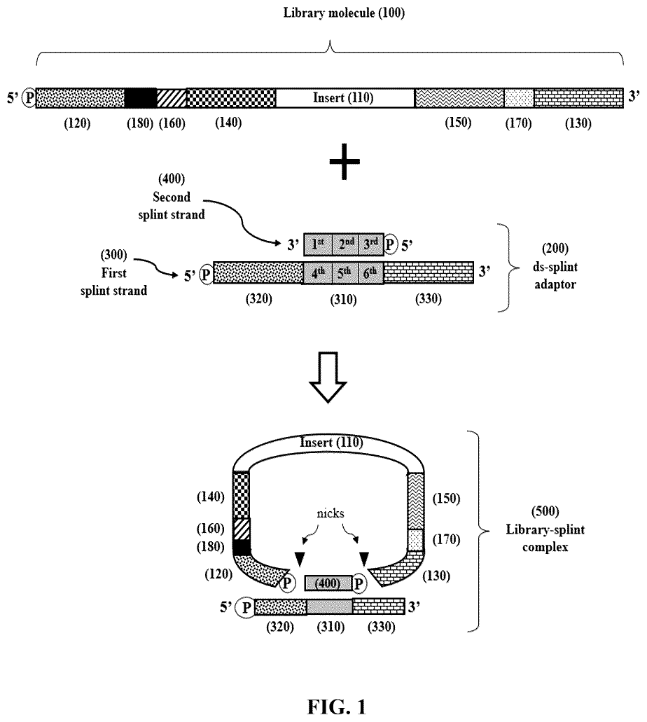

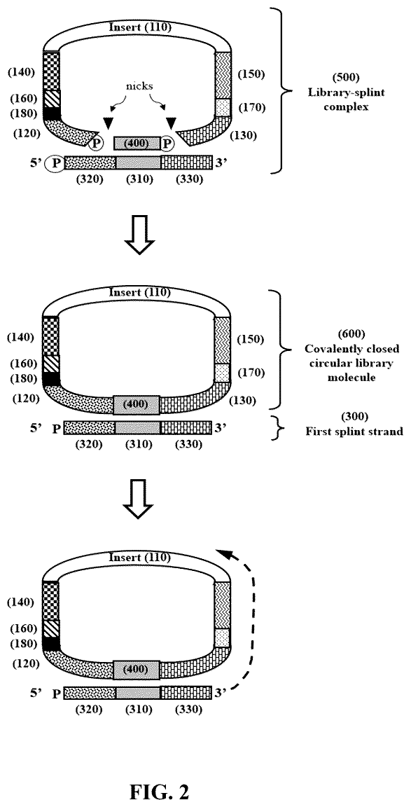

For example, FIG. 1 shows a linear, single-stranded library molecule (100) hybridizing with a double-stranded (ds) splint molecule adaptor (200) thereby circularizing the library molecule to form a library-splint complex (500) with two nicks. The library molecule (100) comprises: a first left universal adaptor sequence (120); a first left unique identification sequence (180); a first left index sequence (160); a second left universal adaptor sequence (140); a sequence of interest insert (110); a second right universal adaptor sequence (150); a first right index sequence (170); and a first right universal adaptor sequence (130). The double-stranded splint molecule comprises a first splint strand (long strand (300)) hybridized to a second splint strand (short strand (400)). The first splint strand comprises a first region (320) that hybridizes with a sequence on one end of the linear single stranded library molecule, and a second region (330) that hybridizes with a sequence on the other end of the linear single stranded library molecule. The internal region (310) of the first splint strand hybridizes to the second splint strand (400). The second splint strand (400) includes three sub-regions, where the first sub-region comprises a universal binding sequence for a third surface primer, the second sub-region comprises a universal binding sequence for a fourth surface primer, and the third sub-region comprises a sample index sequence having 5-20 bases and/or a unique identification sequence having 2-10 or more bases (e.g., NN). The internal region (310) of the first splint strand (300) comprises three sub-regions, where the fourth sub-region hybridizes to the first sub-region of the second splint strand (400), and the fifth sub-region hybridizes to the second sub-region of the second splint strand (400), and the sixth sub-region hybridizes to the third sub-region of the second splint strand (400).

In some embodiments, linear library molecules can be circularized using double-stranded splint adaptors (200) which comprise a first splint strand (long splint strand (300)) and a second splint strand (short splint strand (400)), where the first and second splint strands are hybridized together to form the double-stranded splint adaptor (200) having a double-stranded region and two flanking single-stranded regions (e.g., see FIGS. 1-2). The second splint strand (400) carries the new adaptor sequence(s) to be introduced, such as for example a new universal binding sequence and/or a new index sequence. The first splint strand comprises a first region (320), an internal region (310), and a second region (330). The internal region of the first splint strand (310) is hybridized to the second splint strand (400). The two-flanking single-stranded regions of the double-stranded splinted adaptor (e.g., (320) and (330)) are designed to hybridize to universal adaptor sequences at the ends of a single-stranded linear library molecule (100) having a sequence of interest (110). For example, the first region of the first splint strand (320) is hybridized to one end of the library molecule, and the second region of the first splint strand (330) is hybridized to the other end of the library molecule, thereby circularizing the library molecule to generate a library-splint complex (500) which includes two nicks (e.g., see FIGS. 1-2). The nicks can be enzymatically ligated to generate a covalently closed circular molecule (600) in which the second splint strand (400) is covalently joined at both ends to the library molecule, thereby introducing the new adaptor sequences into the library molecule.

In some embodiments, covalently closed circular library molecules can be generated using any of the methods described above. In some embodiments, individual covalently closed circular library molecules comprise a sequence of interest operably linked on both sides to at least one nucleic acid adaptor sequence. In some embodiments, at least one covalently closed circular library molecule carries at least one deaminated nucleotide base. In some embodiments, the covalently closed circular library molecules can be treated with a reagent that removes deaminated bases. In some embodiments, the reagent that removes deaminated bases comprises a compound that generates abasic sites at uracil bases in a nucleic acid molecule. For example, and without limitation, DNA glycosylase (UDG) can generate abasic sites at uracil bases. In some embodiments, the reagent that removes deaminated bases comprises a compound that generates a gap at abasic sites in a nucleic acid strand. For example, and without limitation, the gaps can be generated with an enzyme or a mixture of enzymes having lyase activity that breaks the phosphodiester backbone at the 5′ and 3′ sides of the abasic site, e.g., to release the base-free deoxyribose and generate a gap. The abasic sites can be removed, for example and without limitation, using AP lyase, Endo IV endonuclease, FPG glycosylase/AP lyase, Endo VIII glycosylase/AP lyase, or a combination thereof. In some embodiments, generating the abasic sites and removal of the abasic sites to generate gaps can be achieved using a mixture of uracil DNA glycosylase and DNA glycosylase-lyase endonuclease VIII. For example, and without limitation, a suitable DNA glycosylase-lyase endonuclease VIII may be USER™ (Uracil-Specific Excision Reagent Enzyme from New England Biolabs™) or thermolabile USER™ (also from New England Biolabs™)

### Methods for Forming a Plurality of Library-Splint Complexes Using Double-Stranded Splint Adaptors

In some embodiments, any of the methods described above can be used to generate nucleic acid linear library molecules each comprising sequences arranged in a 5′ to 3′ order: (i) a first left universal adaptor sequence (120) having a binding sequence for a first surface primer; (ii) a first left index sequence (160); (iii) a second left universal adaptor sequence (140) having a binding sequence for a first sequencing primer; (iv) a sequence of interest (110); (v) a second right universal adaptor sequence (150) having a binding sequence for a second sequencing primer; (vi) a first right index sequence (170); and (vii) a first right universal adaptor sequence (130) having a binding sequence for a second surface primer (e.g., see FIG. 1).

In some embodiments, at least one of the linear library molecules carries at least one deaminated nucleotide base. In some embodiments, prior to circularization, the linear library molecules can be treated with a reagent that removes deaminated bases. In some embodiments, the reagent that removes deaminated bases comprises a compound that generates abasic sites at uracil bases in a nucleic acid molecule. For example, DNA glycosylase (UDG) can generate abasic sites at uracil bases. In some embodiments, the reagent that removes deaminated bases comprises a compound that generates a gap at abasic sites in a nucleic acid strand. For example, and without limitation, the gaps can be generated with an enzyme or a mixture of enzymes having lyase activity that breaks the phosphodiester backbone at the 5′ and 3′ sides of the abasic site to release the base-free deoxyribose and generate a gap. The abasic sites can be removed using, for example and without limitation, AP lyase, Endo IV endonuclease, FPG glycosylase/AP lyase, Endo VIII glycosylase/AP lyase, or a combination thereof. In some embodiments, generating the abasic sites and removal of the abasic sites to generate gaps can be achieved using a mixture of uracil DNA glycosylase and DNA glycosylase-lyase endonuclease VIII. For example, and without limitation, suitable DNA glycosylase-lyase endonuclease VIII may be USER™ (Uracil-Specific Excision Reagent Enzyme from New England Biolabs™) or thermolabile USER™ (also from New England Biolabs™).

In some embodiments, the present disclosure provides methods for forming a plurality of library-splint complexes (500) comprising: (a) providing a plurality of double-stranded splint adaptors (200) wherein individual double-stranded splint adaptors (200) in the plurality comprise a first splint strand (300) hybridized to a second splint strand (400), wherein the double-stranded splint adaptor includes a double-stranded region and two flanking single-stranded regions, wherein the first splint strand comprises a first region (320), an internal region (310), and a second region (330), and wherein the internal region of the first splint strand (310) is hybridized to the second splint strand (400). Exemplary double-stranded splint adaptors (200) are shown in FIGS. 1-2.

In some embodiments, the methods for forming a plurality of library-splint complexes (500) further comprise step (b): hybridizing the plurality of double-stranded splint adaptors with a plurality of single-stranded nucleic acid library molecule (100) wherein individual library molecules include a sequence of interest (110) flanked on one side by at least a first left universal adaptor sequence (120) and flanked on the other side by at least a first right universal adaptor sequence (130) (e.g., FIGS. 1-2). The hybridizing is conducted under a condition suitable for hybridizing the first region of the first splint strand (320) to the at least first left universal adaptor sequence (120) of the library molecule. The hybridizing condition is suitable for hybridizing the second region of the first splint strand (330) to the at least first right universal sequence (130) of the library molecule, thereby circularizing the plurality of library molecules to form a plurality of library-splint complexes (500).

In some embodiments, in the methods for forming a plurality of library-splint complexes (500), the first region of the first splint strand (320) comprises a first universal adaptor sequence which can hybridize to a first universal binding sequence at one end of a linear nucleic acid library molecule. In some embodiments, the first region of the first splint strand (320) includes a first universal adaptor sequence which comprises a universal binding sequence for a forward or reverse sequencing primer, a universal binding sequence for a first or second surface primer, a universal binding sequence for a forward or reverse amplification primer, or a universal binding sequence for a compaction oligonucleotide. In some embodiments, the 5′ end of the first splint strand (300) is phosphorylated. In some embodiments, the 5′ end of the first splint strand (300) lacks a phosphate group. In some embodiments, the 3′ end of the first splint strand (300) includes a terminal 3′ OH group or a terminal 3′ blocking group.

In some embodiments, in the methods for forming a plurality of library-splint complexes (500), the second region of the first splint strand (330) comprises a second universal adaptor sequence which can hybridize to a second universal binding sequence at the other end of the linear nucleic acid library molecule. In some embodiments, the second region of the first splint strand (330) includes a second universal adaptor sequence which comprises a universal binding sequence for a forward or reverse sequencing primer, a universal binding sequence for a first or second surface primer, a universal binding sequence for a forward or reverse amplification primer, or a universal binding sequence for a compaction oligonucleotide. In some embodiments, the 5′ end of the second splint strand (400) is phosphorylated. In some embodiments, the 5′ end of the second splint strand (400) lacks a phosphate group. In some embodiments, the 3′ end of the second splint strand (400) includes a terminal 3′ OH group or a terminal 3′ blocking group.

In some embodiments, in the methods for forming a plurality of library-splint complexes (500), the first region of the first splint strand (320) is hybridized to the at least first left universal adaptor sequence (120) of the library molecule, and a second region of the first splint strand (330) is hybridized to the at least first right universal sequence (130) of the library molecule, thereby circularizing the library molecule to generate a library-splint complex (500). The library-splint complex (500) comprises a first nick between the 5′ end of the library molecule and the 3′ end of the second splint strand (e.g., FIGS. 1-2). The library-splint complex (500) also comprises a second nick between the 5′ end of the second splint strand and the 3′ end of the library molecule (e.g., FIGS. 1-2). In some embodiments, the first and second nicks are enzymatically ligatable.

In some embodiment, in the methods for forming a plurality of library-splint complexes (500), the first region of the first splint strand (320) can hybridize to a sense or anti-sense strand of a double-stranded nucleic acid library molecule. In the library-splint complex (500), the second region of the first splint strand (330) can hybridize to a sense or anti-sense strand of a double-stranded nucleic acid library molecule. In some embodiments, the double-stranded nucleic acid library molecule can be denatured to generate the single-stranded sense and anti-sense library strands.

In some embodiments, in the methods for forming a plurality of library-splint complexes (500), the second splint strand (400) does not hybridize to the sequence of interest (110), and the internal region of the first splint strand (310) does not hybridize to the sequence of interest (110).

In some embodiments, in the methods for forming a plurality of library-splint complexes (500), the first region of the first splint strand (320) does not hybridize to the sequence of interest (110), and the second region of the first splint strand (330) does not hybridize to the sequence of interest (110).

In some embodiments, in the methods for forming a plurality of library-splint complexes (500), the 5′ end of the single-stranded library molecule (100) is phosphorylated or lacks a phosphate group. In some embodiments, the 3′ end of the single-stranded library molecule includes a terminal 3′ OH group or a terminal 3′ blocking group.

In some embodiments, in the methods for forming a plurality of library-splint complexes (500), the nucleic acid library molecule (100) further comprises a second left universal adaptor sequence (140). In some embodiments, the nucleic acid library molecule (100) further comprises a second right universal adaptor sequence (150). In some embodiments, the nucleic acid library molecule (100) can further comprise additional left and/or right universal adaptor sequences.

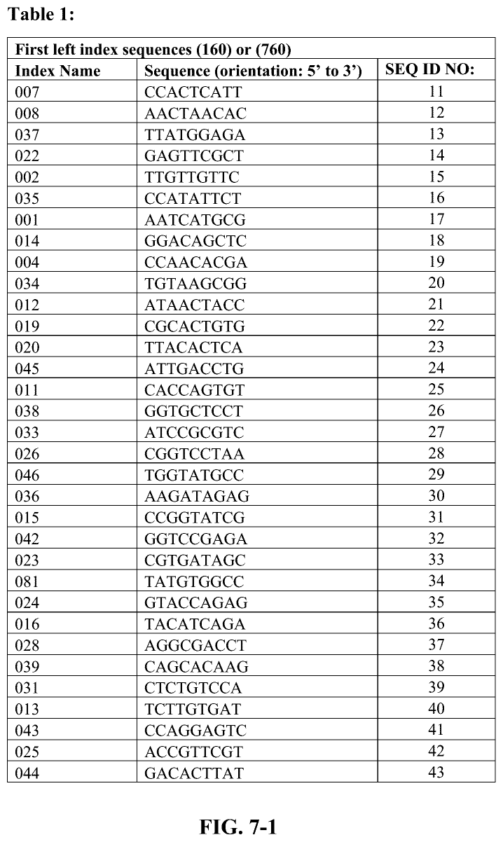

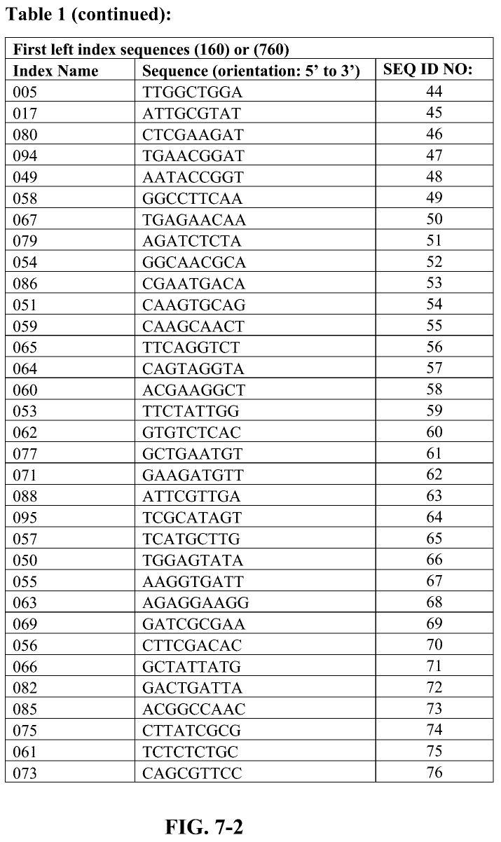

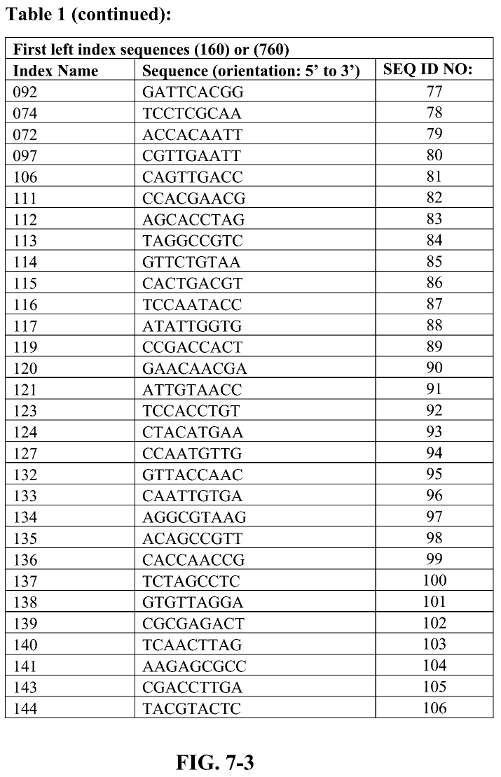

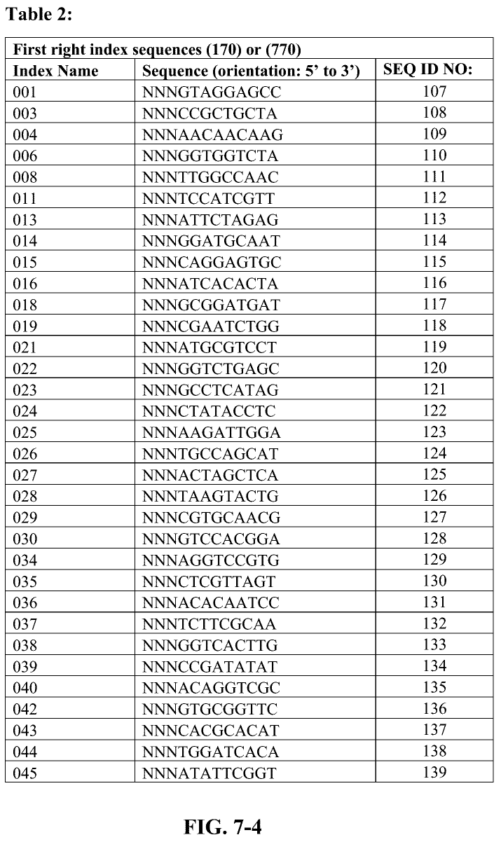

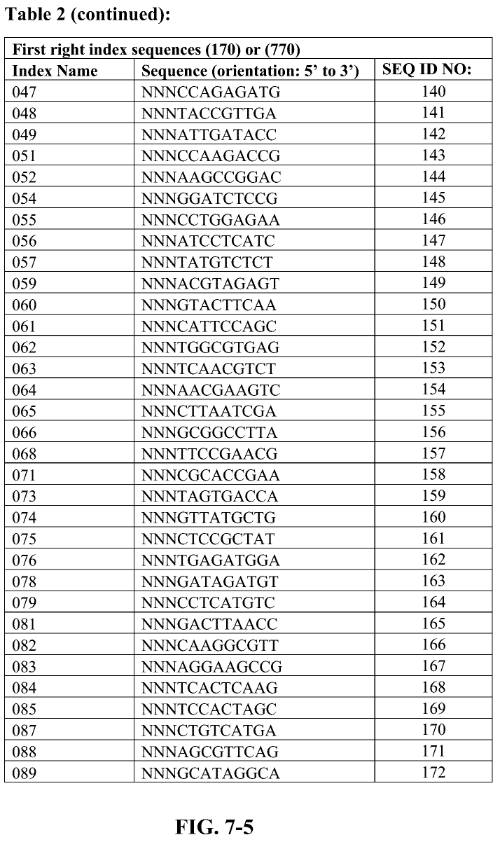

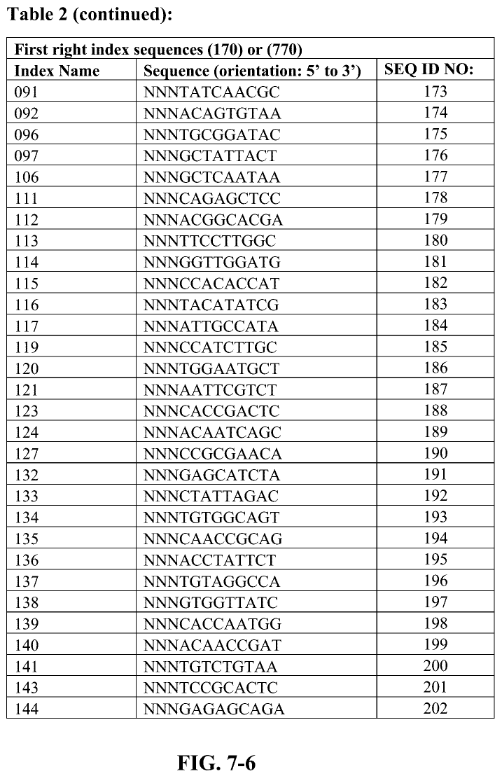

In some embodiments, in the methods for forming a plurality of library-splint complexes (500), the nucleic acid library molecule (100) further comprises a first left index sequence (160). In some embodiments, the nucleic acid library molecule (100) further comprises a first right index sequence (170). In some embodiments, the first left index sequence (160) comprises a sample index sequence. In some embodiments, the first right index sequence (170) comprises another sample index sequence. The sample index sequences can be used to distinguish sequences of interest obtained from different sample sources in a multiplex assay. A list of exemplary first left index sequences (160) and first right index sequences (170) is provided in Tables 1-2 at FIG. 7. In some embodiments, the first left index sequence (160) can include a random sequence (e.g., NNN) or lack a random sequence. In some embodiments, the first right index sequence (170) can include a random sequence (e.g., NNN). Alternatively, in some embodiments, the first right index sequences (170) lack a random sequence.

In some aspects, multiplex workflows are enabled by preparing sample-indexed libraries using one or both index sequences (e.g., left and/or right index sequences). In some embodiments, the first left index sequences (160) and/or first right index sequences (170) can be employed to prepare separate sample-indexed libraries using input nucleic acids isolated from different sources. In some embodiments, the sample-indexed libraries can be pooled together to generate a multiplex library mixture, and the pooled libraries can be amplified and/or sequenced. In some embodiments, the sequences of the insert region along with the first left index sequence (160) and/or first right index sequence (170) can be used to identify the source of the input nucleic acids. In some embodiments, any number of sample-indexed libraries can be pooled together, for example 2-10, 10-50, 50-100, 100-200, or more than 200 (e.g., about 2, 3, 4, 5, 6, 7, 8, 9, 10, 15, 20, 25, 30, 35, 40, 45, 50, 60, 70, 80, 90, 100, 110, 120, 130, 140, 150, 160, 170, 180, 190, 200, or more than 200) sample-indexed libraries can be pooled. Exemplary nucleic acid sources include, for example and without limitation, naturally occurring, recombinant, or chemically-synthesized sources. Exemplary nucleic acid sources include, for example and without limitation, single cells, a plurality of cells, tissue, biological fluid, an environmental sample, or a whole organism. Exemplary nucleic acid sources include, for example and without limitation, fresh, frozen, fresh-frozen or archived sources (e.g., formalin-fixed paraffin-embedded (FFPE)). The skilled artisan will recognize that the nucleic acids can be isolated from many other sources. In some embodiments, the nucleic acid library molecules can be prepared in single-stranded or double-stranded form.

In some embodiments, in the methods for forming a plurality of library-splint complexes (500), the nucleic acid library molecule (100) further comprises a first left unique identification sequence (180). In some embodiments, the nucleic acid library molecule (100) further comprises a first right unique identification sequence (190). In some embodiments, the first left unique identification sequence (180) and the first right unique identification sequence (190) each comprise a sequence that is used to uniquely identify an individual sequence of interest (e.g., insert sequence) to which the unique adaptors are appended in a population of other sequence of interest molecules. In some embodiments, the first left unique identification sequence (180) and/or the first right unique identification sequence (190) can be used for molecular tagging.

In some embodiments, in the methods for forming a plurality of library-splint complexes (500), the nucleic acid library molecule (100) comprises any of: a first left universal adaptor sequence (120); a second left universal adaptor sequence (140); a first left index sequence (160); a first left unique identification sequence (180); a first right universal adaptor sequence (130); a second right universal adaptor sequence (150); a first right index sequence (170); and/or a first right unique identification sequence (190). In some embodiments, in the methods for forming a plurality of library-splint complexes (500), the nucleic acid library molecule (100) comprises any combination of two or more of: a first left universal adaptor sequence (120); a second left universal adaptor sequence (140); a first left index sequence (160); a first left unique identification sequence (180); a first right universal adaptor sequence (130); a second right universal adaptor sequence (150); a first right index sequence (170); and/or a first right unique identification sequence (190).

In some embodiments, in the methods for forming a plurality of library-splint complexes (500), the first left universal adaptor sequence (120) and/or the second left universal adaptor sequence (140), comprises: a universal binding sequence for a forward or reverse sequencing primer; a universal binding sequence for a first or second surface primer; a universal binding sequence for a forward or reverse amplification primer; and/or a universal binding sequence for a compaction oligonucleotide. In some embodiments, the nucleic acid library molecule (100) can further comprise additional left universal adaptor sequences.

In some embodiments, in the methods for forming a plurality of library-splint complexes (500), the first right universal adaptor sequence (130) and/or the second right universal adaptor sequence (150), comprises: a universal binding sequence for a forward or reverse sequencing primer; a universal binding sequence for a first or second surface primer; a universal binding sequence for a forward or reverse amplification primer; or a universal binding sequence for a compaction oligonucleotide. In some embodiments, in the methods for forming a plurality of library-splint complexes (500), the first right universal adaptor sequence (130) and/or the second right universal adaptor sequence (150), comprises any combination of two or more of: a universal binding sequence for a forward or reverse sequencing primer; a universal binding sequence for a first or second surface primer; a universal binding sequence for a forward or reverse amplification primer; or a universal binding sequence for a compaction oligonucleotide. In some embodiments, the nucleic acid library molecule (100) can further comprise additional right universal adaptor sequences.

In some embodiments, in the methods for forming a plurality of library-splint complexes (500), the second splint strand (400) comprises at least two sub-regions, including a first and second sub-region (e.g., FIG. 1). In some embodiments, the first sub-region comprises a universal binding sequence for a third surface primer, and the second sub-region comprises a universal binding sequence for a fourth surface primer. In certain embodiments, the first and second sub-regions do not hybridize (e.g., exhibit very little hybridization or no hybridization to) the first and second surface primers. In some embodiments, the second splint strand (400) further comprises an optional third sub-region which includes a sample index sequence having 5-20 bases (e.g., about 5, 6, 7, 8, 9, 10, 11, 12, 13, 14, 15, 16, 17, 18, 19, or bases) and/or a unique identification sequence having 2-10 or more (e.g., about 2, 3, 4, 5, 6, 7, 8, 9, 10, or more than 10) bases (e.g., NN) (e.g., FIG. 1). In some embodiments, the second splint strand (400) comprises only one sub-region and lacks a second and third sub-region. In certain embodiments, the first sub-region comprises a sample index sequence having 5-20 bases (e.g., about 5, 6, 7, 8, 9, 10, 11, 12, 13, 14, 15, 16, 17, 18, 19, or 20 bases). In some embodiments, the sample index sequence can be used to distinguish sequences of interest obtained from different sample sources in a multiplex assay. In some embodiments, the unique identification sequence comprises a random sequence. In some embodiments, the unique identification sequence can be designed to exhibit reduced or no hybridization to the first, second, third and fourth surface primers. A non-limiting exemplary arrangement of the sub-regions in the second splint strand (400), in a 5′ to 3′ orientation comprises: 5′-[second sub-region]-[first sub-region]-3′. Another non-limiting exemplary arrangement of the sub-regions in the second splint strand (400), in a 5′ to 3′ orientation comprises: 5′-[third sub-region]-[second sub-region]-[first sub-region]-3′. In some embodiments, the second splint strand (400) can be 20-100 (e.g., about 20, 25, 30, 35, 40, 45, 50, 60, 70, 80, 90, or 100) nucleotides in length. In some embodiments, the second splint strand (400) can be 30-80 (e.g., about 30, 35, 40, 45, 50, 60, 70, or 80) nucleotides in length. In some embodiments, the second splint strand (400) can be 40-60 (e.g., about 40, 45, 50, or 60) nucleotides in length. In some embodiments, the second splint strands (400) comprise one or more phosphorothioate linkage at the 5′ and/or 3′ ends to confer exonuclease resistance. In some embodiments, the second splint strands (400) comprise one or more phosphorothioate linkages at an internal position to confer endonuclease resistance. In some embodiments, the second splint strands (400) comprise one or more 2′-O-methylcytosine bases at the 5′ and/or 3′ end, or at an internal position. In some embodiments, the 5′ end of the second splint strand (400) is phosphorylated. In some embodiments, the 5′ end of the second splint strand (400) is non-phosphorylated. In some embodiments, the 3′ end of the second splint strand (400) comprises a terminal 3′ OH group or a terminal 3′ blocking group.

In some embodiments, in the methods for forming a plurality of library-splint complexes (500), the first splint strand (300) includes an internal region (310) which comprises at least two sub-regions, including a fourth and fifth sub-region (e.g., FIG. 1). In some embodiments, the fourth sub-region hybridizes to the first sub-region of the second splint strand (400). In some embodiments, the fifth sub-region hybridizes to the second sub-region of the second splint strand (400). In some embodiments, the fourth and fifth sub-regions do not hybridize (e.g., exhibit very little or no hybridization to) the first and second surface primers. In some embodiments, the internal region (310) of the first splint strand further comprises an optional sixth sub-region which hybridizes to the third sub-region of the second splint strand (400) (e.g., FIG. 1). A non-limiting exemplary arrangement of the sub-regions of the first splint strand (300), in a 3′ to 5′ orientation comprises: 3′-[fourth sub-region]-[fifth sub-region]-5′. Another non-limiting exemplary arrangement of the sub-regions of the first splint strand (300), in a 3′ to 5′ orientation comprises: 3′-[fourth sub-region]-[fifth sub-region]-[sixth sub-region]-5′. In some embodiments, the first splint strand (300) can be 50-150 (e.g., about 50, 60, 70, 80, 90, 100, 110, 120, 130, 140, or 150) nucleotides in length. In some embodiments, the first splint strand (300) can be 60-100 (e.g., about 60, 70, 80, 90, or 100) nucleotides in length. In some embodiments, the first splint strand (300) can be 70-90 (e.g., about 70, 75, 80, 85 or 90) nucleotides in length. In some embodiments, the first splint strands (300) comprise one or more phosphorothioate linkages at the 5′ and/or 3′ ends to confer exonuclease resistance. In some embodiments, the first splint strands (300) comprise one or more phosphorothioate linkages at an internal position to confer endonuclease resistance. In some embodiments, the first splint strands (300) comprise one or more 2′-O-methylcytosine bases at the 5′ and/or 3′ end, or at an internal position.

In another aspect, the present disclosure provides methods for forming a plurality of library-splint complexes (500) comprising: (a) providing a plurality of double-stranded splint adaptors (200) wherein individual double-stranded splint adaptors (200) comprise a first splint strand (300) hybridized to a second splint strand (400). In some embodiments, the first splint strand (300) comprises regions arranged in a 5′ to 3′ order a first region (320), an internal region (310), and a second region (330). In some embodiments, the internal region of the first splint strand (310) is hybridized to the second splint strand (400). In certain embodiments, the second splint strand comprises regions arranged in a 5′ to 3′ order: (i) a second sub-region having a universal binding sequence for a fourth surface primer, and (ii) a first sub-region having a universal binding sequence for a third surface primer. In some embodiments, the methods for forming a plurality of library-splint complexes (500) further comprises step (b): hybridizing the plurality of double-stranded splint adaptors with a plurality of single-stranded nucleic acid library molecules (100). In certain embodiments, the individual library molecules comprise regions arranged in a 5′ to 3′ order: (i) a first left universal adaptor sequence (120) having a binding sequence for a first surface primer; (ii) a second left universal adaptor sequence (140) having a binding sequence for a first sequencing primer; (iii) a sequence of interest (110); (iv) a second right universal adaptor sequence (150) having a binding sequence for a second sequencing primer; and (v) a first right universal adaptor sequence (130) having a binding sequence for a second surface primer (130). In certain embodiments, the hybridizing is conducted under a condition suitable to hybridize the first splint strand (300) to the library molecule (100), thereby circularizing the library molecule to generate a library-splint complex (500). In some embodiments, the first region (320) of the first splint strand is hybridized to the binding sequence for the first surface primer (120), and the third region (330) of the first splint strand is hybridized to the binding sequence for the second surface primer (130). In certain embodiments, the library-splint complex (500) comprises a first nick between the 5′ end of the library molecule and the 3′ end of the second splint strand (300). In certain embodiments, the library-splint complex (500) comprises a second nick between the 5′ end of the second splint strand (300) and the 3′ end of the library molecule (100). In certain embodiments, the first and second nicks are enzymatically ligatable. In some embodiments, the plurality of single-stranded nucleic acid library molecules (100) further comprises a first left index sequence (160) and/or a first right index sequence (170) (e.g., see FIGS. 1 and 2). A list of exemplary first left index sequences (160) and first right index sequences (170) is provided in Tables 1-2 at FIG. 7. In some embodiments, the first left index sequences (160) include a short random sequence (e.g., NNN). In some embodiments, the first left index sequences (160) lack a short random sequence (e.g., NNN). In some embodiments, the first right index sequences (170) include a short random sequence (e.g., NNN). In some embodiments, the first right index sequences (170) lack a short random sequence (e.g., NNN). In some embodiments, the plurality of single-stranded nucleic acid library molecules (100) further comprises a first left unique identification sequence (180) and/or a first right unique identification sequence (190). In certain embodiments, each unique identification sequence comprises a sequence that is used to uniquely identify an individual sequence of interest (e.g., insert sequence) to which the unique adaptors are appended in a population of other sequence of interest molecules. In some embodiments, the first left unique identification sequence (180) and/or the first right unique identification sequence (190) can be used for molecular tagging. (e.g., see FIG. 1).

Multiplex workflows are enabled by preparing sample-indexed libraries using one or both index sequences (e.g., left and/or right index sequences). In some embodiments, the first left index sequences (160) and/or first right index sequences (170) can be employed to prepare separate sample-indexed libraries using input nucleic acids isolated from different sources. In some embodiments, the sample-indexed libraries can be pooled together to generate a multiplex library mixture, and the pooled libraries can be amplified and/or sequenced. In some embodiments, the sequences of the insert region along with the first left index sequence (160) and/or first right index sequence (170) can be used to identify the source of the input nucleic acids. In some embodiments, any number of sample-indexed libraries can be pooled together, for example 2-10 (e.g., about 2, 3, 4, 5, 6, 7, 8, 9, or 10) libraries. In some embodiments, 10-50 (e.g., about 10, 20, 30, 40, or 50) sample-indexed libraries are pooled together. In some embodiments, 50-100 (e.g., about 50, 60, 70, 80, 90, or 100) sample-indexed libraries are pooled together. In some embodiments, 100-200 (e.g., about 100, 110, 120, 130, 140, 150, 160, 170, 180, 190, or 200) sample-indexed libraries are pooled together. In some embodiments, more than 200 sample-indexed libraries can be pooled. Exemplary nucleic acid sources include, without limitation, naturally occurring, recombinant, or chemically synthesized sources. Exemplary nucleic acid sources include, without limitation, single cells, a plurality of cells, tissue, biological fluid, an environmental sample, or a whole organism. Exemplary nucleic acid sources include, without limitation, fresh, frozen, fresh-frozen, or archived sources (e.g., formalin-fixed paraffin-embedded; FFPE). The skilled artisan will recognize that the nucleic acids can be isolated from many other sources. In some embodiments, the nucleic acid library molecules can be prepared in single-stranded or double-stranded form.

In some embodiments, the plurality of single-stranded nucleic acid library molecules (100) further comprises a first left unique identification sequence (180) and/or a first right unique identification sequence (190) (e.g., see FIG. 1). In some embodiments, the first left unique identification sequence (180) and the first right unique identification sequence (190) each comprise a sequence that is used to uniquely identify an individual sequence of interest (e.g., insert sequence) to which the unique adaptors are appended in a population of other sequence of interest molecules. In some embodiments, the first left unique identification sequence (180) and/or the first right unique identification sequence (190) can be used for molecular tagging.

In some embodiments, any of the methods for forming a plurality of library-splint complexes (500) described herein can further comprise at least one enzymatic reaction, e.g., a phosphorylation reaction, ligation reaction, exonuclease reaction, or a combination thereof. The enzymatic reactions can be conducted sequentially or essentially simultaneously. In some embodiments, the enzymatic reactions can be conducted in a single reaction vessel. Alternatively, in some embodiments, a first enzymatic reaction can be conducted in a first reaction vessel, then transferred to a second reaction vessel where the second enzymatic reaction is conducted, then transferred to a third reaction vessel where the third enzymatic reaction is conducted, etc.

In some embodiments, any of the methods for forming a plurality of library-splint complexes (500) described herein further comprise conducting separate and sequential phosphorylation and ligation reactions which are conducted in separate reaction vessels. In some embodiments, the methods for forming a plurality of library-splint complexes (500) further comprise step (el): contacting in a first reaction vessel the plurality of the double-stranded splint adaptors (200) and the plurality of the single-stranded nucleic acid library molecules (100) with a T4 polynucleotide kinase enzyme under a condition suitable to phosphorylate the 5′ ends of the plurality of double-stranded splint adaptors (200) and/or the plurality of single-stranded nucleic acid library molecules (100). In some embodiments, the phosphorylation reaction is transferred to a second reaction vessel. In some embodiments, the methods for forming a plurality of library-splint complexes (500) further comprise step (d1): contacting in the second reaction vessel the plurality of phosphorylated double-stranded splint adaptors (200) and the plurality of phosphorylated single-stranded nucleic acid library molecules (100) with a ligase, under a condition suitable to enzymatically ligate the first and second nicks, thereby generating a plurality of covalently closed circular library molecules (600) each hybridized to the first splint strand (300). In some embodiments, the ligase enzyme comprises T7 DNA ligase, T3 ligase, T4 ligase, or Taq ligase.

In some embodiments, any of the methods for forming a plurality of library-splint complexes (500) described herein further comprise conducting sequential phosphorylation and ligation reactions which are conducted sequentially in the same reaction vessel. In some embodiments, the methods for forming a plurality of library-splint complexes (500) further comprise step (c2): contacting in a first reaction vessel the plurality of the double-stranded splint adaptors (200) and the plurality of the single-stranded nucleic acid library molecules (100) with a T4 polynucleotide kinase enzyme under a condition suitable to phosphorylate the 5′ ends of the plurality of double-stranded splint adaptors (200) and the plurality of single-stranded nucleic acid library molecules (100). In some embodiments, the methods for forming a plurality of library-splint complexes (500) further comprise step (d2): contacting in the same first reaction vessel the phosphorylated double-stranded splint adaptors (200) and the phosphorylated single-stranded nucleic acid library molecules (100) with a ligase under a condition suitable to enzymatically ligate the first and second nicks, thereby generating a plurality of covalently closed circular library molecules (600) each hybridized to the first splint strand (300). In some embodiments, the ligase enzyme comprises T7 DNA ligase, T3 ligase, T4 ligase, or Taq ligase.

In some embodiments, any of the methods for forming a plurality of library-splint complexes (500) described herein further comprise conducting essentially simultaneous phosphorylation and ligation reactions which are conducted together in the same reaction vessel. In some embodiments, the methods for forming a plurality of library-splint complexes (500) further comprise step (c3): contacting in a first reaction vessel the plurality of the double-stranded splint adaptors (200) and the plurality of the single-stranded nucleic acid library molecules (100) with a (i) T4 polynucleotide kinase enzyme and (ii) a ligase enzyme, under a condition suitable to phosphorylate the 5′ ends of the plurality of double-stranded splint adaptors (200) and the plurality of single-stranded nucleic acid library molecules (100). In some embodiments, the conditions are suitable to enzymatically ligate the first and second nicks, thereby generating a plurality of covalently closed circular library molecules (600) each hybridized to the first splint strand (300). In some embodiments, the ligase enzyme comprises T7 DNA ligase, T3 ligase, T4 ligase, or Taq ligase.

In some embodiments, any of the methods for forming a plurality of library-splint complexes (500) described herein further comprise the optional step of enzymatically removing the plurality of first splint strands (300) from the plurality of covalently closed circular library molecules (600), which comprises: contacting the plurality of covalently closed circular library molecules (600) with at least one exonuclease enzyme to remove the plurality of first splint strands (300) and retaining the plurality of covalently closed circular library molecules (600). In some embodiments, the exonuclease reaction can be conducted in the same reaction buffer used to conduct the phosphorylation and/or ligation reactions. In some embodiments, the exonuclease reaction can be conducted in a different reaction buffer than that used to conduct the phosphorylation and/or ligation reactions. In some embodiments, the exonuclease reaction can be conducted in a third reaction vessel after conducting the phosphorylation reaction in the first reaction vessel (c1) and conducting the ligation reaction in the second reaction vessel (d1). In some embodiments, the exonuclease reaction can be conducted in the first reaction vessel after conducting the phosphorylation reaction in the first reaction vessel (c2) and conducting the sequential ligation reaction in the first reaction vessel (d2). In some embodiments, the exonuclease reaction can be conducted in the first reaction vessel after conducting the essentially simultaneous phosphorylation and ligation reactions in the first reaction vessel (c3). In some embodiments, the at least one exonuclease enzyme comprises any combination of two or more of exonuclease I, thermolabile exonuclease I, and/or T7 exonuclease.

In some embodiments, covalently closed circular library molecules (600) can be generated using any of the methods described above. In some embodiments, individual covalently closed circular library molecules comprise a sequence of interest operably linked on both sides by at least one nucleic acid adaptor sequence. In some embodiments, at least one covalently closed circular library molecule carries at least one deaminated nucleotide base. In some embodiments, the covalently closed circular library molecules can be treated with a reagent that removes deaminated bases. In some embodiments, the reagent that removes deaminated bases comprises a compound that generates abasic sites at uracil bases in a nucleic acid molecule. For example, and without limitation, DNA glycosylase (UDG) can generate abasic sites at uracil bases. In some embodiments, the reagent that removes deaminated bases comprises a compound that generates a gap at abasic sites in a nucleic acid strand. For example, and without limitation, the gaps can be generated with an enzyme or a mixture of enzymes having lyase activity that breaks the phosphodiester backbone at the 5′ and 3′ sides of the abasic site to release the base-free deoxyribose and generate a gap. The abasic sites can be removed using, for example and without limitation, AP lyase, Endo IV endonuclease, FPG glycosylase/AP lyase, Endo VIII glycosylase/AP lyase, and combinations thereof. In some embodiments, generating the abasic sites and removal of the abasic sites to generate gaps can be achieved using a mixture of uracil DNA glycosylase and DNA glycosylase-lyase endonuclease VIII. Suitable uracil DNA glycosylase and DNA glycosylase-lyase endonuclease VIII include, for example and without limitation, USER™ (Uracil-Specific Excision Reagent Enzyme from New England Biolabs™) or thermolabile USER™ (also from New England Biolabs™).

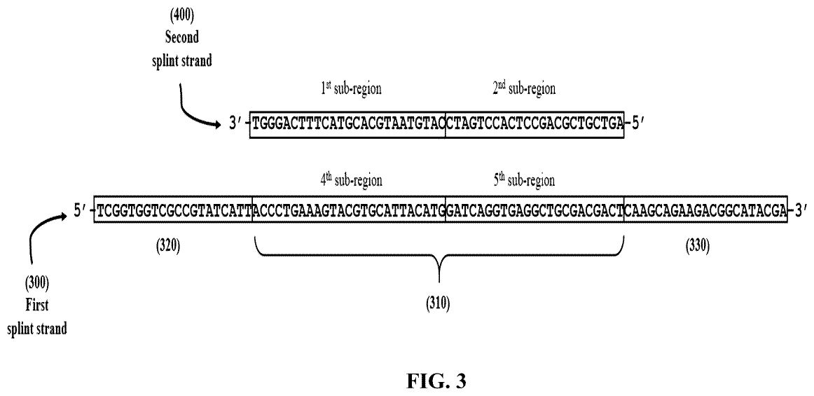

In some embodiments, in any of the methods for forming a plurality of library-splint complexes (500) described herein, the first sub-region of the second splint strand (400) comprises the sequence 5′-CATGTAATGCACGTACTTTCAGGGT-3′ (SEQ ID NO:1). In some embodiments, the second sub-region of the second splint strand (400) comprises the sequence 5′-AGTCGTCGCAGCCTCACCTGATC-3′ (SEQ ID NO:2). In some embodiments, the second splint strand (400) comprises a first and second sub-region comprising the sequence ‘-AGTC GTC GCAGC C TCAC C TGATC C ATGTAATGC AC GTACTTTC AGGGT-3’ (SEQ ID NO:3). See FIG. 3. In some embodiments, the 5′ end of the second splint strand (400) can be phosphorylated. In some embodiments, the 5′ end of the second splint strand (400) can be non-phosphorylated.

In some embodiments, in any of the methods for forming a plurality of library-splint complexes (500) described herein, the first region of the first splint strand (320) includes a first universal adaptor sequence which comprises a universal binding sequence (or a complementary sequence thereof) for a first surface primer. In some embodiments, the first region (320) comprises the sequence 5′-TCGGTGGTCGCCGTATCATT-3′ (SEQ ID NO:4). For example, and without limitation, the first region of the first splint strand (320) can hybridize to a P5 surface primer or a complementary sequence of the P5 surface primer. For example and without limitation, the P5 surface primer comprises the sequence AATGATACGGCGACCACCGA-3′ (SEQ ID NO:204; short P5), or the P5 surface primer comprises the sequence 5′-AATGATACGGCGACCACCGAGATC-3′ (SEQ ID NO:205; long P5). In some embodiments, the second region of the first splint strand (330) includes a second universal adaptor sequence which comprises a universal binding sequence (or a complementary sequence thereof) for a second surface primer. In some embodiments, the second region (330) comprises the sequence 5′-CAAGCAGAAGACGGCATACGA-3′ (SEQ ID NO:5). For example, and without limitation, the second region of the first splint strand (330) can hybridize to a P7 surface primer or a complementary sequence of the P7 surface primer. For example, and without limitation, the P7 surface primer comprises the sequence 5′-CAAGCAGAAGACGGCATACGA-3′ (SEQ ID NO:5; short P7), or the P7 surface primer comprises the sequence 5′-CAAGCAGAAGACGGCATACGAGAT-3′ (SEQ ID NO:206; long P7). In some embodiments, the first splint strand (300) includes an internal region (310) which comprises a fourth sub-region having the sequence (SEQ ID NO:6). In some embodiments, the first splint strand (300) includes an internal region (310) which comprises a fifth sub-region having the sequence 5′-GATCAGGTGAGGCTGCGACGACT-3′ (SEQ ID NO:7). In some embodiments, the first splint strand (300) comprises a first region (320), an internal region (310) having a fourth and fifth sub-region, and a second region (330), having the sequence 5′-TCGGTGGTCGCCGTATCATTACCCTGAAAGTACGTGCATTACATGGATCAGGTGA GGCTGCGACGACTCAAGCAGAAGACGGCATACGA-3′ (SEQ ID NO:8). See FIG. 3. In some embodiments, the 5′ end of the first splint strand (300) can be phosphorylated. In some embodiments, the 5′ end of the first splint strand (300) can be non-phosphorylated. In some embodiments, the first sub-region of the second splint strand (400) can hybridize to the fourth sub-region of the first splint strand (300). In some embodiments, the second sub-region of the second splint strand (400) can hybridize to the fifth sub-region of the first splint strand (300).

In some embodiments, in any of the methods for forming a plurality of library-splint complexes (500) described herein, the first region of the first splint strand (320) comprises a sequence that can bind a first left universal adaptor sequence (120) of a library molecule, where the first region of the first splint strand (320) comprises the sequence 5′-ACCCTGAAAGTACGTGCATTACATG-3′ (SEQ ID NO:6) or a complementary sequence thereof.

In some embodiments, in any of the methods for forming a plurality of library-splint complexes (500) described herein, the second region of the first splint strand (330) comprises a sequence that can bind a first right universal adaptor sequence (130) of a library molecule, where the second region of the first splint strand (330) comprises the sequence 5′-GATCAGGTGAGGCTGCGACGACT-3′ (SEQ ID NO:7) or a complementary sequence thereof.

In some embodiments, in any of the methods for forming a plurality of library-splint complexes (500) described herein, the library molecule includes a left universal binding sequence (120) which binds the first region of the first splint strand (320), where the left universal binding sequence (120) comprises the sequence AATGATACGGCGACCACCGA-3′ (SEQ ID NO:204).

In some embodiments, in any of the methods for forming a plurality of library-splint complexes (500) described herein, the library molecule includes a left universal binding sequence (120) which binds the first region of the first splint strand (320), where the left universal binding sequence (120) comprises the sequence 5′-CATGTAATGCACGTACTTTCAGGGT-3′ (SEQ ID NO:1) or a complementary sequence thereof.

In some embodiments, in any of the methods for forming a plurality of library-splint complexes (500) described herein, the library molecule includes a left universal binding sequence for a sequencing primer (140) where the left universal binding sequence comprises the sequence 5′-ACACTCTTTCCCTACACGACGCTCTTCCGATCT-3′ (SEQ ID NO:207).

In some embodiments, in any of the methods for forming a plurality of library-splint complexes (500) described herein, the library molecule includes a left universal binding sequence for a sequencing primer (140) where the left universal binding sequence comprises the sequence 5′-TCGTCGGCAGCGTCAGATGTGTATAAGAGACAG-3′ (SEQ ID NO:208).

In some embodiments, in any of the methods for forming a plurality of library-splint complexes (500) described herein, the library molecule includes a left universal binding sequence for a sequencing primer (140) where the left universal binding sequence comprises the sequence 5′-CGTGCTGGATTGGCTCACCAGACACCTTCCGACAT-3′ (SEQ ID NO:209).

In some embodiments, in any of the methods for forming a plurality of library-splint complexes (500) described herein, the library molecule includes a left universal binding sequence for a sequencing primer (150) where the left universal binding sequence comprises the sequence 5′-AGATCGGAAGAGCACACGTCTGAACTCCAGTCAC-3′ (SEQ ID NO:210).

In some embodiments, in any of the methods for forming a plurality of library-splint complexes (500) described herein, the library molecule includes a left universal binding sequence for a sequencing primer (150) where the left universal binding sequence comprises the sequence 5′-CTGTCTCTTATACACATCTCCGAGCCCACGAGAC-3′ (SEQ ID NO:211).

In some embodiments, in any of the methods for forming a plurality of library-splint complexes (500) described herein, the library molecule includes a left universal binding sequence for a sequencing primer (150) where the left universal binding sequence comprises the sequence 5′-ATGTCGGAAGGTGTGCAGGCTACCGCTTGTCAACT-3′ (SEQ ID NO:212).

In some embodiments, in any of the methods for forming a plurality of library-splint complexes (500) described herein, the library molecule includes a right universal binding sequence (130) which binds the first region of the first splint strand (330), where the right universal binding sequence (130) comprises the sequence TCGTATGCCGTCTTCTGCTTG-3′ (SEQ ID NO:213).

In some embodiments, in any of the methods for forming a plurality of library-splint complexes (500) described herein, the library molecule includes a right universal binding sequence (130) which binds the first region of the first splint strand (330), where the right universal binding sequence (130) comprises the sequence 5′-AGTCGTCGCAGCCTCACCTGATC-3′ (SEQ ID NO:2) or a complementary sequence thereof

### Methods for Rolling Circle Amplification

### Using Circularized Library Molecules Generated Via Ds-Splint Adaptors

The present disclosure provides methods for conducting a rolling circle amplification reaction on the covalently closed circular library molecules (600). In some embodiments, the rolling circle amplification reaction can be conducted after both the phosphorylation and ligation reactions. In some embodiments, the rolling circle amplification reaction is after the ligation reaction. In some embodiments, the rolling circle amplification reaction can be conducted on covalently closed circular library molecules (600) that are no longer hybridized to the first splint strands (300) following the exonuclease reaction. In some embodiments, the rolling circle amplification reaction can be conducted on covalently closed circular library molecules (600) that are hybridized to the first splint strands (300). In some embodiments, the covalently closed circular library molecules (600) can be distributed onto a support and then be subjected to rolling circle amplification reaction. In some embodiments, the covalently closed circular library molecules (600) can be subjected to rolling circle amplification reaction in-solution and then distributed onto a support. In some embodiments, the rolling circle amplification reactions can employ the retained first splint strand (300) as an amplification primer, or the first splint strand (300) can be removed (e.g., via exonuclease digestion) and replaced with a soluble amplification primer.

### On-Support Rolling Circle Amplification Using Circularized Library Molecules Generated Via Ds-Splint Adaptors

In some embodiments, the methods for conducting rolling circle amplification reaction on a plurality of covalently closed circular library molecules which lack hybridized first splint strands (300). In certain embodiments, individual covalently closed circular library molecules (600) in the plurality comprise a second splint strand region (400) which includes a universal binding sequence for a third surface primer, the method comprises (a): distributing the plurality of covalently closed circular library molecules (600) onto a support having a plurality of the third surface primers immobilized on the support, under a condition suitable for hybridizing individual covalently closed circular library molecules (600) to individual immobilized third surface primers thereby immobilizing the plurality of covalently closed circular library molecules (600).

In some embodiments, the plurality of the third surface primers immobilized on the support comprise the sequence 5′-GATCAGGTGAGGCTGCGACGACT-3′ (SEQ ID NO:7). Individual third surface primers can hybridize to a covalently closed circular library molecule (600) having a second splint strand region (400) which includes a universal binding sequence for a third surface primer. In certain embodiments, the universal binding sequence for a third surface primer comprises a second sub-region which comprises the sequence 5′-AGTCGTCGCAGCCTCACCTGATC-3′ (SEQ ID NO:2).

In some embodiments, the plurality of covalently closed circular library molecules (600) can be distributed onto a support that is coated with one or more compounds to produce a passivated layer on the support (e.g., FIG. 8). In some embodiments, the passivated layer forms a porous layer. In some embodiments, the passivated layer forms a semi-porous layer. In some embodiments, the surface primer, concatemer template molecule, polymerase, or a combination thereof, can be attached to the passivated layer for immobilization to the support. In some embodiments, the support comprises a low non-specific binding surface that enables improved nucleic acid hybridization and amplification performance on the support. In general, the support may comprise one or more layers of a covalently or non-covalently attached low-binding, chemical modification layers, e.g., silane layers, polymer films, and one or more covalently or non-covalently attached oligonucleotides that can be used for immobilizing a plurality of nucleic acid concatemer molecules to the support. In some embodiments, the support can comprise a functionalized polymer coating layer covalently bound at least to a portion of the support, e.g., via a chemical group on the support, a primer grafted to the functionalized polymer coating, or a water-soluble protective coating on the primer and the functionalized polymer coating. In some embodiments, the functionalized polymer coating comprises a poly(N-(5-azidoacetamidylpentyl)acrylamide-co-acrylamide (PAZAM). In some embodiments, the support comprises a surface coating having at least one hydrophilic polymer coating layer and at least one layer of a plurality of oligonucleotides. In certain embodiments, the hydrophilic polymer coating layer can comprise a polyethylene glycol (PEG). In certain embodiments, the hydrophilic polymer coating layer can comprise a branched PEG having at least 4 branches. In some embodiments, the low non-specific binding coating has a degree of hydrophilicity which can be measured as a water contact angle. In certain embodiments, the water contact angle is no more than 45 degrees (e.g., no more than 5 degrees, no more than 10 degrees, no more than 15 degrees, no more than 20 degrees, no more than 25 degrees, no more than 30 degrees, no more than 35 degrees, no more than 40 degrees, or no more than 45 degrees). In some embodiments, the density of the covalently closed circular library molecules (600) immobilized to the support or immobilized to the coating on the support is about 10²-10⁶ per mm² (e.g., about 10², 10³, 10⁴, 10⁵, or 10⁶). In some embodiments, the density of the covalently closed circular library molecules (600) immobilized to the support or immobilized to the coating on the support is about 10⁶-10⁹ per mm² (e.g., about 10⁶, 10⁷, 10⁸, or 10⁹). In some embodiments, the density of the covalently closed circular library molecules (600) immobilized to the support or immobilized to the coating on the support is about 10⁹-10¹² (e.g., about 10⁹, 10¹⁰, 10¹¹, or 10¹²) per mm². In some embodiments, the plurality of covalently closed circular library molecules (600) is immobilized to the support or immobilized to the coating on the support at pre-determined sites on the support (or the coating on the support). In some embodiments, the plurality of covalently closed circular library molecules (600) is immobilized to the support or immobilized to the coating on the support at random sites on the support (or the coating on the support).

In some embodiments, the distributing of step (a) can be conducted in the presence of a high-efficiency hybridization buffer which comprises: (i) a first polar aprotic solvent having a dielectric constant that is no greater than 40 (e.g., less than 10, or about 10, 15, 20, or 40) and having a polarity index of 4-9 (e.g., 4, 5, 6, 7, 8, or 9); (ii) a second polar aprotic solvent having a dielectric constant that is no greater than 115 (e.g., less than 10, 10, 15, 20, 40, 50, 75, 100, 105, 105, 110, or 115) and is present in the hybridization buffer formulation in an amount effective to denature double-stranded nucleic acids; (iii) a pH buffer system that maintains the pH of the hybridization buffer formulation in a range of about 4-8 (e.g., 4, 5, 6, 7, or 8); and (iv) a crowding agent in an amount sufficient to enhance or facilitate molecular crowding. In some embodiments, the high efficiency hybridization buffer comprises: (i) the first polar aprotic solvent comprises acetonitrile at 25-50% (e.g., 25%, 30%, 35%, 40%, 45%, or 50%) by volume of the hybridization buffer; (ii) the second polar aprotic solvent comprises formamide at 5-10% (e.g., 5%, 6%, 7%, 8%, 9%, or 10%) by volume of the hybridization buffer; (iii) the pH buffer system comprises 2-(N-morpholino)ethanesulfonic acid (MES) at a pH of 5-6.5 (e.g., about 5.0, 5.5, 6.0, or 6.5); and (iv) the crowding agent comprises polyethylene glycol (PEG) at 5-35% (e.g., 5%, 10%, 15%, 20%, 25%, 30%, or 35%) by volume of the hybridization buffer. In some embodiments, the high efficiency hybridization buffer further comprises betaine.

In some embodiments, the methods for conducting rolling circle amplification reaction further comprises step (b): contacting the plurality of immobilized covalently closed circular library molecules (600) with a plurality of strand-displacing polymerases and a plurality of nucleotides (e.g., comprising bases A, G, C, T and/or U), under a condition suitable to conduct a rolling circle amplification reaction on the support using the plurality of third surface primers as immobilized amplification primers and the plurality of covalently closed circular library molecules (600) as template molecules, thereby generating a plurality of nucleic acid concatemer molecules immobilized to the third surface primers. In some embodiments, the plurality of nucleotides comprises any combination of two or more of dATP, dGTP, dCTP, dTTP and/or dUTP. In some embodiments, individual immobilized concatemers are covalently joined to individual third surface primers. In some embodiments, individual covalently closed circular library molecules (600) in the plurality comprise a second splint strand region (400) which also include a universal binding sequence for a fourth surface primer so that the rolling circle amplification reaction generates concatemer molecules having multiple copies of universal binding sequences for third and fourth surface primers. In some embodiments, the method comprises distributing the covalently closed circular library molecules (600) onto a support comprising a plurality of immobilized third and fourth surface primers, conducting a rolling circle amplification reaction to generate concatemer molecules under a condition suitable for hybridizing at least one second splint strand region (400) of the concatemer molecules to immobilized third and fourth surface primers thereby pinning down at least one portion of the concatemer molecules to the support. In some embodiments, the immobilized concatemers can be subjected to sequencing reactions.

In some embodiments, the plurality of the fourth surface primers immobilized on the support comprise the sequence 5′-CATGTAATGCACGTACTTTCAGGGT-3′ (SEQ ID NO:1) or a complementary sequence thereof). Individual fourth surface primers can hybridize to a portion of the concatemer molecules having a second splint strand region (400) which includes a universal binding sequence for a fourth surface primer (or a complementary sequence thereof), where the universal binding sequence for the fourth surface primer comprises a first sub-region which comprises the sequence 5′-CATGTAATGCACGTACTTTCAGGGT-3′ (SEQ ID NO:1).

### In-Solution Rolling Circle Amplification Using Soluble Amplification Primers Using Circularized Library Molecules Generated Via Ds-Splint Adaptors

In some embodiments, the methods for conducting rolling circle amplification reaction on a plurality of covalently closed circular library molecules which lack hybridized first splint strands (300), wherein individual covalently closed circular library molecules (600) in the plurality comprise a first (120) or second (140) left universal adaptor sequence of a universal binding sequence for a forward amplification primer. In some embodiments, individual covalently closed circular library molecules (600) in the plurality comprise a second splint strand region (400) which includes a universal binding sequence for a third surface primer. In some embodiments, the method comprises (a) hybridizing in solution a plurality of soluble forward amplification primers to the first or second left universal adaptor sequence which comprise the universal binding sequence for a forward amplification primer; and (b) conducting a first rolling circle amplification reaction by contacting the plurality of covalently closed circular library molecules (600) with a plurality of strand-displacing polymerases and a plurality of nucleotides (e.g., comprising bases A, G, C, T and/or U), under a condition suitable to conduct a rolling circle amplification reaction in solution using the plurality of forward amplification primers and the plurality of covalently closed circular library molecules (600) as template molecules, thereby generating a plurality of nucleic acid concatemer molecules. In certain embodiments, the nucleic acid concatemer molecules are still hybridized to covalently closed circular library molecules (600). In some embodiments, the methods for conducting rolling circle amplification reaction further comprise step (c): distributing the plurality of concatemer molecules onto a support having a plurality of the third surface primers immobilized thereon, under a condition suitable for hybridizing at least a portion of the concatemers to the plurality of the immobilized third surface primers, thereby immobilizing the plurality of concatemer molecules. In some embodiments, the plurality of immobilized concatemer molecules is still hybridized to covalently closed circular library molecules (600). In some embodiments, the methods for conducting rolling circle amplification reaction further comprise step (d): contacting the immobilized plurality of concatemer molecules with a plurality of strand-displacing polymerases and a plurality of nucleotides (e.g., comprising bases A, G, C, T and/or U), under a condition suitable to conduct a second rolling circle amplification reaction on the support using the plurality of covalently closed circular library molecules (600) as template molecules, thereby extending the plurality of immobilized nucleic acid concatemer molecules. In some embodiments, the first and/or the second rolling circle amplification reactions can be conducted with a plurality of nucleotides which comprise any combination of two or more of dATP, dGTP, dCTP, dTTP and/or dUTP. In some embodiments, individual immobilized concatemers are hybridized to individual third surface primers. In some embodiments, individual covalently closed circular library molecules (600) in the plurality comprise a second splint strand region (400) which also include a universal binding sequence for a fourth surface primer so that the first rolling circle amplification reaction in solution generates concatemer molecules having multiple copies of universal binding sequences for third and fourth surface primers. In some embodiments, the method comprises distributing the concatemer molecules onto a support comprising a plurality of immobilized third and fourth surface primers and incubating the concatemer molecules under a condition suitable for hybridizing at least one second splint strand region (400) of the concatemer molecules to immobilized third and fourth surface primers thereby pinning down at least one portion of the concatemer molecules to the support. In some embodiments, the immobilized concatemers can be subjected to sequencing reactions.

In some embodiments, the plurality of concatemer molecules of step (c) can be distributed onto a support comprising a plurality of the third surface primers immobilized on the support comprise the sequence 5′-GATCAGGTGAGGCTGCGACGACT-3′ (SEQ ID NO:7). In some embodiments, individual third surface primers can hybridize to a portion of a concatemer molecule having a universal binding sequence for a third surface primer, where the universal binding sequence for a third surface primer comprises the sequence 5′-AGTCGTCGCAGCCTCACCTGATC-3′ (SEQ ID NO:2 or a complementary sequence thereof).

In some embodiments, the plurality of concatemer molecules of step (c) can be distributed onto a support comprising a plurality of the fourth surface primers immobilized on the support comprise the sequence 5′-CATGTAATGCACGTACTTTCAGGGT-3′ (SEQ ID NO:1 or a complementary sequence thereof). In some embodiments, individual fourth surface primers can hybridize to a portion of the concatemer molecules having a universal binding sequence for a fourth surface primer (or a complementary sequence thereof), where the universal binding sequence for the fourth surface primer comprises the sequence 5′-CATGTAATGCACGTACTTTCAGGGT-3′ (SEQ ID NO:1).

In some embodiments, the plurality of concatemer molecules of step (c) can be distributed onto a support that is coated with one or more compounds to produce a passivated layer on the support (e.g., FIG. 8). In some embodiments, the passivated layer forms a porous or semi-porous layer. In some embodiments, the surface primer, concatemer template molecule and/or polymerase, can be attached to the passivated layer for immobilization to the support. In some embodiments, the support comprises a low non-specific binding surface that enables improved nucleic acid hybridization and amplification performance on the support. In general, the support may comprise one or more layers of a covalently or non-covalently attached low-binding, chemical modification layers, e.g., silane layers, polymer films, and one or more covalently or non-covalently attached oligonucleotides that can be used for immobilizing a plurality of nucleic acid concatemer molecules to the support. In some embodiments, the support can comprise a functionalized polymer coating layer covalently bound at least to a portion of the support via a chemical group on the support, a primer grafted to the functionalized polymer coating, and a water-soluble protective coating on the primer and the functionalized polymer coating. In some embodiments, the functionalized polymer coating comprises a poly(N-(5-azidoacetamidylpentyl)acrylamide-co-acrylamide (PAZAM). In some embodiments, the support comprises a surface coating having at least one hydrophilic polymer coating layer and at least one layer of a plurality of oligonucleotides. In some embodiments, the hydrophilic polymer coating layer can comprise polyethylene glycol (PEG). In some embodiments, the hydrophilic polymer coating layer can comprise branched PEG having at least 4 branches. In some embodiments, the low non-specific binding coating has a degree of hydrophilicity which can be measured as a water contact angle, where the water contact angle is no more than 45 degrees (e.g., no more than 5 degrees, no more than 10 degrees, no more than 15 degrees, no more than 20 degrees, no more than 25 degrees, no more than 30 degrees, no more than 35 degrees, no more than 40 degrees, or no more than 45 degrees). In some embodiments, the density of the concatemer molecules immobilized to the support or immobilized to the coating on the support is about 10²-10⁶ per mm² (e.g., about 10², 10³, 10⁴, 10⁵, or 10⁶). In some embodiments, the density of the concatemer molecules immobilized to the support or immobilized to the coating on the support is about 10⁶-10⁹ per mm² (e.g., about 10⁶, 10⁷, 10⁸, or 10⁹). In some embodiments, the density of the concatemer molecules immobilized to the support or immobilized to the coating on the support is about 10⁹-10¹² (e.g., about 10⁹, 10¹⁰, 10¹¹, or 10¹²) per mm². In some embodiments, the plurality of the concatemer molecules is immobilized to the support or immobilized to the coating on the support at pre-determined sites on the support (or the coating on the support). In some embodiments, the plurality of the concatemer molecules is immobilized to the coating on the support at random sites on the support (or the coating on the support).

In some embodiments, the distributing of step (c) can be conducted in the presence of a high-efficiency hybridization buffer which comprises: (i) a first polar aprotic solvent having a dielectric constant that is no greater than 40 (e.g., less than 10, or about 10, 15, 20, or 40) and having a polarity index of 4-9 (e.g., 4, 5, 6, 7, 8, or 9); (ii) a second polar aprotic solvent having a dielectric constant that is no greater than 115 (e.g., less than 10, 10, 15, 20, 40, 50, 75, 100, 105, 105, 110, or 115) and is present in the hybridization buffer formulation in an amount effective to denature double-stranded nucleic acids; (iii) a pH buffer system that maintains the pH of the hybridization buffer formulation in a range of about 4-8 (e.g., 4, 5, 6, 7, or 8); and (iv) a crowding agent in an amount sufficient to enhance or facilitate molecular crowding. In some embodiments, the high efficiency hybridization buffer comprises: (i) the first polar aprotic solvent comprises acetonitrile at 25-50% (e.g., 25%, 30%, 35%, 40%, 45%, or 50%) by volume of the hybridization buffer; (ii) the second polar aprotic solvent comprises formamide at 5-10% (e.g., 5%, 6%, 7%, 8%, 9%, or 10%) by volume of the hybridization buffer; (iii) the pH buffer system comprises 2-(N-morpholino)ethanesulfonic acid (MES) at a pH of 5-6.5 (e.g., about 5.0, 5.5, 6.0, or 6.5); and (iv) the crowding agent comprises polyethylene glycol (PEG) at 5-35% (e.g., 5%, 10%, 15%, 20%, 25%, 30%, or 35%) by volume of the hybridization buffer. In some embodiments, the high efficiency hybridization buffer further comprises betaine.

In some embodiments, the methods for conducting rolling circle amplification reaction on a plurality of covalently closed circular library molecules which lack hybridized first splint strands (300), wherein individual covalently closed circular library molecules (600) in the plurality comprise a first (130) or second (150) right universal adaptor sequence of a universal binding sequence for a forward amplification primer. In some embodiments, individual covalently closed circular library molecules (600) in the plurality comprise a second splint strand region (400) which includes a universal binding sequence for a third surface primer, the method comprises (a) hybridizing in solution a plurality of soluble forward amplification primers to the first or second right universal adaptor sequence which comprise the universal binding sequence for a forward amplification primer; and (b) conducting a first rolling circle amplification reaction by contacting the plurality of covalently closed circular library molecules (600) with a plurality of strand-displacing polymerases and a plurality of nucleotides (e.g., comprising bases A, G, C, T and/or U), under a condition suitable to conduct a rolling circle amplification reaction in solution using the plurality of forward amplification primers and the plurality of covalently closed circular library molecules (600) as template molecules, thereby generating a plurality of nucleic acid concatemer molecules which are still hybridized to covalently closed circular library molecules (600). In some embodiments, the methods for conducting rolling circle amplification reaction further comprises step (c): distributing the plurality of concatemer molecules onto a support having a plurality of the third surface primers immobilized thereon, under a condition suitable for hybridizing at least a portion of the concatemers to the plurality of the immobilized third surface primers thereby immobilizing the plurality of concatemer molecules. In certain embodiments, the plurality of immobilized concatemer molecules is still hybridized to covalently closed circular library molecules (600). In some embodiments, the methods for conducting rolling circle amplification reaction further comprises step (d): contacting the immobilized plurality of concatemer molecules with a plurality of strand-displacing polymerases and a plurality of nucleotides (e.g., comprising bases A, G, C, T and/or U), under a condition suitable to conduct a second rolling circle amplification reaction on the support using the plurality of covalently closed circular library molecules (600) as template molecules, thereby extending the plurality of immobilized nucleic acid concatemer molecules. In some embodiments, the first and/or the second rolling circle amplification reactions can be conducted with a plurality of nucleotides which comprise any combination of two or more of dATP, dGTP, dCTP, dTTP and/or dUTP. In some embodiments, individual immobilized concatemers are hybridized to individual third surface primers. In some embodiments, individual covalently closed circular library molecules (600) in the plurality comprise a second splint strand region (400) which also include a universal binding sequence for a fourth surface primer so that the first rolling circle amplification reaction in solution generates concatemer molecules having multiple copies of universal binding sequences for third and fourth surface primers. In some embodiments, the method comprises distributing the concatemer molecules onto a support comprising a plurality of immobilized third and fourth surface primers and incubating the concatemer molecules under a condition suitable for hybridizing at least one second splint strand region (400) of the concatemer molecules to immobilized third and fourth surface primers thereby pinning down at least one portion of the concatemer molecules to the support. In some embodiments, the immobilized concatemers can be subjected to sequencing reactions.

In some embodiments, the plurality of concatemer molecules of step (c) can be distributed onto a support comprising a plurality of the third surface primers immobilized on the support comprise the sequence 5′-GATCAGGTGAGGCTGCGACGACT-3′ (SEQ ID NO:7). In some embodiments, individual third surface primers can hybridize to a portion of a concatemer molecule having a universal binding sequence for a third surface primer, where the universal binding sequence for a third surface primer comprises the sequence 5′-AGTCGTCGCAGCCTCACCTGATC-3′ (SEQ ID NO:2, or a complementary sequence thereof).

In some embodiments, the plurality of concatemer molecules of step (c) can be distributed onto a support comprising a plurality of the fourth surface primers immobilized on the support comprise the sequence 5′-CATGTAATGCACGTACTTTCAGGGT-3′ (SEQ ID NO:1 or a complementary sequence thereof). In some embodiments, individual fourth surface primers can hybridize to a portion of the concatemer molecules having a universal binding sequence for a fourth surface primer (or a complementary sequence thereof), where the universal binding sequence for the fourth surface primer comprises the sequence 5′-CATGTAATGCACGTACTTTCAGGGT-3′ (SEQ ID NO:1).

In some embodiments, the plurality of concatemer molecules of step (c) can be distributed onto a support that is coated with one or more compounds to produce a passivated layer on the support (e.g., FIG. 8). In some embodiments, the passivated layer forms a porous or semi-porous layer. In some embodiments, the surface primer, concatemer template molecule and/or polymerase, can be attached to the passivated layer for immobilization to the support. In some embodiments, the support comprises a low non-specific binding surface that enable improved nucleic acid hybridization and amplification performance on the support. In general, the support may comprise one or more layers of a covalently or non-covalently attached low-binding, chemical modification layers, e.g., silane layers, polymer films, and one or more covalently or non-covalently attached oligonucleotides that can be used for immobilizing a plurality of nucleic acid concatemer molecules to the support. In some embodiments, the support can comprise a functionalized polymer coating layer covalently bound at least to a portion of the support via a chemical group on the support, a primer grafted to the functionalized polymer coating, and a water-soluble protective coating on the primer and the functionalized polymer coating. In some embodiments, the functionalized polymer coating comprises a poly(N-(5-azidoacetamidylpentyl)acrylamide-co-acrylamide (PAZAM). In some embodiments, the support comprises a surface coating having at least one hydrophilic polymer coating layer and at least one layer of a plurality of oligonucleotides. In some embodiments, the hydrophilic polymer coating layer can comprise polyethylene glycol (PEG). The hydrophilic polymer coating layer can comprise branched PEG having at least 4 branches (e.g., 4, 5, 6, 7, 8, 9, 10, or more branches). In some embodiments, the low non-specific binding coating has a degree of hydrophilicity which can be measured as a water contact angle, where the water contact angle is no more than 45 degrees (e.g., no more than 5 degrees, no more than degrees, no more than 15 degrees, no more than 20 degrees, no more than 25 degrees, no more than 30 degrees, no more than 35 degrees, no more than 40 degrees, or no more than 45 degrees). In some embodiments, the density of the concatemer molecules immobilized to the support or immobilized to the coating on the support is about 10²-10⁶ per mm² (e.g., about 10², 10³, 10⁴, 10⁵, or 10⁶). In some embodiments, the density of the concatemer molecules immobilized to the support or immobilized to the coating on the support is about 10⁶-10⁹ per mm² (e.g., about 10⁶, 10⁷, 10⁸, or 10⁹). In some embodiments, the density of the concatemer molecules immobilized to the support or immobilized to the coating on the support is about 10⁹-10¹² (e.g., about 10⁹, 10¹⁰, 10¹¹, or 10¹²) per mm². In some embodiments, the plurality of the concatemer molecules is immobilized to the support or immobilized to the coating on the support at pre-determined sites on the support (or the coating on the support). In some embodiments, the plurality of the concatemer molecules is immobilized to the coating on the support at random sites on the support (or the coating on the support).

In some embodiments, the distributing of step (c) can be conducted in the presence of a high-efficiency hybridization buffer which comprises: (i) a first polar aprotic solvent having a dielectric constant that is no greater than 40 (e.g., less than 10, or about 10, 15, 20, or 40) and having a polarity index of 4-9 (e.g., 4, 5, 6, 7, 8, or 9); (ii) a second polar aprotic solvent having a dielectric constant that is no greater than 115 (e.g., less than 10, 10, 15, 20, 40, 50, 75, 100, 105, 105, 110, or 115) and is present in the hybridization buffer formulation in an amount effective to denature double-stranded nucleic acids; (iii) a pH buffer system that maintains the pH of the hybridization buffer formulation in a range of about 4-8 (e.g., 4, 5, 6, 7, or 8); and (iv) a crowding agent in an amount sufficient to enhance or facilitate molecular crowding. In some embodiments, the high efficiency hybridization buffer comprises: (i) the first polar aprotic solvent comprises acetonitrile at 25-50% (e.g., 25%, 30%, 35%, 40%, 45%, or 50%) by volume of the hybridization buffer; (ii) the second polar aprotic solvent comprises formamide at 5-10% (e.g., 5%, 6%, 7%, 8%, 9%, or 10%) by volume of the hybridization buffer; (iii) the pH buffer system comprises 2-(N-morpholino)ethanesulfonic acid (MES) at a pH of 5-6.5 (e.g., about 5.0, 5.5, 6.0, or 6.5); and (iv) the crowding agent comprises polyethylene glycol (PEG) at 5-35% (e.g., 5%, 10%, 15%, 20%, 25%, 30%, or 35%) by volume of the hybridization buffer. In some embodiments, the high efficiency hybridization buffer further comprises betaine.

### In-Solution Rolling Circle Amplification Using First Splint Strands Using Circularized Library Molecules Generated Via Ds-Splint Adaptors

In some embodiments, the methods for conducting rolling circle amplification reaction on a plurality of covalently closed circular library molecules which are hybridized to first splint strands (300), wherein individual covalently closed circular library molecules (600) in the plurality comprise a second splint strand region (400) which includes a universal binding sequence for a third surface primer. In some embodiments, the method comprises (a): contacting in solution the plurality of covalently closed circular library molecules (600) which are hybridized to first splint strands (300) with a plurality of strand-displacing polymerases and a plurality of nucleotides (e.g., comprising bases A, G, C, T and/or U) under a condition suitable for conducting a first rolling circle amplification reaction using the first splint strand (300) as an amplification primer thereby generating a plurality of concatemer molecules which are still hybridized to covalently closed circular library molecules (600). See FIG. 2.

In some embodiments, the methods for conducting rolling circle amplification reaction further comprises step (b): distributing the plurality of concatemer molecules which are hybridized to a covalently closed circular library molecule (600) onto a support having a plurality of the third surface primers immobilized thereon, under a condition suitable for hybridizing at least a portion of the concatemers to the plurality of the immobilized third surface primers thereby immobilizing the plurality of concatemer molecules which are hybridized to a covalently closed circular library molecule (600).

In some embodiments, the plurality of concatemer molecules of step (b) can be distributed onto a support comprising a plurality of the third surface primers immobilized on the support comprise the sequence 5′-GATCAGGTGAGGCTGCGACGACT-3′ (SEQ ID NO:7). In some embodiments, individual third surface primers can hybridize to a portion of a concatemer molecule having a universal binding sequence for a third surface primer. In certain embodiments, the universal binding sequence for a third surface primer comprises the sequence 5′-AGTCGTCGCAGCCTCACCTGATC-3′ (SEQ ID NO:2, or a complementary sequence thereof).

In some embodiments, the plurality of concatemer molecules of step (b) can be distributed onto a support comprising a plurality of the fourth surface primers immobilized on the support comprise the sequence 5′-CATGTAATGCACGTACTTTCAGGGT-3′ (SEQ ID NO:1, or a complementary sequence thereof). In some embodiments, individual fourth surface primers can hybridize to a portion of the concatemer molecules having a universal binding sequence for a fourth surface primer (or a complementary sequence thereof). In certain embodiments, the universal binding sequence for the fourth surface primer comprises the sequence 5′-CATGTAATGCACGTACTTTCAGGGT-3′ (SEQ ID NO:1).

In some embodiments, the plurality of concatemer molecules of step (b) can be distributed onto a support that is coated with one or more compounds to produce a passivated layer on the support (e.g., FIG. 8). In some embodiments, the passivated layer forms a porous or semi-porous layer. In some embodiments, the surface primer, concatemer template molecule and/or polymerase, can be attached to the passivated layer for immobilization to the support. In some embodiments, the support comprises a low non-specific binding surface that enable improved nucleic acid hybridization and amplification performance on the support. In some embodiments, the support may comprise one or more layers of a covalently or non-covalently attached low-binding, chemical modification layers, e.g., silane layers, polymer films, and one or more covalently or non-covalently attached oligonucleotides that can be used for immobilizing a plurality of nucleic acid concatemer molecules to the support. In some embodiments, the support can comprise a functionalized polymer coating layer covalently bound at least to a portion of the support via a chemical group on the support, a primer grafted to the functionalized polymer coating, and a water-soluble protective coating on the primer and the functionalized polymer coating. In some embodiments, the functionalized polymer coating comprises a poly(N-(5-azidoacetamidylpentyl)acrylamide-co-acrylamide (PAZAM). In some embodiments, the support comprises a surface coating having at least one hydrophilic polymer coating layer and at least one layer of a plurality of oligonucleotides. In some embodiments, the hydrophilic polymer coating layer can comprise polyethylene glycol (PEG). In some embodiments, the hydrophilic polymer coating layer can comprise branched PEG having at least 4 branches (e.g., 4, 5, 6, 7, 8, 9, 10, or more branches). In some embodiments, the low non-specific binding coating has a degree of hydrophilicity which can be measured as a water contact angle, where the water contact angle is no more than 45 degrees (e.g., no more than 5 degrees, no more than 10 degrees, no more than 15 degrees, no more than 20 degrees, no more than 25 degrees, no more than 30 degrees, no more than 35 degrees, no more than 40 degrees, or no more than 45 degrees). In some embodiments, the density of the concatemer molecules immobilized to the support or immobilized to the coating on the support is about 10²-10⁶ per mm² (e.g., about 10², 10³, 10⁴, 10⁵, or 10⁶). In some embodiments, the density of the concatemer molecules immobilized to the support or immobilized to the coating on the support is about 10⁶-10⁹ per mm² (e.g., about 10⁶, 10⁷, 10⁸, or 10⁹). In some embodiments, the density of the concatemer molecules immobilized to the support or immobilized to the coating on the support is about 10⁹-10¹² (e.g., about 10⁹, 10¹⁰, 10¹¹, or 10¹²) per mm². In some embodiments, the plurality of the concatemer molecules is immobilized to the support or immobilized to the coating on the support at pre-determined sites on the support (or the coating on the support). In some embodiments, the plurality of the concatemer molecules is immobilized to the coating on the support at random sites on the support (or the coating on the support).

In some embodiments, the distributing of step (b) can be conducted in the presence of a high-efficiency hybridization buffer which comprises: (i) a first polar aprotic solvent having a dielectric constant that is no greater than 40 (e.g., less than 10, or about 10, 15, 20, or 40) and having a polarity index of 4-9 (e.g., 4, 5, 6, 7, 8, or 9); (ii) a second polar aprotic solvent having a dielectric constant that is no greater than 115 (e.g., less than 10, 10, 15, 20, 40, 50, 75, 100, 105, 105, 110, or 115) and is present in the hybridization buffer formulation in an amount effective to denature double-stranded nucleic acids; (iii) a pH buffer system that maintains the pH of the hybridization buffer formulation in a range of about 4-8 (e.g., 4, 5, 6, 7, or 8); and (iv) a crowding agent in an amount sufficient to enhance or facilitate molecular crowding. In some embodiments, the high efficiency hybridization buffer comprises: (i) the first polar aprotic solvent comprises acetonitrile at 25-50% (e.g., 25%, 30%, 35%, 40%, 45%, or 50%) by volume of the hybridization buffer; (ii) the second polar aprotic solvent comprises formamide at 5-10% (e.g., 5%, 6%, 7%, 8%, 9%, or 10%) by volume of the hybridization buffer; (iii) the pH buffer system comprises 2-(N-morpholino)ethanesulfonic acid (MES) at a pH of 5-6.5 (e.g., about 5.0, 5.5, 6.0, or 6.5); and (iv) the crowding agent comprises polyethylene glycol (PEG) at 5-35% (e.g., 5%, 10%, 15%, 20%, 25%, 30%, or 35%) by volume of the hybridization buffer. In some embodiments, the high efficiency hybridization buffer further comprises betaine.

In some embodiments, the methods for conducting rolling circle amplification reaction further comprises step (c): contacting the plurality of immobilized concatemer molecules with a plurality of strand-displacing polymerases and a plurality of nucleotides (e.g., comprising bases A, G, C, T and/or U), under a condition suitable to conduct a second rolling circle amplification reaction on the support using the plurality of covalently closed circular library molecules (600) as template molecules, thereby extending the plurality of immobilized nucleic acid concatemer molecules.

In some embodiments, the first and/or the second rolling circle amplification reactions can be conducted with a plurality of nucleotides which comprise any combination of two or more of dATP, dGTP, dCTP, dTTP and/or dUTP. In some embodiments, individual covalently closed circular library molecules (600) in the plurality comprise a second splint strand region (400) which also include a universal binding sequence for a fourth surface primer so that the first rolling circle amplification reaction in solution generates concatemer molecules having multiple copies of universal binding sequences for third and fourth surface primers. In some embodiments, the method comprises distributing the concatemer molecules onto a support comprising a plurality of immobilized third and fourth surface primers, and incubating the plurality of immobilized nucleic acid concatemer molecules under a condition suitable for hybridizing at least one second splint strand region (400) of the concatemer molecules to the immobilized third and fourth surface primers thereby pinning down at least one portion of the concatemer molecules to the support. In some embodiments, individual immobilized concatemers are hybridized to individual third surface primers. In some embodiments, the immobilized concatemers can be subjected to sequencing reactions.

### Methods for Forming a Plurality of Library-Splint Complexes Using Single-Stranded Splint Strands

In some embodiments, any of the methods described above can be used to generate nucleic acid linear library molecules each comprising sequences arranged in a 5′ to 3′ order.

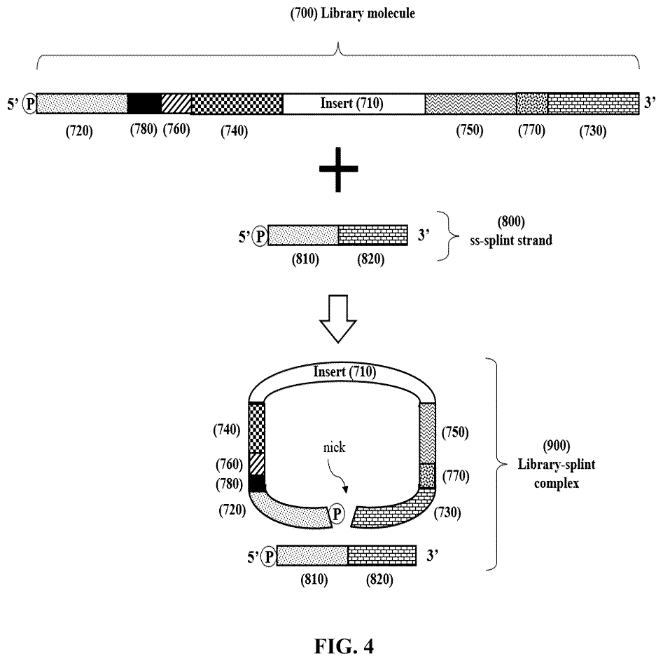

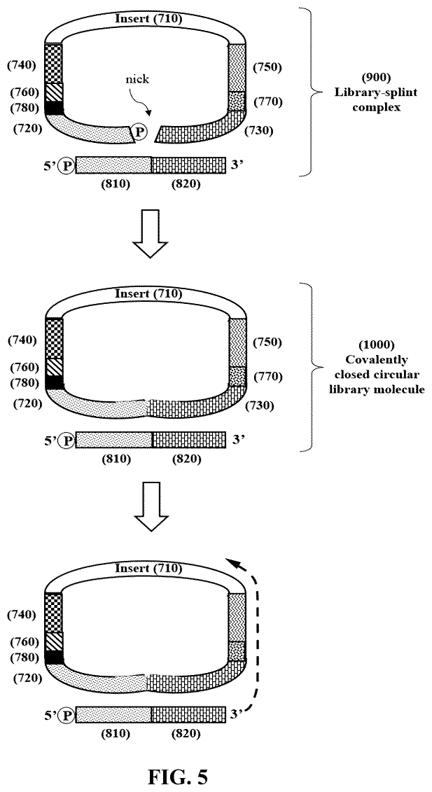

In some embodiments, a nucleic acid linear library molecules is arranged, 5′ to 3′: (i) a first left universal adaptor sequence (720) having a binding sequence for a first surface primer; (ii) a first left index sequence (760); (iii) a second left universal adaptor sequence (740) having a binding sequence for a first sequencing primer; (iv) a sequence of interest (710); (v) a second right universal adaptor sequence (750) having a binding sequence for a second sequencing primer; (vi) a first right index sequence (770); and (vii) a first right universal adaptor sequence (730) having a binding sequence for a second surface primer (e.g., see FIGS. 4-5).

In some embodiments, at least one of the linear library molecules carries at least one deaminated nucleotide base. In some embodiments, prior to circularization, the linear library molecules can be treated with a reagent that removes deaminated bases. In some embodiments, the reagent that removes deaminated bases comprises a compound that generates abasic sites at uracil bases in a nucleic acid molecule. For example, and without limitation, DNA glycosylase (UDG) can generate abasic sites at uracil bases. In some embodiments, the reagent that removes deaminated bases comprises a compound that generates a gap at abasic sites in a nucleic acid strand. For example, and without limitation, the gaps can be generated with an enzyme or a mixture of enzymes having lyase activity that breaks the phosphodiester backbone at the 5′ and 3′ sides of the abasic site to release the base-free deoxyribose and generate a gap. In some embodiments, the abasic sites can be removed using AP lyase, Endo IV endonuclease, FPG glycosylase/AP lyase, Endo VIII glycosylase/AP lyase, and combinations thereof. In some embodiments, generating the abasic sites and removal of the abasic sites to generate gaps can be achieved using a mixture of uracil DNA glycosylase and DNA glycosylase-lyase endonuclease VIII. For example, and without limitation, generating the abasic sites and removal of the abasic sites may be performed with USER™ (Uracil-Specific Excision Reagent Enzyme from New England Biolabs™) or thermolabile USER™ (also from New England Biolabs™).

In another aspect, the present disclosure provides methods for forming a plurality of library-splint complexes (900) comprising: (a) providing a plurality of single-stranded nucleic acid library molecules (700) wherein individual library molecules in the plurality include a sequence of interest (710) flanked on one side by at least a first left universal adaptor sequence (720) and flanked on the other side by at least a first right universal adaptor sequence (730) (e.g., see FIGS. 4-5).

In some embodiments, the methods for forming a plurality of library-splint complexes (900) further comprise step (b): providing a plurality of single-stranded splint strands (800) wherein individual single-stranded splint strands (800) in the plurality comprise a first region (810) that is capable of hybridizing with the at least a first left universal adaptor sequence (720) of an individual library molecule, and a second region (820) that is capable of hybridizing with the at least a first right universal adaptor sequence (730) of an individual library molecule. Exemplary single-stranded splint strands (800) are shown in FIGS. 4-5. In some embodiments, the single-stranded splint strand (800) can be 20-150 nucleotides in length, or 60-100 nucleotides in length, or 70-90 nucleotides in length, or 60-80 nucleotides in length.

In some embodiments, the methods for forming a plurality of library-splint complexes (900) further comprise step (c): hybridizing the plurality of single-stranded splint strands (800) with plurality of single-stranded nucleic acid library molecules (700). In certain embodiments, the hybridizing is conducted under a condition suitable for hybridizing individual library molecules with individual single-stranded splint strands such that the first region of one of the single-stranded splint strands (810) anneals to the at least first left universal adaptor sequence (720) of the library molecule, and such that the second region of the single-stranded splint strand (820) anneals to the at least first right universal sequence (730) of the library molecule, thereby circularizing individual library molecules to form a plurality of library-splint complexes (900). In some embodiments, the library-splint complex (900) comprises a nick between the terminal 5′ and 3′ ends of the library molecule (e.g., FIGS. 4-5). In some embodiments, the nick is enzymatically ligatable.

In some embodiments, in the methods for forming a plurality of library-splint complexes (900), the first region of the single-stranded splint strand (810) comprises a first universal adaptor sequence which can hybridize to a first universal binding sequence at one end of a linear nucleic acid library molecule (720). In some embodiments, the first region of the single-stranded splint strand (810) includes a first universal adaptor sequence which comprises a universal binding sequence for a forward or reverse sequencing primer, a universal binding sequence for a first or second surface primer, a universal binding sequence for a forward or reverse amplification primer, or a universal binding sequence for a compaction oligonucleotide.

In some embodiments, in the methods for forming a plurality of library-splint complexes (900), the second region of the single-stranded splint strand (820) comprises a second universal adaptor sequence which can hybridize to a second universal binding sequence at the other end of the linear nucleic acid library molecule. In some embodiments, the second region of the single-stranded splint strand (820) includes a second universal adaptor sequence which comprises a universal binding sequence for a forward or reverse sequencing primer, a universal binding sequence for a first or second surface primer, a universal binding sequence for a forward or reverse amplification primer, or a universal binding sequence for a compaction oligonucleotide.

In some embodiments, in the methods for forming a plurality of library-splint complexes (900), the single-stranded splint strands (800) comprise one or more phosphorothioate linkage at their 5′ and/or 3′ ends to confer exonuclease resistance. In some embodiments, the single-stranded splint strands (800) comprise one or more phosphorothioate linkage at an internal position to confer endonuclease resistance. In some embodiments, the single-stranded splint strands (800) comprise one or more 2′-O-methylcytosine bases at their 5′ and/or 3′ ends, or at an internal position. In some embodiments, the 5′ end of the single-stranded splint strand (800) is phosphorylated or non-phosphorylated. In some embodiments, the 3′ end of the single-stranded splint strand (800) comprises a terminal 3′ OH group or a terminal 3′ blocking group.

In some embodiment, in the methods for forming a plurality of library-splint complexes (900), the first region of the single-stranded splint strand (810) can hybridize to a sense or anti-sense strand of a double-stranded nucleic acid library molecule. In some embodiments, in the library-splint complex (900), the second region of the single-stranded splint strand (820) can hybridize to a sense or anti-sense strand of a double-stranded nucleic acid library molecule. In some embodiments, the double-stranded nucleic acid library molecule can be denatured to generate the single-stranded sense and anti-sense library strands. In certain embodiments, the double-stranded nucleic acid library molecule can be denatured to generate the single-stranded nucleic acid library molecules (700) of step (a).

In some embodiments, in the methods for forming a plurality of library-splint complexes (900), the first and second regions (210 and 220, respectively) of the single-stranded splint strand do not hybridize to the sequence of interest (710).

In some embodiments, in the methods for forming a plurality of library-splint complexes (900), the nucleic acid library molecule (700) further comprises a second left universal adaptor sequence (740). In some embodiments, the nucleic acid library molecule (700) further comprises a second right universal adaptor sequence (750). In some embodiments, the nucleic acid library molecule (700) can further comprise additional left and/or right universal adaptor sequences.

In some embodiments, in the methods for forming a plurality of library-splint complexes (900), the nucleic acid library molecule (700) further comprises a first left index sequence (760) and/or a first right index sequence (770). In some embodiments, the first left index sequence (760) comprises a sample index sequence. The first left index sequence (760) can be 3-20 (e.g., 3, 4, 5, 6, 7, 8, 9, 10, 11, 12, 13, 14, 15, 16, 17, 18, 19, or 20) nucleotides in length. In some embodiments, the first right index sequence (770) comprises another sample index sequence. In some embodiments, the first right index sequence (770) can be 3-20 (e.g., 3, 4, 5, 6, 7, 8, 9, 10, 11, 12, 13, 14, 15, 16, 17, 18, 19, or 20) nucleotides in length. In some embodiments, the sequences of the left and right sample index sequences (e.g., (760) and (770)) can be the same or different from each other. In some embodiments, the sample index sequences can be used to distinguish sequences of interest obtained from different sample sources in a multiplex assay. A list of exemplary first left index sequences (760) and first right index sequences (770) is provided in Table 2 at FIG. 7. In some embodiments, the first left index sequence (760) can include a short random sequence (e.g., NNN) or lack a short random sequence. In some embodiments, the first right index sequence (770) can include a short random sequence (e.g., NNN) or lack a short random sequence. In some embodiments, the short random sequence (e.g., NNN) can be 3-20 (e.g., 3, 4, 5, 6, 7, 8, 9, 10, 11, 12, 13, 14, 15, 16, 17, 18, 19, or 20) nucleotides in length.

In some embodiments, Multiplex workflows are enabled by preparing sample-indexed libraries using one or both index sequences (e.g., left and/or right index sequences). In some embodiments, the first left index sequences (760) and/or first right index sequences (770) can be employed to prepare separate sample-indexed libraries using input nucleic acids isolated from different sources. In some embodiments, the sample-indexed libraries can be pooled together to generate a multiplex library mixture, and the pooled libraries can be circularized, amplified and/or sequenced. In some embodiments, the sequences of the insert region along with the first left index sequence (760) and/or first right index sequence (770) can be used to identify the source of the input nucleic acids. In some embodiments, any number of sample-indexed libraries can be pooled together, for example 2-10 (e.g., about 2, 3, 4, 5, 6, 7, 8, 9, or libraries. In some embodiments, 10-50 (e.g., about 10, 20, 30, 40, or 50) sample-indexed libraries are pooled together. In some embodiments, 50-100 (e.g., about 50, 60, 70, 80, 90, or 100) sample-indexed libraries are pooled together. In some embodiments, 100-200 (e.g., about 100, 110, 120, 130, 140, 150, 160, 170, 180, 190, or 200) sample-indexed libraries are pooled together. Exemplary nucleic acid sources include naturally occurring, recombinant, or chemically synthesized sources. Exemplary nucleic acid sources include, without limitation, single cells, a plurality of cells, tissue, biological fluid, environmental sample, or whole organism. Exemplary nucleic acid sources include, without limitation, fresh, frozen, fresh-frozen, or archived sources (e.g., formalin-fixed paraffin-embedded; FFPE). The skilled artisan will recognize that the nucleic acids can be isolated from many other sources. In some embodiments, the nucleic acid library molecules can be prepared in single-stranded or double-stranded form.

In some embodiments, in the methods for forming a plurality of library-splint complexes (900), the nucleic acid library molecule (700) further comprises: an optional first left unique identification sequence (780) and/or an optional first right unique identification sequence (790). In some embodiments, the first left unique identification sequence (780) and the first right unique identification sequence (790) each comprise a sequence that is used to uniquely identify an individual sequence of interest (e.g., insert sequence) to which the unique adaptors are appended in a population of other sequence of interest molecules. In some embodiments, the first left unique identification sequence (780) and/or the first right unique identification sequence (790) can be used for molecular tagging. In some embodiments, the unique identification sequence comprises 2-12 or more (e.g., about 2, 3, 4, 5, 6, 7, 8, 9, 10, 11, 12, or more than 12) nucleotides having a known sequence. For example, and without limitation, the unique identification sequence comprises a known random sequence where a nucleotide at each position is randomly selected from nucleotides having a base A, G, C, T or U. In some embodiments, the unique identification sequences (780) and/or (790) can be used for molecular tagging procedures.

In some embodiments, in the methods for forming a plurality of library-splint complexes (900), the nucleic acid library molecule (700) comprises any one or any combination and in any order of two or more: a first left universal adaptor sequence (720); a second left universal adaptor sequence (740); a first left index sequence (760); a first left unique identification sequence (780); a first right universal adaptor sequence (730); a second right universal adaptor sequence (750); a first right index sequence (770); and/or a first right unique identification sequence (790).

In some embodiments, in the methods for forming a plurality of library-splint complexes (900), the first left universal adaptor sequence (720) and/or the second left universal adaptor sequence (740), comprises: a universal binding sequence for a forward or reverse sequencing primer; a universal binding sequence for a first or second surface primer; a universal binding sequence for a forward or reverse amplification primer; and/or a universal binding sequence for a compaction oligonucleotide. In some embodiments, the nucleic acid library molecule (700) can further comprise additional left universal adaptor sequences.

In some embodiments, in the methods for forming a plurality of library-splint complexes (900), the first right universal adaptor sequence (730) and/or the second right universal adaptor sequence (750), comprises: a universal binding sequence for a forward or reverse sequencing primer; a universal binding sequence for a first or second surface primer; a universal binding sequence for a forward or reverse amplification primer; and/or a universal binding sequence for a compaction oligonucleotide. In some embodiments, the nucleic acid library molecule (700) can further comprise additional right universal adaptor sequences.

In another aspect, the present disclosure provides methods for forming a plurality of library-splint complexes (900) comprising: (a) providing a plurality of single-stranded splint strands (800) wherein individual single-stranded splint strands (800) comprises regions arranged in a 5′ to 3′ order (i) a first region (810) having a universal binding sequence that hybridizes with a sequence on one end of the linear single stranded library molecule (e.g., 120), and (ii) a second region (820) having a universal binding sequence that hybridizes with a sequence on the other end of the linear single stranded library molecule (e.g., 130). In some embodiments, the methods for forming a plurality of library-splint complexes (900) further comprises step (b): hybridizing the plurality of single-stranded splint strands (800) with a plurality of single-stranded nucleic acid library molecules (700) wherein individual library molecules comprise regions arranged in a 5′ to 3′ order: (i) a first left universal adaptor sequence (720) having a binding sequence for a first surface primer; (ii) a second left universal adaptor sequence (740) having a binding sequence for a first or second sequencing primer; (iii) a sequence of interest (710); (iv) a second right universal adaptor sequence (750) having a binding sequence for a second or first sequencing primer; and (v) a first right universal adaptor sequence (730) having a binding sequence for a second surface primer. In certain embodiments, the hybridizing is conducted under a condition suitable to hybridize the single-stranded splint strand (800) to the library molecule (700) thereby circularizing the library molecule to generate a library-splint complex (900), such that the first region (810) of the single-stranded splint strand is hybridized to the binding sequence for the first surface primer (720), and such that the second region (820) of the single-stranded splint strand is hybridized to the binding sequence for the second surface primer (730). In certain embodiments, the library-splint complex (900) comprises a nick between the terminal 5′ and 3′ ends of the library molecule. In certain embodiments, the nick is enzymatically ligatable (e.g., see FIG. 4). In some embodiments, the plurality of single-stranded nucleic acid library molecules (700) further comprises a first left index sequence (760) and/or a first right index sequence (770) (e.g., see FIG. 4). In some embodiments, in a given library-splint complex (900) of the plurality, the sequences of the first left index (760) and the first right index (770) are the same or different from each other. In some embodiments, the first left index sequence (760) can be 3-20 (e.g., about 3, 4, 5, 6, 7, 8, 9, 10, 11, 12, 13, 14, 15, 16, 17, 18, 19, or 20 bases) nucleotides in length. The first right index sequence (770) can be 3-20 (e.g., about 3, 4, 5, 6, 7, 8, 9, 10, 11, 12, 13, 14, 15, 16, 17, 18, 19, or 20 bases) nucleotides in length. In some embodiments, the first left index sequence (760) and/or the first right index sequence (770) can include a short random sequence (e.g., NNN). In some embodiments, the short random sequence can be 3-20 (e.g., about 3, 4, 5, 6, 7, 8, 9, 11, 12, 13, 14, 15, 16, 17, 18, 19, or 20 bases) nucleotides in length. A list of exemplary first left index sequences (760) and first right index sequences (770) is provided in Tables 1-2 at FIG. 7. In some embodiments, the plurality of single-stranded nucleic acid library molecules (700) further comprises a first left unique identification sequence (780) and/or a first right unique identification sequence (790) which can be used for molecular tagging (e.g., see FIG. 4).

In some embodiments, in the methods, the first left universal adaptor sequence (720) in the library molecules comprise the sequence 5′-CATGTAATGCACGTACTTTCAGGGT-3′ (SEQ ID NO:1).

In some embodiments, in the methods, the first left universal adaptor sequence (720) in the library molecules comprise the sequence 5′-AATGATACGGCGACCACCGA—3′ (SEQ ID NO:204).

In some embodiments, in the methods, the second left universal adaptor sequence (740) in the library molecules comprise the sequence 5′-CGTGCTGGATTGGCTCACCAGACACCTTCCGACAT-3′ (SEQ ID NO:209).

In some embodiments, in the methods, the second left universal adaptor sequence (740) in the library molecules comprise the sequence 5′-ACACTCTTTCCCTACACGACGCTCTTCCGATCT-3′ (SEQ ID NO:207).

In some embodiments, in the methods, the second left universal adaptor sequence (740) in the library molecules comprise the sequence 5′-TCGTCGGCAGCGTCAGATGTGTATAAGAGACAG-3′ (SEQ ID NO:208).

In some embodiments, in the methods, the second right universal adaptor sequence (750) in the library molecules comprise the sequence ATGTCGGAAGGTGTGCAGGCTACCGCTTGTCAACT-3′ (SEQ ID NO:212).

In some embodiments, in the methods, the second right universal adaptor sequence (750) in the library molecules comprise the sequence AGATCGGAAGAGCACACGTCTGAACTCCAGTCAC-3′ (SEQ ID NO:210).

In some embodiments, in the methods, the second right universal adaptor sequence (750) in the library molecules comprise the sequence CTGTCTCTTATACACATCTCCGAGCCCACGAGAC-3′ (SEQ ID NO:211).

In some embodiments, in the methods, the first right universal adaptor sequence (730) in the library molecules comprise the sequence 5′-AGTCGTCGCAGCCTCACCTGATC-3′ (SEQ ID NO:2).

In some embodiments, in the methods, the first right universal adaptor sequence (730) in the library molecules comprise the sequence 5′-TCGTATGCCGTCTTCTGCTTG-3′ (SEQ ID NO:213).

In some embodiments, in the methods, the first region of the single-stranded splint strand (810) includes a universal binding sequence for a first left universal adaptor sequence (720) of a library molecule, where the first region (810) comprises the sequence ACCCTGAAAGTACGTGCATTACATG-3′ (SEQ ID NO:6).

In some embodiments, in the methods, the second region of the single-stranded splint strand (820) includes a universal binding sequence for a first right universal adaptor sequence (730) of a library molecule, where the second region (820) comprises the sequence GATCAGGTGAGGCTGCGACGACT-3′ (SEQ ID NO:7).

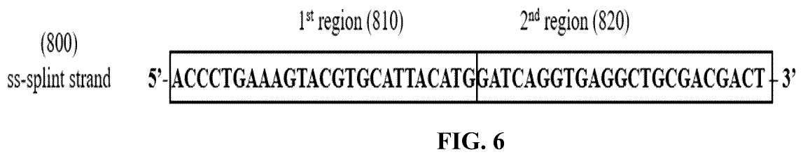

In some embodiments, in the methods, the single-stranded splint strand (800) comprises the sequence ACCCTGAAAGTACGTGCATTACATGGATCAGGTGAGGCTGCGACGACT-3′ (SEQ ID NO:8). For example, see FIG. 6.

In some embodiments, in the methods, the first region of the single-stranded splint strand (810) includes a universal binding sequence for a first left universal adaptor sequence (720) of a library molecule, where the first region (810) comprises the sequence TCGGTGGTCGCCGTATCATT-3′ (SEQ ID NO:4).

In some embodiments, in the methods, the second region of the single-stranded splint strand (820) includes a universal binding sequence for a first right universal adaptor sequence (730) of a library molecule, where the second region (820) comprises the sequence CAAGCAGAAGACGGCATACGA-3′ (SEQ ID NO:5).

In some embodiments, in the methods, the single-stranded splint strand (800) comprises the sequence TCGGTGGTCGCCGTATCATTCAAGCAGAAGACGGCATACGA-3′ (SEQ ID NO:214).

In some embodiments, any of the methods for forming a plurality of library-splint complexes (900) described herein can further comprise at least one enzymatic reaction, including a phosphorylation reaction, ligation reaction and/or exonuclease reaction. In some embodiments, the enzymatic reactions can be conducted sequentially. In some embodiments, the enzymatic reactions can be conducted essentially simultaneously. In some embodiments, the enzymatic reactions can be conducted in a single reaction vessel. Alternatively, in some embodiments, a first enzymatic reaction can be conducted in a first reaction vessel, then transferred to a second reaction vessel where the second enzymatic reaction is conducted, then transferred to a third reaction vessel where the third enzymatic reaction is conducted, etc.

In some embodiments, any of the methods for forming a plurality of library-splint complexes (900) described herein further comprise conducting separate and sequential phosphorylation and ligation reactions which are conducted in separate reaction vessels. In some embodiments, the methods for forming a plurality of library-splint complexes (900) further comprise step (el): contacting in a first reaction vessel the plurality of the single-stranded splint strands (800) and the plurality of the single-stranded nucleic acid library molecules (700) with a T4 polynucleotide kinase enzyme under a condition suitable to phosphorylate the 5′ ends of the plurality of single-stranded splint strands (800) and/or the plurality of single-stranded nucleic acid library molecules (700); and transferring the phosphorylation reaction to a second reaction vessel. In some embodiments, the methods for forming a plurality of library-splint complexes (900) further comprise step (d1): contacting in the second reaction vessel the plurality of phosphorylated single-stranded splint strands (800) and the plurality of phosphorylated single-stranded nucleic acid library molecules (700) with a ligase, under a condition suitable to enzymatically ligate the nicks, thereby generating a plurality of covalently closed circular library molecules (1000) each hybridized to a single-stranded splint strand (800). In some embodiments, the ligase enzyme comprises T7 DNA ligase, T3 ligase, T4 ligase, or Taq ligase.

In some embodiments, any of the methods for forming a plurality of library-splint complexes (900) described herein further comprise conducting sequential phosphorylation and ligation reactions which are conducted sequentially in the same reaction vessel. In some embodiments, the methods for forming a plurality of library-splint complexes (900) further comprise step (c2): contacting in a first reaction vessel the plurality of the single-stranded splint strands (800) and the plurality of the single-stranded nucleic acid library molecules (700) with a T4 polynucleotide kinase enzyme under a condition suitable to phosphorylate the 5′ ends of the plurality of single-stranded splint strands (800) and the plurality of single-stranded nucleic acid library molecules (700). In some embodiments, the methods for forming a plurality of library-splint complexes (900) further comprise step (d2): contacting in the same first reaction vessel the phosphorylated single-stranded splint strands (800) and the phosphorylated single-stranded nucleic acid library molecules (700) with a ligase under a condition suitable to enzymatically ligate the nicks, thereby generating a plurality of covalently closed circular library molecules (1000) each hybridized to a single-stranded splint strand (800). In some embodiments, the ligase enzyme comprises T7 DNA ligase, T3 ligase, T4 ligase, or Taq ligase.

In some embodiments, any of the methods for forming a plurality of library-splint complexes (900) described herein further comprise conducting essentially simultaneous phosphorylation and ligation reactions which are conducted together in the same reaction vessel. In some embodiments, the methods for forming a plurality of library-splint complexes (900) further comprise step (c3): contacting in a first reaction vessel the plurality of the single-stranded splint strands (800) and the plurality of the single-stranded nucleic acid library molecules (700) with a (i) T4 polynucleotide kinase enzyme and (ii) a ligase enzyme, under a condition suitable to phosphorylate the 5′ ends of the plurality of single-stranded splint strands (800) and the plurality of single-stranded nucleic acid library molecules (700), and the conditions are suitable to enzymatically ligate the nicks, thereby generating a plurality of covalently closed circular library molecules (1000) each hybridized to a single-stranded splint strand (800). In some embodiments, the ligase enzyme comprises T7 DNA ligase, T3 ligase, T4 ligase, or Taq ligase.

In some embodiments, any of the methods for forming a plurality of library-splint complexes (900) described herein further comprise the optional step of enzymatically removing the plurality of single-stranded splint strands (800) from the plurality of covalently closed circular library molecules (1000), which comprises the step: contacting the plurality of covalently closed circular library molecules (1000) with at least one exonuclease enzyme to remove the plurality of single-stranded splint strands (800) and retaining the plurality of covalently closed circular library molecules (1000). In some embodiments, the exonuclease reaction can be conducted in the same reaction buffer used to conduct the phosphorylation and/or ligation reactions, or in a different reaction buffer. In some embodiments, the exonuclease reaction can be conducted in a third reaction vessel after conducting the phosphorylation reaction in the first reaction vessel (step c1, see above), and conducting the ligation reaction in the second reaction vessel (step d1, see above). In some embodiments, the exonuclease reaction can be conducted in the first reaction vessel after conducting the phosphorylation reaction in the first reaction vessel (step c2, see above), and conducting the sequential ligation reaction in the first reaction vessel (step d2, see above). In some embodiments, the exonuclease reaction can be conducted in the first reaction vessel after conducting the essentially simultaneous phosphorylation and ligation reactions in the first reaction vessel (step c3, see above). In some embodiments, the at least one exonuclease enzyme comprises any combination of two or more of exonuclease I, thermolabile exonuclease I and/or T7 exonuclease.

In some embodiments, covalently closed circular library molecules (1000) can be generated using any of the methods described above. In some embodiments, individual covalently closed circular library molecules comprise a sequence of interest operably linked on both sides by at least one nucleic acid adaptor sequence. In some embodiments, at least one covalently closed circular library molecule carries at least one deaminated nucleotide base. In some embodiments, the covalently closed circular library molecules can be treated with a reagent that removes deaminated bases. In some embodiments, the reagent that removes deaminated bases comprises a compound that generates abasic sites at uracil bases in a nucleic acid molecule. For example, and without limitation, DNA glycosylase (UDG) can generate abasic sites at uracil bases. In some embodiments, the reagent that removes deaminated bases comprises a compound that generates a gap at abasic sites in a nucleic acid strand. For example, and without limitation, the gaps can be generated with an enzyme or a mixture of enzymes having lyase activity that breaks the phosphodiester backbone at the 5′ and 3′ sides of the abasic site to release the base-free deoxyribose and generate a gap. In some embodiments, the abasic sites can be removed using AP lyase, Endo IV endonuclease, FPG glycosylase/AP lyase, Endo VIII glycosylase/AP lyase, and combinations thereof. In some embodiments, generating the abasic sites and removal of the abasic sites to generate gaps can be achieved using a mixture of uracil DNA glycosylase and DNA glycosylase-lyase endonuclease VIII. For example, and without limitation, generating the abasic sites and removal of the abasic sites to generate gaps can be achieved using USER™ (Uracil-Specific Excision Reagent Enzyme from New England Biolabs™) or thermolabile USER™ (also from New England Biolabs™)

### Methods for Rolling Circle Amplification Using Circularized Library Molecules Generated Via Ss-Splint Strands

In another aspect, the present disclosure provides methods for conducting rolling circle amplification reaction on the covalently closed circular library molecules (1000). In some embodiments, the rolling circle amplification reaction can be conducted after the phosphorylation and ligation reactions, or after the ligation reaction. In some embodiments, the rolling circle amplification reaction can be conducted on covalently closed circular library molecules (1000) that are hybridized to the single-stranded splint strands (800). In some embodiments, the rolling circle amplification reaction can be conducted on covalently closed circular library molecules (1000) that are no longer hybridized to the single-stranded splint strands (800), for example following the exonuclease reaction. In some embodiments, the covalently closed circular library molecules (1000) can be distributed onto a support and then be subjected to rolling circle amplification reaction. In some embodiments, the covalently closed circular library molecules (1000) can be subjected to rolling circle amplification reaction in-solution and then distributed onto a support. In some embodiments, the rolling circle amplification reactions can employ the retained single-stranded splint strand (800) as an amplification primer, or the single-stranded splint strand (800) can be removed (e.g., via exonuclease digestion) and replaced with a soluble amplification primer.

### On-Support Rolling Circle Amplification Using Circularized Library Molecules Generated Via Ss-Splint Strands

In some embodiments, the methods for conducting rolling circle amplification reaction on a plurality of covalently closed circular library molecules which lack hybridized single-stranded splint strands (800). In some embodiments, individual covalently closed circular library molecules (1000) in the plurality comprise a universal binding sequence for a first surface primer, comprise step (a): distributing the plurality of covalently closed circular library molecules (1000) onto a support having a plurality of the first surface primers immobilized on the support, under a condition suitable for hybridizing individual covalently closed circular library molecules (1000) to individual immobilized first surface primers, thereby immobilizing the plurality of covalently closed circular library molecules (1000) to the support.

In some embodiments, in the methods for conducting rolling circle amplification reaction, the plurality of the first surface primers immobilized on the support comprise the sequence 5′-GATCAGGTGAGGCTGCGACGACT-3′ (SEQ ID NO:7).

In some embodiments, in the methods for conducting rolling circle amplification reaction, the plurality of the first surface primers immobilized on the support comprise the sequence 5′-CAAGCAGAAGACGGCATACGA-3′ (SEQ ID NO:5).

In some embodiments, individual first surface primers can hybridize to a covalently closed circular library molecule (1000) having a universal binding sequence for the first surface primer.

In some embodiments, the methods for conducting rolling circle amplification reaction further comprises step (b): contacting the plurality of immobilized covalently closed circular library molecules (1000) with a plurality of strand-displacing polymerases and a plurality of nucleotides, under a condition suitable to conduct a rolling circle amplification reaction on the support using the plurality of first surface primers as immobilized amplification primers and the plurality of covalently closed circular library molecules (1000) as template molecules, thereby generating a plurality of nucleic acid concatemer molecules immobilized to the first surface primers. In some embodiments, the plurality of nucleotides comprises any combination of two or more of dATP, dGTP, dCTP, dTTP, and/or dUTP. In some embodiments, individual immobilized concatemers are covalently joined to individual first surface primers. In some embodiments, individual covalently closed circular library molecules (1000) in the plurality comprise universal binding sequences for a first and second surface primer (e.g., (720) and (730) respectively) so that the rolling circle amplification reaction generates concatemer molecules having multiple tandem copies of universal binding sequences for first and second surface primers. In some embodiments, the support further comprises a plurality of second surface primers. In some embodiments, the immobilized second surface primers serve to pin down at least one portion of the concatemer molecules to the support. In some embodiments, the immobilized second surface primers have a non-extendible 3′ end and cannot be used for amplification. In some embodiments, the immobilized concatemers can be subjected to sequencing reactions.

In some embodiments, the plurality of the second surface primers immobilized on the support comprise the sequence 5′-CATGTAATGCACGTACTTTCAGGGT-3′ (SEQ ID NO:1 or a complementary sequence thereof).

In some embodiments, the plurality of the second surface primers immobilized on the support comprise the sequence 5′-AATGATACGGCGACCACCGA-3′ (SEQ ID NO:204 or a complementary sequence thereof).

In some embodiments, individual second surface primers can hybridize to a portion of the concatemer molecules having a universal binding sequence for the second surface primer. In some embodiments the immobilized second surface primers serve to pin down at least one portion of the concatemer molecules to the support. In some embodiments, the immobilized second surface primers have a non-extendible 3′ end and cannot be used for amplification. In some embodiments, the immobilized concatemers can be subjected to sequencing reactions.

In some embodiments, in the methods for conducting rolling circle amplification reaction, the plurality of covalently closed circular library molecules (1000) can be distributed onto a support that is coated with one or more compounds to produce a passivated layer on the support (e.g., FIG. 8). In some embodiments, the passivated layer forms a porous or semi-porous layer. In some embodiments, one or more types of surface primers, concatemer template molecules and/or polymerases, can be attached to the passivated layer for immobilization to the support. In some embodiments, the support comprises a low non-specific binding surface that enable improved nucleic acid hybridization and amplification performance on the support. In some embodiments, the support may comprise one or more layers of a covalently or non-covalently attached low-binding, chemical modification layers, e.g., silane layers, polymer films, and one or more covalently or non-covalently attached oligonucleotides that can be used for immobilizing a plurality of nucleic acid concatemer molecules to the support. In some embodiments, the support can comprise a functionalized polymer coating layer covalently bound at least to a portion of the support via a chemical group on the support, a primer grafted to the functionalized polymer coating, and a water-soluble protective coating on the primer and the functionalized polymer coating. In some embodiments, the functionalized polymer coating comprises a poly(N-(5-azidoacetamidylpentyl)acrylamide-co-acrylamide (PAZAM). In some embodiments, the support comprises a surface coating having at least one hydrophilic polymer coating layer and at least one layer of a plurality of oligonucleotides. In some embodiments, the hydrophilic polymer coating layer can comprise polyethylene glycol (PEG). In some embodiments, the hydrophilic polymer coating layer can comprise branched PEG having at least 4 branches (e.g., 4, 5, 6, 7, 8, 9, 10, or more branches). In some embodiments, the low non-specific binding coating has a degree of hydrophilicity which can be measured as a water contact angle, where the water contact angle is no more than 45 degrees (e.g., no more than 5 degrees, no more than 10 degrees, no more than 15 degrees, no more than 20 degrees, no more than 25 degrees, no more than 30 degrees, no more than 35 degrees, no more than 40 degrees, or no more than 45 degrees). In some embodiments, the density of the covalently closed circular library molecules (1000) immobilized to the support or immobilized to the coating on the support is about 10²-10⁶ (e.g., about 10², 10³, 10⁴, 10⁵, or 10⁶). In some embodiments, the density of the covalently closed circular library molecules (600) immobilized to the support or immobilized to the coating on the support is about 10⁶-10⁹ per mm² (e.g., about 10⁶, 10⁷, 10⁸, or 10⁹). In some embodiments, the density of the covalently closed circular library molecules (600) immobilized to the support or immobilized to the coating on the support is about 10⁹-10¹² (e.g., about 10⁹, 10¹⁰, 10¹¹, or 10¹²) per mm². In some embodiments, the plurality of covalently closed circular library molecules (1000) is immobilized to the support or immobilized to the coating on the support at pre-determined sites on the support (or the coating on the support). In some embodiments, the plurality of covalently closed circular library molecules (600) is immobilized to the support or immobilized to the coating on the support at random sites on the support (or the coating on the support).

In some embodiments, in the methods for conducting rolling circle amplification reaction, the distributing of step (a) (e.g., distributing the plurality of covalently closed circular library molecules (1000) onto a support) can be conducted in the presence of a high-efficiency hybridization buffer which comprises: (i) a first polar aprotic solvent having a dielectric constant that is no greater than 40 and having a polarity index of 4-9; (ii) a second polar aprotic solvent having a dielectric constant that is no greater than 115 and is present in the hybridization buffer formulation in an amount effective to denature double-stranded nucleic acids; (iii) a pH buffer system that maintains the pH of the hybridization buffer formulation in a range of about 4-8; and (iv) a crowding agent in an amount sufficient to enhance or facilitate molecular crowding. In some embodiments, the high efficiency hybridization buffer comprises: (i) the first polar aprotic solvent comprises acetonitrile at 25-50% by volume of the hybridization buffer; (ii) the second polar aprotic solvent comprises formamide at 5-10% by volume of the hybridization buffer; (iii) the pH buffer system comprises 2-(N-morpholino)ethanesulfonic acid (MES) at a pH of 5-6.5; and (iv) the crowding agent comprises polyethylene glycol (PEG) at 5-35% by volume of the hybridization buffer. In some embodiments, the high efficiency hybridization buffer further comprises betaine.

### In-Solution Rolling Circle Amplification Using Soluble Amplification Primers Using Circularized Library Molecules Generated Via Ss-Splint Strands

In some embodiments, the methods for conducting rolling circle amplification reaction on a plurality of covalently closed circular library molecules (1000) which lack hybridized single-stranded splint strands (800), wherein individual covalently closed circular library molecules (1000) in the plurality comprise a universal binding sequence for a forward amplification primer and a universal binding sequence for a first surface primer. In some embodiments, the method comprises: (a) hybridizing in solution a plurality of covalently closed circular library molecules and a plurality of soluble forward amplification primers; and (b) conducting a first rolling circle amplification reaction by contacting the plurality of covalently closed circular library molecules (1000) with a plurality of strand-displacing polymerases and a plurality of nucleotides, under a condition suitable to conduct a rolling circle amplification reaction in solution using the plurality of forward amplification primers and the plurality of covalently closed circular library molecules (1000) as template molecules, thereby generating a plurality of nucleic acid concatemer molecules having a portion which are still hybridized to their covalently closed circular library molecules (1000). In some embodiments, the methods for conducting rolling circle amplification reaction further comprises step (c): distributing the plurality of concatemer molecules onto a support having a plurality of the first surface primers immobilized thereon, under a condition suitable for hybridizing at least a portion of the concatemers to the plurality of the immobilized first surface primers thereby immobilizing the plurality of concatemer molecules. In some embodiments, the plurality of immobilized concatemer molecules is still hybridized to their covalently closed circular library molecules (1000). In some embodiments, the methods for conducting rolling circle amplification reaction further comprises step (d): contacting the immobilized plurality of concatemer molecules with a plurality of strand-displacing polymerases and a plurality of nucleotides, under a condition suitable to conduct a second rolling circle amplification reaction on the support using the plurality of covalently closed circular library molecules (1000) as template molecules, thereby extending the plurality of immobilized nucleic acid concatemer molecules. In some embodiments, the first and/or the second rolling circle amplification reactions can be conducted with a plurality of nucleotides which comprise any combination of two or more of dATP, dGTP, dCTP, dTTP and/or dUTP. In some embodiments, individual immobilized concatemers are hybridized to individual first surface primers. In some embodiments, individual covalently closed circular library molecules (1000) in the plurality comprise universal binding sequences for a first and second surface primer (e.g., (720) and (730) respectively) so that the in-solution rolling circle amplification reaction generates concatemer molecules having multiple tandem copies of universal binding sequences for first and second surface primers. In some embodiments, the support further comprises a plurality of second surface primers. In some embodiments, the immobilized second surface primers serve to pin down at least one portion of the concatemer molecules to the support. In some embodiments, the immobilized second surface primers have a non-extendible 3′ end and cannot be used for amplification. In some embodiments, the immobilized concatemers can be subjected to sequencing reactions.

In some embodiments, in the methods for conducting rolling circle amplification reaction, the plurality of the first surface primers immobilized on the support comprise the sequence 5′-GATCAGGTGAGGCTGCGACGACT-3′ (SEQ ID NO:7). In some embodiments, individual first surface primers can hybridize to a covalently closed circular library molecule (1000) having a universal binding sequence for the first surface primer.

In some embodiments, in the methods for conducting rolling circle amplification reaction, the plurality of the first surface primers immobilized on the support comprise the sequence 5′-CAAGCAGAAGACGGCATACGA-3′ (SEQ ID NO:5). In some embodiments, individual first surface primers can hybridize to a covalently closed circular library molecule (1000) having a universal binding sequence for the first surface primer.

In some embodiments, the plurality of the second surface primers immobilized on the support comprise the sequence 5′-CATGTAATGCACGTACTTTCAGGGT-3′ (SEQ ID NO:1 or a complementary sequence thereof). In some embodiments, individual second surface primers can hybridize to a portion of the concatemer molecules having a universal binding sequence for the second surface primer.

In some embodiments, the plurality of the second surface primers immobilized on the support comprise the sequence 5′-AATGATACGGCGACCACCGA-3′ (SEQ ID NO:204 or a complementary sequence thereof). In some embodiments, individual second surface primers can hybridize to a portion of the concatemer molecules having a universal binding sequence for the second surface primer.

In some embodiments the immobilized second surface primers serve to pin down at least one portion of the concatemer molecules to the support. In some embodiments, the immobilized second surface primers have a non-extendible 3′ end and cannot be used for amplification. In some embodiments, the immobilized concatemers can be subjected to sequencing reactions.

In some embodiments, the plurality of concatemer molecules of step (c) can be distributed onto a support that is coated with one or more compounds to produce a passivated layer on the support (e.g., FIG. 8). In some embodiments, the passivated layer forms a porous or semi-porous layer. In some embodiments, the one or more types of surface primers, concatemer template molecules and/or polymerases, can be attached to the passivated layer for immobilization to the support. In some embodiments, the support comprises a low non-specific binding surface that enable improved nucleic acid hybridization and amplification performance on the support. In some embodiments, the support may comprise one or more layers of a covalently or non-covalently attached low-binding, chemical modification layers, e.g., silane layers, polymer films, and one or more covalently or non-covalently attached oligonucleotides that can be used for immobilizing a plurality of nucleic acid concatemer molecules to the support. In some embodiments, the support can comprise a functionalized polymer coating layer covalently bound at least to a portion of the support via a chemical group on the support, a primer grafted to the functionalized polymer coating, and a water-soluble protective coating on the primer and the functionalized polymer coating. In some embodiments, the functionalized polymer coating comprises a poly(N-(5-azidoacetamidylpentyl)acrylamide-co-acrylamide (PAZAM). In some embodiments, the support comprises a surface coating having at least one hydrophilic polymer coating layer and at least one layer of a plurality of oligonucleotides. In some embodiments, the hydrophilic polymer coating layer can comprise polyethylene glycol (PEG). In some embodiments, the hydrophilic polymer coating layer can comprise branched PEG having at least 4 branches (e.g., 4, 5, 6, 7, 8, 9, 10, or more branches). In some embodiments, the low non-specific binding coating has a degree of hydrophilicity which can be measured as a water contact angle, where the water contact angle is no more than 45 degrees (e.g., no more than 5 degrees, no more than 10 degrees, no more than 15 degrees, no more than degrees, no more than 25 degrees, no more than 30 degrees, no more than 35 degrees, no more than 40 degrees, or no more than 45 degrees). In some embodiments, the density of the concatemer molecules immobilized to the support or immobilized to the coating on the support is about (e.g., about 10², 10³, 10⁴, 10⁵, or 10⁶). In some embodiments, the density of the concatemer molecules immobilized to the support or immobilized to the coating on the support is about 10⁶-10⁹ per mm² (e.g., about 10⁶, 10⁷, 10⁸, or 10). In some embodiments, the density of the concatemer molecules immobilized to the support or immobilized to the coating on the support is about 10⁹-10¹² (e.g., about 10⁹, 10¹⁰, 10¹¹, or 10¹²) per mm². In some embodiments, the plurality of the concatemer molecules is immobilized to the support or immobilized to the coating on the support at pre-determined sites on the support (or the coating on the support). In some embodiments, the plurality of the concatemer molecules is immobilized to the coating on the support at random sites on the support (or the coating on the support).

In some embodiments, the distributing of step (c) can be conducted in the presence of a high-efficiency hybridization buffer which comprises: (i) a first polar aprotic solvent having a dielectric constant that is no greater than 40 (e.g., less than 10, or about 10, 15, 20, or 40) and having a polarity index of 4-9 (e.g., 4, 5, 6, 7, 8, or 9); (ii) a second polar aprotic solvent having a dielectric constant that is no greater than 115 (e.g., less than 10, 10, 15, 20, 40, 50, 75, 100, 105, 105, 110, or 115) and is present in the hybridization buffer formulation in an amount effective to denature double-stranded nucleic acids; (iii) a pH buffer system that maintains the pH of the hybridization buffer formulation in a range of about 4-8 (e.g., 4, 5, 6, 7, or 8); and (iv) a crowding agent in an amount sufficient to enhance or facilitate molecular crowding. In some embodiments, the high efficiency hybridization buffer comprises: (i) the first polar aprotic solvent comprises acetonitrile at 25-50% (e.g., 25%, 30%, 35%, 40%, 45%, or 50%) by volume of the hybridization buffer; (ii) the second polar aprotic solvent comprises formamide at 5-10% (e.g., 5%, 6%, 7%, 8%, 9%, or 10%) by volume of the hybridization buffer; (iii) the pH buffer system comprises 2-(N-morpholino)ethanesulfonic acid (MES) at a pH of 5-6.5 (e.g., about 5.0, 5.5, 6.0, or 6.5); and (iv) the crowding agent comprises polyethylene glycol (PEG) at 5-35% (e.g., 5%, 10%, 15%, 20%, 25%, 30%, or 35%) by volume of the hybridization buffer. In some embodiments, the high efficiency hybridization buffer further comprises betaine.

### In-Solution Rolling Circle Amplification Using Single-Strand Splint Strands Using Circularized Library Molecules Generated Via Ss-Splint Strands

In some embodiments, the methods for conducting rolling circle amplification reaction on a plurality of covalently closed circular library molecules which are hybridized to single-stranded splint strands (800), wherein individual covalently closed circular library molecules (1000) in the plurality comprise a universal binding sequence for a first surface primer, the method comprises (a): contacting in solution the plurality of covalently closed circular library molecules (1000) which are hybridized to single-stranded splint strands (800) with a plurality of strand-displacing polymerases and a plurality of nucleotides under a condition suitable for conducting a first rolling circle amplification reaction using the single-stranded splint strand (800) as an amplification primer thereby generating a plurality of concatemer molecules which are still hybridized to their covalently closed circular library molecules (1000) (e.g., see FIGS. 4-5).

In some embodiments, the methods for conducting rolling circle amplification reaction further comprises step (b): distributing the plurality of concatemer molecules which are hybridized to their covalently closed circular library molecule (1000) onto a support having a plurality of the first surface primers immobilized thereon, under a condition suitable for hybridizing at least a portion of the concatemers to the plurality of the immobilized first surface primers thereby immobilizing the plurality of concatemer molecules. In some embodiments, the plurality of immobilized concatemer molecules is still hybridized to their covalently closed circular library molecules (1000).

In some embodiments, the methods for conducting rolling circle amplification reaction further comprises step (c): contacting the plurality of immobilized concatemer molecules with a plurality of strand-displacing polymerases and a plurality of nucleotides, under a condition suitable to conduct a second rolling circle amplification reaction on the support using the plurality of covalently closed circular library molecules (1000) as template molecules, thereby extending the plurality of immobilized nucleic acid concatemer molecules.

In some embodiments, the first and/or the second rolling circle amplification reactions can be conducted with a plurality of nucleotides which comprise any combination of two or more of dATP, dGTP, dCTP, dTTP and/or dUTP. In some embodiments, individual immobilized concatemers are hybridized to individual first surface primers. In some embodiments, individual covalently closed circular library molecules (1000) in the plurality comprise universal binding sequences for a first and second surface primer (e.g., (720) and (730) respectively) so that the in-solution rolling circle amplification reaction generates concatemer molecules having multiple tandem copies of universal binding sequences for first and second surface primers. In some embodiments, the support further comprises a plurality of second surface primers. In some embodiments, the immobilized second surface primers serve to pin down at least one portion of the concatemer molecules to the support. In some embodiments, the immobilized second surface primers have a non-extendible 3′ end and cannot be used for amplification. In some embodiments, the immobilized concatemers can be subjected to sequencing reactions.

In some embodiments, in the methods for conducting rolling circle amplification reaction, the plurality of the first surface primers immobilized on the support comprise the sequence 5′-GATCAGGTGAGGCTGCGACGACT-3′ (SEQ ID NO:7). In some embodiments, individual first surface primers can hybridize to a covalently closed circular library molecule (1000) having a universal binding sequence for the first surface primer.

In some embodiments, in the methods for conducting rolling circle amplification reaction, the plurality of the first surface primers immobilized on the support comprise the sequence 5′-CAAGCAGAAGACGGCATACGA-3′ (SEQ ID NO:5). In some embodiments, individual first surface primers can hybridize to a covalently closed circular library molecule (1000) having a universal binding sequence for the first surface primer.

In some embodiments, the plurality of the second surface primers immobilized on the support comprise the sequence 5′-CATGTAATGCACGTACTTTCAGGGT-3′ (SEQ ID NO:1 or a complementary sequence thereof). In some embodiments, individual second surface primers can hybridize to a portion of the concatemer molecules having a universal binding sequence for the second surface primer.

In some embodiments, the plurality of the second surface primers immobilized on the support comprise the sequence 5′-AATGATACGGCGACCACCGA-3′ (SEQ ID NO:204 or a complementary sequence thereof). Individual second surface primers can hybridize to a portion of the concatemer molecules having a universal binding sequence for the second surface primer.

In some embodiments the immobilized second surface primers serve to pin down at least one portion of the concatemer molecules to the support. In some embodiments, the immobilized second surface primers have a non-extendible 3′ end and cannot be used amplification. In some embodiments, the immobilized concatemers can be subjected to sequencing reactions.

In some embodiments, the plurality of concatemer molecules of step (b) can be distributed onto a support that is coated with one or more compounds to produce a passivated layer on the support (e.g., FIG. 8). In some embodiments, the passivated layer forms a porous or semi-porous layer. In some embodiments, the surface primer, concatemer template molecule and/or polymerase, can be attached to the passivated layer for immobilization to the support. In some embodiments, the support comprises a low non-specific binding surface that enable improved nucleic acid hybridization and amplification performance on the support. In some embodiments, the support may comprise one or more layers of a covalently or non-covalently attached low-binding, chemical modification layers, e.g., silane layers, polymer films, and one or more covalently or non-covalently attached oligonucleotides that can be used for immobilizing a plurality of nucleic acid concatemer molecules to the support. In some embodiments, the support can comprise a functionalized polymer coating layer covalently bound at least to a portion of the support via a chemical group on the support, a primer grafted to the functionalized polymer coating, and a water-soluble protective coating on the primer and the functionalized polymer coating. In some embodiments, the functionalized polymer coating comprises a poly(N-(5-azidoacetamidylpentyl)acrylamide-co-acrylamide (PAZAM). In some embodiments, the support comprises a surface coating having at least one hydrophilic polymer coating layer and at least one layer of a plurality of oligonucleotides. In some embodiments, the hydrophilic polymer coating layer can comprise polyethylene glycol (PEG). In some embodiments, the hydrophilic polymer coating layer can comprise branched PEG having at least 4 branches (e.g., 4, 5, 6, 7, 8, 9, 10, or more branches). In some embodiments, the low non-specific binding coating has a degree of hydrophilicity which can be measured as a water contact angle, where the water contact angle is no more than 45 degrees (e.g., no more than 5 degrees, no more than 10 degrees, no more than 15 degrees, no more than 20 degrees, no more than 25 degrees, no more than 30 degrees, no more than 35 degrees, no more than 40 degrees, or no more than 45 degrees). In some embodiments, the density of the concatemer molecules immobilized to the support or immobilized to the coating on the support is about 10²-10⁶ per mm² (e.g., about 10², 10³, 10⁴, 10⁵, or 10⁶). In some embodiments, the density of the concatemer molecules immobilized to the support or immobilized to the coating on the support is about 10⁶-10⁹ per mm² (e.g., about 10⁶, 10⁷, 10⁸, or 10⁹). In some embodiments, the density of the concatemer molecules immobilized to the support or immobilized to the coating on the support is about 10⁹-10¹² (e.g., about 10⁹, 10¹⁰, 10¹¹, or 10¹²) per mm². In some embodiments, the plurality of the concatemer molecules is immobilized to the support or immobilized to the coating on the support at pre-determined sites on the support (or the coating on the support). In some embodiments, the plurality of the concatemer molecules is immobilized to the coating on the support at random sites on the support (or the coating on the support).

In some embodiments, the distributing of step (b) can be conducted in the presence of a high-efficiency hybridization buffer which comprises: (i) a first polar aprotic solvent having a dielectric constant that is no greater than 40 (e.g., less than 10, or about 10, 15, 20, or 40) and having a polarity index of 4-9 (e.g., 4, 5, 6, 7, 8, or 9); (ii) a second polar aprotic solvent having a dielectric constant that is no greater than 115 (e.g., less than 10, 10, 15, 20, 40, 50, 75, 100, 105, 105, 110, or 115) and is present in the hybridization buffer formulation in an amount effective to denature double-stranded nucleic acids; (iii) a pH buffer system that maintains the pH of the hybridization buffer formulation in a range of about 4-8 (e.g., 4, 5, 6, 7, or 8); and (iv) a crowding agent in an amount sufficient to enhance or facilitate molecular crowding. In some embodiments, the high efficiency hybridization buffer comprises: (i) the first polar aprotic solvent comprises acetonitrile at 25-50% (e.g., 25%, 30%, 35%, 40%, 45%, or 50%) by volume of the hybridization buffer; (ii) the second polar aprotic solvent comprises formamide at 5-10% (e.g., 5%, 6%, 7%, 8%, 9%, or 10%) by volume of the hybridization buffer; (iii) the pH buffer system comprises 2-(N-morpholino)ethanesulfonic acid (MES) at a pH of 5-6.5 (e.g., about 5.0, 5.5, 6.0, or 6.5); and (iv) the crowding agent comprises polyethylene glycol (PEG) at 5-35% (e.g., 5%, 10%, 15%, 20%, 25%, 30%, or 35%) by volume of the hybridization buffer. In some embodiments, the high efficiency hybridization buffer further comprises betaine.

### Methods for Sequencing

In another aspect, the present disclosure provides methods for sequencing any of the immobilized concatemer molecules described herein. In some embodiments, any of the methods for conducting rolling circle amplification reaction described herein can be used to generate a plurality of concatemer molecules immobilized to a support, and the immobilized concatemers can be subjected to sequencing reactions. In some embodiments, the sequencing reactions employ detectably labeled nucleotide analogs. In some embodiments, the sequencing reactions employ a two-stage sequencing reaction comprising binding detectably labeled multivalent molecules and incorporating nucleotide analogs. The terms concatemer molecule and template molecule are used interchangeably herein.

In some embodiments, the use of at least one reagent to remove deaminated nucleotide bases from the linear library molecules and/or from the circular library molecules, can improve the quality of the sequencing data, e.g., by decreasing the level of low-quality T base calls or A base calls.

In some embodiments, the immobilized concatemer can self-collapse into a compact nucleic acid nanoball. In some embodiments, inclusion of one or more compaction oligonucleotides during the RCA reaction can further compact the size and/or shape of the nanoball. In some embodiments, an increase in the number of tandem repeat units in a given concatemer increases the number of sites along the concatemer for hybridizing to multiple sequencing primers (e.g., sequencing primers having a universal sequence) which serve as multiple initiation sites for polymerase-catalyzed sequencing reactions. In some embodiments, when the sequencing reaction employs detectably labeled nucleotides and/or detectably labeled multivalent molecules (e.g., having nucleotide units), the signals emitted by the nucleotides or nucleotide units that participate in the parallel sequencing reactions along the concatemer yield an increased signal intensity for each concatemer. In certain embodiments, multiple portions of a given concatemer can be simultaneously sequenced. Furthermore, in some embodiments, a plurality of binding complexes can form along a particular concatemer molecule, each binding complex comprising a sequencing polymerase bound to a multivalent molecule. In certain embodiments, the plurality of binding complexes remains stable without dissociation, resulting in increased persistence time, e.g., which increases signal intensity and reduces imaging time.

### Methods for Sequencing Using Nucleotide Analogs

In another aspect, the present disclosure provides methods for sequencing, comprising step (a): contacting a sequencing polymerase to (i) a nucleic acid concatemer molecule and (ii) a nucleic acid primer, wherein the contacting is conducted under a condition suitable to bind the sequencing polymerase to the nucleic acid concatemer molecule which is hybridized to the nucleic acid primer, wherein the nucleic acid concatemer molecule hybridized to the nucleic acid primer forms the nucleic acid duplex. In some embodiments, the sequencing polymerase comprises a recombinant mutant sequencing polymerase. In some embodiments, the primer comprises a 3′ extendible end.

In some embodiments, the methods for sequencing further comprise step (b): contacting the sequencing polymerase with a plurality of nucleotides under a condition suitable for binding at least one nucleotide to the sequencing polymerase which is bound to the nucleic acid duplex and suitable for polymerase-catalyzed nucleotide incorporation. In some embodiments, the sequencing polymerase is contacted with the plurality of nucleotides in the presence of at least one catalytic cation comprising magnesium and/or manganese. In some embodiments, the plurality of nucleotides comprises at least one nucleotide analog having a chain terminating moiety at the sugar 2′ or 3′ position. In some embodiments, the plurality of nucleotides comprises at least one nucleotide that lacks a chain terminating moiety.

In some embodiments, the methods for sequencing further comprise step (c): incorporating at least one nucleotide into the 3′ end of the extendible primer under a condition suitable for incorporating the at least one nucleotide. In some embodiments, the suitable conditions for nucleotide binding the polymerase and for incorporation the nucleotide can be the same or different. In some embodiments, conditions suitable for incorporating the nucleotide comprise inclusion of at least one catalytic cation comprising magnesium and/or manganese. In some embodiments, the at least one nucleotide binds the sequencing polymerase and incorporates into the 3′ end of the extendible primer. In some embodiments, the incorporating the nucleotide into the 3′ end of the primer in step (c) comprises a primer extension reaction.

In some embodiments, the methods for sequencing further comprise step (d): repeating the incorporating at least one nucleotide into the 3′ end of the extendible primer of step (c) at least once. In some embodiments, the plurality of nucleotides comprises a plurality of nucleotides labeled with detectable reporter moiety. In some embodiments, the detectable reporter moiety comprises a fluorophore. In some embodiments, the fluorophore is attached to the nucleotide base. In some embodiments, the fluorophore is attached to the nucleotide base with a linker which is cleavable/removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base. In some embodiments, the method further comprises detecting the at least one incorporated nucleotide at step (c) and/or (d). In some embodiments, the method further comprises identifying the at least one incorporated nucleotide at step (c) and/or (d). In some embodiments, the sequence of the nucleic acid concatemer molecule can be determined by detecting and identifying the nucleotide that binds the sequencing polymerase, thereby determining the sequence of the concatemer molecule. In some embodiments, the sequence of the nucleic acid concatemer molecule can be determined by detecting and identifying the nucleotide that incorporates into the 3′ end of the primer, thereby determining the sequence of the concatemer molecule.

In some embodiments, in the methods for sequencing, the plurality of sequencing polymerases that are bound to the nucleic acid duplexes comprise a plurality of complexed polymerases, having at least a first and second complexed polymerase, wherein (a) the first complexed polymerases comprises a first sequencing polymerase bound to a first nucleic acid duplex comprising a first nucleic acid template sequence which is hybridized to a first nucleic acid primer, (b) the second complexed polymerases comprises a second sequencing polymerase bound to a second nucleic acid duplex comprising a second nucleic acid template sequence which is hybridized to a second nucleic acid primer, (c) the first and second nucleic acid template sequences comprise the same or different sequences, (d) the first and second nucleic acid concatemers are clonally-amplified, (e) the first and second primers comprise extendible 3′ ends or non-extendible 3′ ends, and (f) the plurality of complexed polymerases are immobilized to a support. In some embodiments, the density of the plurality of complexed polymerases is about 10²-10¹⁵ (e.g., 10², 10³, 10⁴, 10⁵, 10⁶, 10⁷, 10⁸, 10⁹, 10¹⁰, 10¹¹, 10¹², 10¹³, 10¹⁴, or 10¹⁵) complexed polymerases per mm² that are immobilized to the support.

### Two-Stage Methods for Nucleic Acid Sequencing

In another aspect, the present disclosure provides a two-stage method for sequencing nucleic acid molecules. In some embodiments, the first stage generally comprises binding multivalent molecules to complexed polymerases to form multivalent-complexed polymerases and detecting the multivalent-complexed polymerases.

In some embodiments, the first stage comprises step (a): contacting a plurality of a first sequencing polymerase to (i) a plurality of nucleic acid concatemer molecules and (ii) a plurality of nucleic acid primers, wherein the contacting is conducted under a condition suitable to bind the plurality of first sequencing polymerases to the plurality of nucleic acid concatemer molecules and the plurality of nucleic acid primers thereby forming a plurality of first complexed polymerases each comprising a first sequencing polymerase bound to a nucleic acid duplex wherein the nucleic acid duplex comprises a nucleic acid concatemer molecule hybridized to a nucleic acid primer. In some embodiments, the first polymerase comprises a recombinant mutant sequencing polymerase.

In some embodiments, in the methods for sequencing concatemer molecules, the primer comprises a 3′ extendible end or a 3′ non-extendible end. In some embodiments, the plurality of nucleic acid concatemer molecules comprise amplified template molecules (e.g., clonally amplified template molecules). In some embodiments, the plurality of nucleic acid concatemer molecules comprise one copy of a target sequence of interest. In some embodiments, the plurality of nucleic acid molecules comprises two or more tandem copies of a target sequence of interest (e.g., concatemers). In some embodiments, the nucleic acid concatemer molecules in the plurality of nucleic acid concatemer molecules comprise the same target sequence of interest or different target sequences of interest. In some embodiments, the plurality of nucleic acid concatemer molecules and/or the plurality of nucleic acid primers are in solution or are immobilized to a support. In some embodiments, when the plurality of nucleic acid concatemer molecules and/or the plurality of nucleic acid primers are immobilized to a support, the binding with the first sequencing polymerase generates a plurality of immobilized first complexed polymerases. In some embodiments, the plurality of nucleic acid concatemer molecules and/or nucleic acid primers are immobilized to 10²-10¹⁵ (e.g., 10², 10³, 10⁴, 10⁵, 10⁶, 10⁷, 10⁸, 10⁹, 10¹⁰, 10¹¹, 10¹², 10¹³, 10¹⁴, or 10¹⁵) different sites on a support. In some embodiments, the binding of the plurality of concatemer molecules and nucleic acid primers with the plurality of first sequencing polymerases generates a plurality of first complexed polymerases immobilized to 10²-10¹⁵ (e.g., 10², 10³, 10⁴, 10⁵, 10⁶, 10⁷, 10⁸, 10⁹, 10¹⁰, 10¹¹, 10¹², 10¹³, 10¹⁴, or 10¹⁵) different sites on the support. In some embodiments, the plurality of immobilized first complexed polymerases on the support are immobilized to pre-determined or to random sites on the support. In some embodiments, the plurality of immobilized first complexed polymerases are in fluid communication with each other to permit flowing a solution of reagents (e.g., enzymes including sequencing polymerases, multivalent molecules, nucleotides, and/or divalent cations) onto the support so that the plurality of immobilized complexed polymerases on the support are reacted with the solution of reagents in a massively parallel manner.

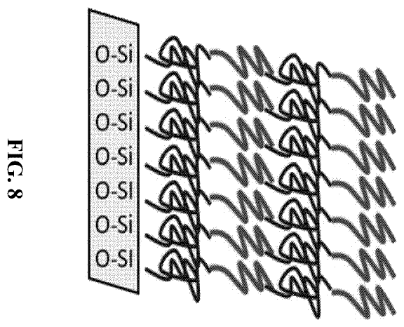

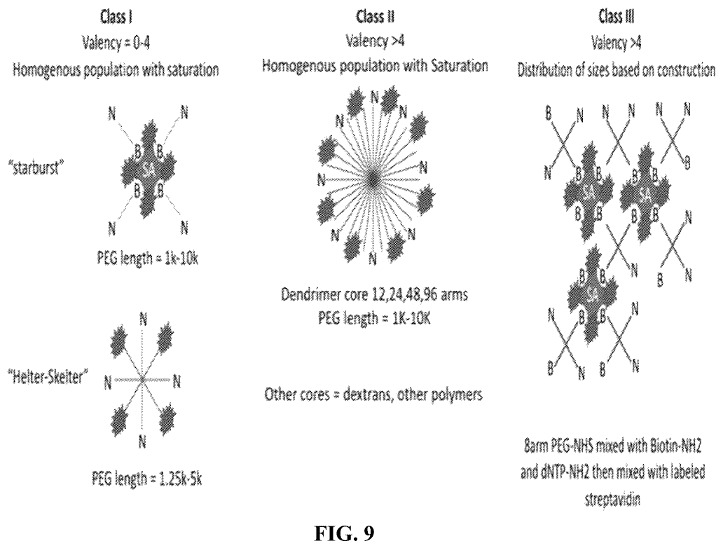

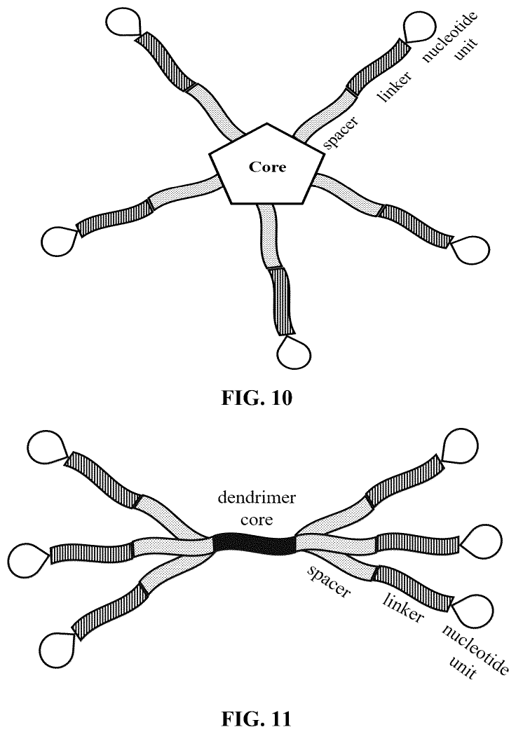

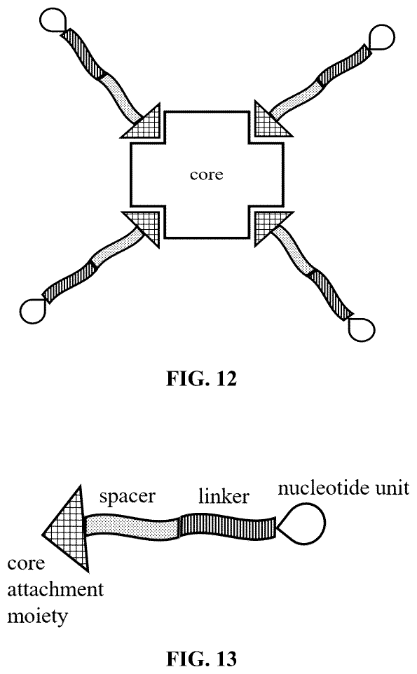

In some embodiments, the methods for sequencing further comprise step (b): contacting the plurality of first complexed polymerases with a plurality of multivalent molecules to form a plurality of multivalent-complexed polymerases (e.g., binding complexes). In some embodiments, individual multivalent molecules in the plurality of multivalent molecules comprise a core attached to multiple nucleotide arms and each nucleotide arm is attached to a nucleotide (e.g., nucleotide unit) (e.g., FIGS. 9-13). In some embodiments, the contacting of step (b) is conducted under a condition suitable for binding complementary nucleotide units of the multivalent molecules to at least two of the plurality of first complexed polymerases thereby forming a plurality of multivalent-complexed polymerases. In some embodiments, the condition is suitable for inhibiting polymerase-catalyzed incorporation of the complementary nucleotide units into the primers of the plurality of multivalent-complexed polymerases. In some embodiments, the plurality of multivalent molecules comprises at least one multivalent molecule having multiple nucleotide arms (e.g., FIGS. 9-13) each attached with a nucleotide analog (e.g., nucleotide analog unit), where the nucleotide analog includes a chain terminating moiety at the sugar 2′ and/or 3′ position. In some embodiments, the plurality of multivalent molecules comprises at least one multivalent molecule comprising multiple nucleotide arms each attached with a nucleotide unit that lacks a chain terminating moiety. In some embodiments, at least one of the multivalent molecules in the plurality of multivalent molecules is labeled with a detectable reporter moiety. In some embodiments, any portion of the multivalent molecule can be labeled including the core, nucleotide arm or nucleo-base. In some embodiments, the detectable reporter moiety comprises a fluorophore. In some embodiments, the contacting of step (b) is conducted in the presence of at least one non-catalytic cation comprising strontium, barium and/or calcium.

In some embodiments, the methods for sequencing further comprise step (c): detecting the plurality of multivalent-complexed polymerases. In some embodiments, the detecting includes detecting the multivalent molecules that are bound to the complexed polymerases, where the complementary nucleotide units of the multivalent molecules are bound to the primers, but incorporation of the complementary nucleotide units is inhibited. In some embodiments, the multivalent molecules are labeled with a detectable reporter moiety to permit detection. In some embodiments, the labeled multivalent molecules comprise a fluorophore attached to the core, linker and/or nucleotide unit of the multivalent molecules.

In some embodiments, the methods for sequencing further comprise step (d): identifying the base of the complementary nucleotide units that are bound to the plurality of first complexed polymerases, thereby determining the sequence of the concatemer molecule. In some embodiments, the multivalent molecules are labeled with a detectable reporter moiety that corresponds to the particular nucleotide units attached to the nucleotide arms to permit identification of the complementary nucleotide units (e.g., nucleotide base adenine, guanine, cytosine, thymine or uracil) that are bound to the plurality of first complexed polymerases.

In some embodiments, the second stage of the two-stage sequencing method generally comprises nucleotide incorporation. In some embodiments, the methods for sequencing further comprise step (e): dissociating the plurality of multivalent-complexed polymerases and removing the plurality of first sequencing polymerases and their bound multivalent molecules and retaining the plurality of nucleic acid duplexes.

In some embodiments, the methods for sequencing further comprises step (f): contacting the plurality of the retained nucleic acid duplexes of step (e) with a plurality of second sequencing polymerases, wherein the contacting is conducted under a condition suitable for binding the plurality of second sequencing polymerases to the plurality of the retained nucleic acid duplexes, thereby forming a plurality of second complexed polymerases each comprising a second sequencing polymerase bound to a nucleic acid duplex. In some embodiments, the second sequencing polymerase comprises a recombinant mutant sequencing polymerase.

In some embodiments, the plurality of first sequencing polymerases of step (a) has an amino acid sequence that is 100% identical to the amino acid sequence as the plurality of the second sequencing polymerases of step (f). In some embodiments, the plurality of first sequencing polymerases of step (a) has an amino acid sequence that differs from the amino acid sequence of the plurality of the second sequencing polymerases of step (f).

In some embodiments, the methods for sequencing further comprise step (g): contacting the plurality of second complexed polymerases with a plurality of nucleotides, wherein the contacting is conducted under a condition suitable for binding complementary nucleotides from the plurality of nucleotides to at least two of the second complexed polymerases thereby forming a plurality of nucleotide-complexed polymerases. In some embodiments, the contacting of step (g) is conducted under a condition that is suitable for promoting polymerase-catalyzed incorporation of the bound complementary nucleotides into the primers of the nucleotide-complexed polymerases thereby forming a plurality of nucleotide-complexed polymerases. In some embodiments, the incorporating the nucleotide into the 3′ end of the primer in step (g) comprises a primer extension reaction. In some embodiments, the contacting of step (g) is conducted in the presence of at least one catalytic cation comprising magnesium and/or manganese. In some embodiments, the contacting of step (g) is conducted in the presence of magnesium and/or manganese. In some embodiments, the plurality of nucleotides comprises native nucleotides (e.g., non-analog nucleotides) or nucleotide analogs. In some embodiments, the plurality of nucleotides comprises a 2′ and/or 3′ chain terminating moiety which is removable or is not removable. In some embodiments, the plurality of nucleotides comprises a plurality of nucleotides labeled with detectable reporter moiety. In some embodiments, the detectable reporter moiety comprises a fluorophore. In some embodiments, the fluorophore is attached to the nucleotide base. In some embodiments, the fluorophore is attached to the nucleotide base with a linker which is cleavable/removable from the base or is not removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base.

In some embodiments, the methods for sequencing further comprise step (h): detecting the complementary nucleotides which are incorporated into the primers of the nucleotide-complexed polymerases. In some embodiments, the plurality of nucleotides is labeled with a detectable reporter moiety to permit detection. In some embodiments, in the methods for sequencing concatemer molecules, the detecting step is omitted.

In some embodiments, the methods for sequencing further comprise step (i): identifying the bases of the complementary nucleotides which are incorporated into the primers of the nucleotide-complexed polymerases. In some embodiments, the identification of the incorporated complementary nucleotides in step (i) can be used to confirm the identity of the complementary nucleotides of the multivalent molecules that are bound to the plurality of first complexed polymerases in step (d). In some embodiments, the identifying of step (i) can be used to determine the sequence of the nucleic acid concatemer molecules. In some embodiments, in the methods for sequencing concatemer molecules, the identifying step is omitted.

In some embodiments, the methods for sequencing further comprise step (j): removing the chain terminating moiety from the incorporated nucleotide when step (g) is conducted by contacting the plurality of second complexed polymerases with a plurality of nucleotides that comprise at least one nucleotide having a 2′ and/or 3′ chain terminating moiety.

In some embodiments, the methods for sequencing further comprise step (k): repeating steps (a)-(j) at least once (e.g., once, twice, three times, four times, five times, or more than six times). In some embodiments, the sequence of the nucleic acid concatemer molecules can be determined by detecting and identifying the multivalent molecules that bind the sequencing polymerases but do not incorporate into the 3′ end of the primer at steps (c) and (d). In some embodiments, the sequence of the nucleic acid concatemer molecule can be determined (or confirmed) by detecting and identifying the nucleotide that incorporates into the 3′ end of the primer at steps (h) and (i).

In some embodiments, in any of the methods for sequencing nucleic acid molecules, the binding of the plurality of first complexed polymerases with the plurality of multivalent molecules forms at least one avidity complex, the method comprising the steps: (a) binding a first nucleic acid primer, a first sequencing polymerase, and a first multivalent molecule to a first portion of a concatemer template molecule thereby forming a first binding complex, wherein a first nucleotide unit of the first multivalent molecule binds to the first sequencing polymerase. (b) binding a second nucleic acid primer, a second sequencing polymerase, and the first multivalent molecule to a second portion of the same concatemer template molecule thereby forming a second binding complex, wherein a second nucleotide unit of the first multivalent molecule binds to the second sequencing polymerase, wherein the first and second binding complexes which include the same multivalent molecule forms an avidity complex. In some embodiments, the first sequencing polymerase comprises any wild type or mutant polymerase described herein. In some embodiments, the second sequencing polymerase comprises any wild type or mutant polymerase described herein. In some embodiments, the concatemer template molecule comprises tandem repeat sequences of a sequence of interest and at least one universal sequencing primer binding site. In some embodiments, the first and second nucleic acid primers can bind to a sequencing primer binding site along the concatemer template molecule. Exemplary multivalent molecules are shown in FIGS. 9-13.

In some embodiments, in any of the methods for sequencing nucleic acid molecules, wherein the method includes binding the plurality of first complexed polymerases with the plurality of multivalent molecules to form at least one avidity complex, the method comprising the steps: (a) contacting the plurality of sequencing polymerases and the plurality of nucleic acid primers with different portions of a concatemer nucleic acid concatemer molecule to form at least first and second complexed polymerases on the same concatemer template molecule; (b) contacting a plurality of multivalent molecules to the at least first and second complexed polymerases on the same concatemer template molecule, under conditions suitable to bind a single multivalent molecule from the plurality to the first and second complexed polymerases, wherein at least a first nucleotide unit of the single multivalent molecule is bound to the first complexed polymerase which includes a first primer hybridized to a first portion of the concatemer template molecule thereby forming a first binding complex (e.g., first ternary complex), and wherein at least a second nucleotide unit of the single multivalent molecule is bound to the second complexed polymerase which includes a second primer hybridized to a second portion of the concatemer template molecule thereby forming a second binding complex (e.g., second ternary complex), wherein the contacting is conducted under a condition suitable to inhibit polymerase-catalyzed incorporation of the bound first and second nucleotide units in the first and second binding complexes, and wherein the first and second binding complexes which are bound to the same multivalent molecule forms an avidity complex; and (c) detecting the first and second binding complexes on the same concatemer template molecule, and (d) identifying the first nucleotide unit in the first binding complex thereby determining the sequence of the first portion of the concatemer template molecule, and identifying the second nucleotide unit in the second binding complex thereby determining the sequence of the second portion of the concatemer template molecule. In some embodiments, the plurality of sequencing polymerases comprise any wild type or mutant sequencing polymerase described herein. The concatemer template molecule comprises tandem repeat sequences of a sequence of interest and at least one universal sequencing primer binding site. In some embodiments, the plurality of nucleic acid primers can bind to a sequencing primer binding site along the concatemer template molecule. Exemplary multivalent molecules are shown in FIGS. 9-13.

### Sequencing-by-Binding

In another aspect, the present disclosure provides methods for sequencing any of the immobilized concatemer molecules described herein. In some embodiments, the sequencing methods comprise a sequencing-by-binding (SBB) procedure which employs non-labeled chain-terminating nucleotides. In some embodiments, the sequencing-by-binding (SBB) method comprises the steps of (a) sequentially contacting a primed template nucleic acid with at least two separate mixtures under ternary complex stabilizing conditions, wherein the at least two separate mixtures each include a polymerase and a nucleotide. In some embodiments, the sequentially contacting results in the primed template nucleic acid being contacted, under the ternary complex stabilizing conditions, with nucleotide cognates for first, second and third base type base types in the template. In some embodiments, the SBB further comprises (b) examining the at least two separate mixtures to determine whether a ternary complex formed. In some embodiments, the SBB further comprises (c) identifying the next correct nucleotide for the primed template nucleic acid molecule, wherein the next correct nucleotide is identified as a cognate of the first, second or third base type if ternary complex is detected in step (b). In certain embodiments, the next correct nucleotide is imputed to be a nucleotide cognate of a fourth base type based on the absence of a ternary complex in step (b). In some embodiments, the SBB further comprises (d) adding a next correct nucleotide to the primer of the primed template nucleic acid after step (b), thereby producing an extended primer. In some embodiments, the SBB further comprises (e) repeating steps (a) through (d) at least once on the primed template nucleic acid that comprises the extended primer. In certain embodiments, each of steps (a), (b), (c), (d), and (e) is performed, e.g., in order. Exemplary sequencing-by-binding methods are described in U.S. Pat. Nos. 10,246,744 and 10,731,141 (where the contents of both patents are hereby incorporated by reference in their entireties).

### Sequencing Polymerases

The present disclosure provides methods for sequencing nucleic acid molecules, where any of the sequencing methods described herein employ at least one type of sequencing polymerase and a plurality of nucleotides or employ at least one type of sequencing polymerase and a plurality of nucleotides and a plurality of multivalent molecules. In some embodiments, the sequencing polymerase(s) is/are capable of incorporating a complementary nucleotide opposite a nucleotide in a concatemer template molecule. In some embodiments, the sequencing polymerase(s) is/are capable of binding a complementary nucleotide unit of a multivalent molecule opposite a nucleotide in a concatemer template molecule. In some embodiments, the plurality of sequencing polymerases comprises recombinant mutant polymerases.

Examples of suitable polymerases for use in sequencing with nucleotides and/or multivalent molecules include but are not limited to: Klenow DNA polymerase; Thermus aquaticus DNA polymerase I (Taq polymerase); KlenTaq polymerase; Candidatus altiarchaeales archaeon; Candidatus Hadarchaeum Yellowstonense; Hadesarchaea archaeon; Euryarchaeota archaeon; Thermoplasmata archaeon; Thermococcus polymerases such as Thermococcus litoralis, bacteriophage T7 DNA polymerase; human alpha, delta and epsilon DNA polymerases; bacteriophage polymerases such as T4, RB69 and phi29 bacteriophage DNA polymerases; Pyrococcus furiosus DNA polymerase (Pfu polymerase); Bacillus subtilis DNA polymerase III; E. coli DNA polymerase III alpha and epsilon; 9 degree N polymerase; reverse transcriptases such as HIV type M or O reverse transcriptases; avian myeloblastosis virus reverse transcriptase; Moloney Murine Leukemia Virus (MMLV) reverse transcriptase; or telomerase. Further non-limiting examples of DNA polymerases include those from various Archaea genera, such as, Aeropyrum, Archaeglobus, Desulfurococcus, Pyrobaculum, Pyrococcus, Pyrolobus, Pyrodictium, Staphylothermus, Stetteria, Sulfolobus, Thermococcus, and Vulcanisaeta and the like or variants thereof, including such polymerases as are known in the art such as 9 degrees N, VENT®, DEEP VENT®, THERMINATOR®, Pfu, KOD, Pfx, Tgo and RB69 polymerases.

### Nucleotides

The present disclosure provides methods for sequencing nucleic acid molecules using nucleotides, wherein at least one nucleotide in the plurality of nucleotides comprise a base, sugar and at least one phosphate group. In some embodiments, at least one nucleotide in the plurality comprises an aromatic base, a five-carbon sugar (e.g., ribose or deoxyribose), and one or more phosphate groups (e.g., about 1, 2, 3, 4, 5, 6, 7, 8, 9, or 10 phosphate groups). The plurality of nucleotides can comprise at least one type of nucleotide selected from a group consisting of dATP, dGTP, dCTP, dTTP and dUTP. The plurality of nucleotides can comprise a mixture of any combination of two or more types of nucleotides selected from a group consisting of dATP, dGTP, dCTP, dTTP, dUTP, and combinations thereof. In some embodiments, at least one nucleotide in the plurality is not a nucleotide analog. In some embodiments, at least one nucleotide in the plurality comprises a nucleotide analog.

In some embodiments, in any of the methods for sequencing nucleic acid molecules described herein, at least one nucleotide in the plurality of nucleotides comprise a chain of one, two or three phosphorus atoms, where the chain is typically attached to the 5′ carbon of the sugar moiety via an ester or phosphoramide linkage. In some embodiments, at least one nucleotide in the plurality is an analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphordithioate, and O-methylphosphoroamidite groups.

In some embodiments, in any of the methods for sequencing nucleic acid molecules described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to the 3′ sugar hydroxyl position, where the sugar comprises a ribose or deoxyribose sugar moiety. In some embodiments, the chain terminating moiety is removable/cleavable from the 3′ sugar hydroxyl position to generate a nucleotide having a 3′OH sugar group. In certain embodiments, the 3′OH sugar group is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the chain terminating moiety is cleavable/removable from the nucleotide, for example and without limitation, by reacting the chain terminating moiety with a chemical agent, pH change, light or heat. In some embodiments, the chain terminating moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thio, and/or disulfide are cleavable with phosphine or with a thiol group, including for example and without limitation, beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride.

In some embodiments, in any of the methods for sequencing nucleic acid molecules described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, in any of the methods for sequencing nucleic acid molecules described herein, the nucleotide comprises a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some embodiments, in any of the methods for sequencing nucleic acid molecules described herein, the plurality of nucleotides comprises a plurality of nucleotides labeled with detectable reporter moiety. In some embodiments, the detectable reporter moiety comprises a fluorophore. In some embodiments, the fluorophore is attached to the nucleotide base. In some embodiments, the fluorophore is attached to the nucleotide base with a linker which is cleavable/removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base.

In some embodiments, in any of the methods for sequencing nucleic acid molecules described herein, the cleavable linker on the nucleotide base comprises a cleavable moiety. In certain embodiments, the cleavable moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the cleavable linker on the base is cleavable/removable from the base by reacting the cleavable moiety with a chemical agent, pH change, light or heat. In some embodiments, the cleavable moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the cleavable moieties aryl and benzyl are cleavable with H2 Pd/C. In some embodiments, the cleavable moieties amine, amide, keto, isocyanate, phosphate, thio, and/or disulfide are cleavable with phosphine or with a thiol group including beta-mercaptoethanol or dithiothritol (DTT). In some embodiments, the cleavable moiety carbonate is cleavable with potassium carbonate (K₂CO₃) in MeOH, with triethylamine in pyridine, or with Zn in acetic acid (AcOH). In some embodiments, the cleavable moieties urea and silyl are cleavable with tetrabutylammonium fluoride, pyridine-HF, ammonium fluoride, or triethylamine trihydrofluoride.

In some embodiments, in any of the methods for sequencing nucleic acid molecules described herein, the cleavable linker on the nucleotide base comprises a cleavable moiety. In certain embodiments, the cleavable moeity includes an azide, azido or azidomethyl group. In some embodiments, the cleavable moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, in any of the methods for sequencing nucleic acid molecules described herein, the chain terminating moiety (e.g., at the sugar 2′ and/or sugar 3′ position) and the cleavable linker on the nucleotide base have the same or different cleavable moieties. In some embodiments, the chain terminating moiety (e.g., at the sugar 2′ and/or sugar 3′ position) and the detectable reporter moiety linked to the base are chemically cleavable/removable with the same chemical agent. In some embodiments, the chain terminating moiety (e.g., at the sugar 2′ and/or sugar 3′ position) and the detectable reporter moiety linked to the base are chemically cleavable/removable with different chemical agents.

### Multivalent Molecules

The present disclosure provides methods for sequencing nucleic acid molecules which employ multivalent molecules. In some embodiments, the multivalent molecule comprises a plurality of nucleotide arms attached to a core and having any configuration including a starburst, helter skelter, or bottle brush configuration (e.g., FIGS. 9-13). In some embodiments, the multivalent molecule comprises: (1) a core; and (2) a plurality of nucleotide arms. In some embodiments, the nucleotide arms comprise (i) a core attachment moiety, (ii) a spacer comprising a PEG moiety, (iii) a linker, and (iv) a nucleotide unit, for example, wherein the core is attached to the plurality of nucleotide arms, wherein the spacer is attached to the linker, and wherein the linker is attached to the nucleotide unit. In some embodiments, the nucleotide unit comprises a base, sugar and at least one phosphate group, and the linker is attached to the nucleotide unit through the base. In some embodiments, the linker comprises an aliphatic chain or an oligo ethylene glycol chain, e.g., with both linker chains having 2-6 (e.g., 2, 3, 4, 5, or 6) subunits. In some embodiments, the linker also includes an aromatic moiety.

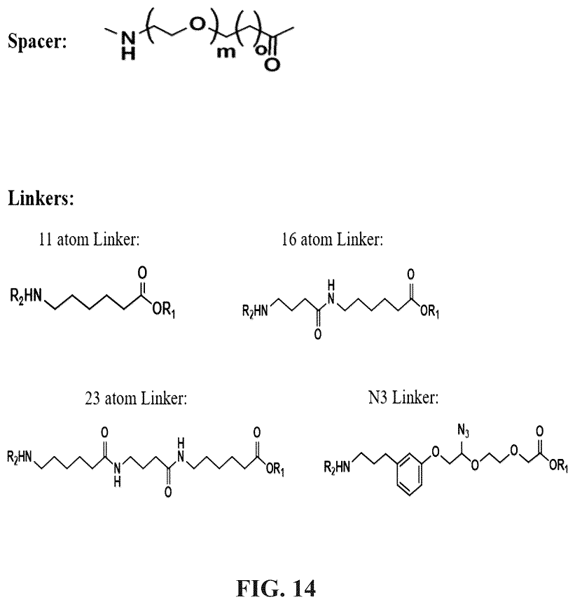

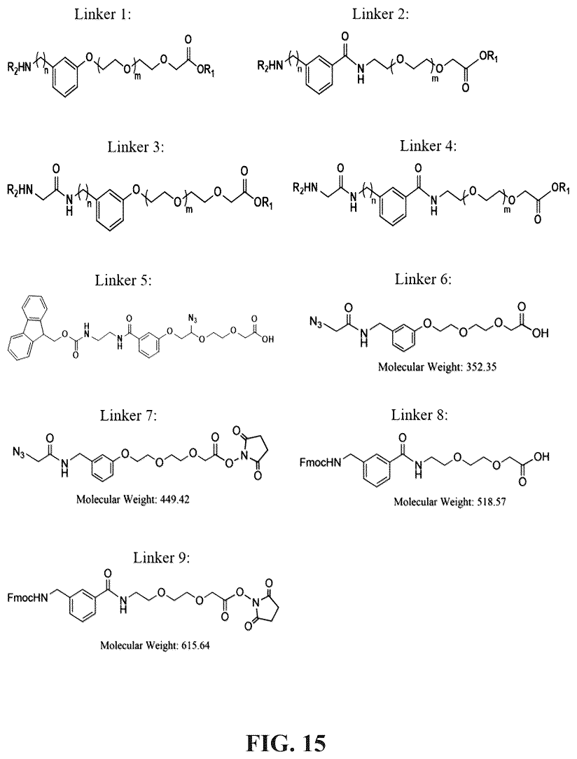

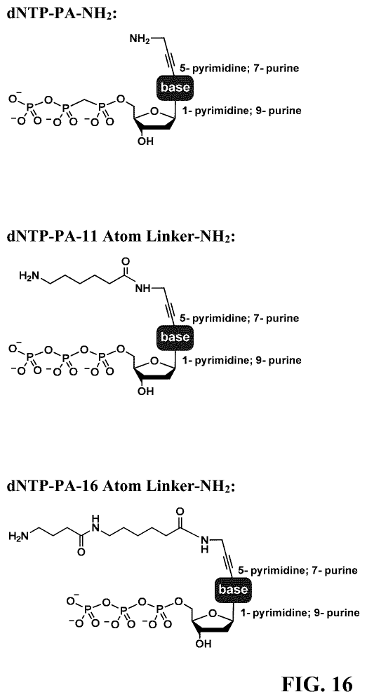

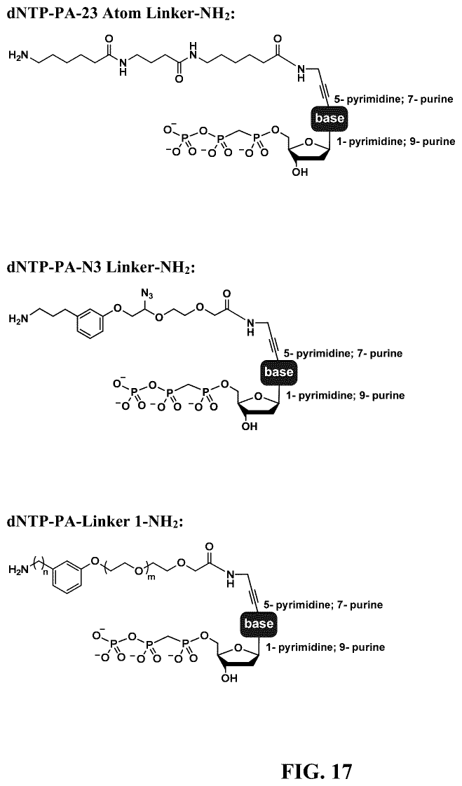

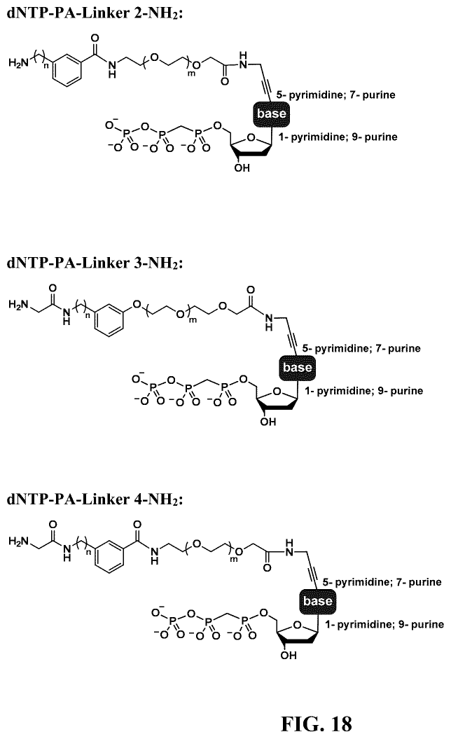

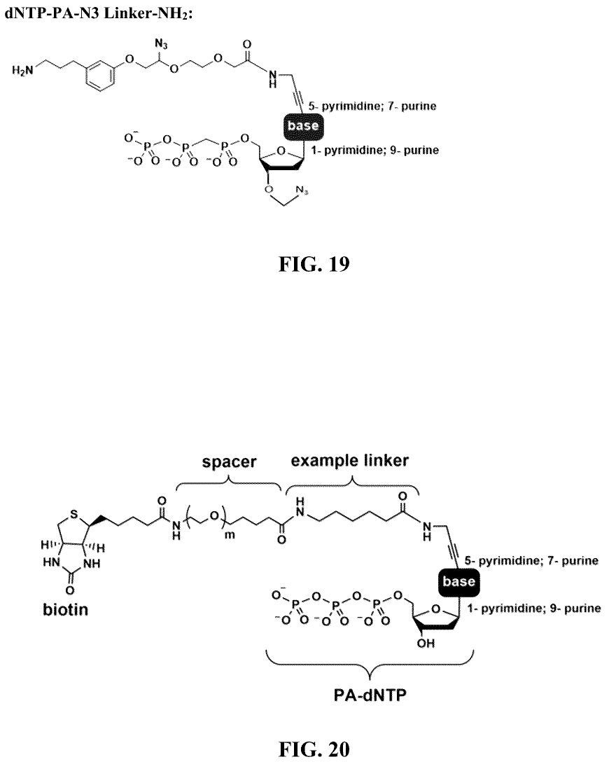

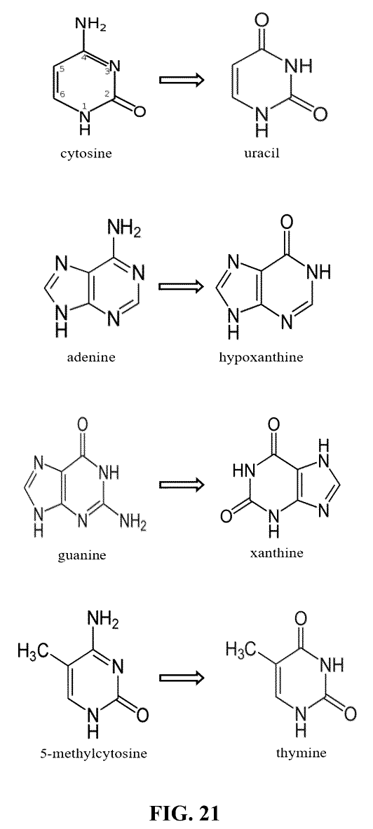

An exemplary nucleotide arm is shown in FIGS. 13 and 20. Exemplary multivalent molecules are shown in FIGS. 9-13. An exemplary spacer is shown in FIG. 14 (top) and exemplary linkers are shown in FIG. 14 (bottom) and FIG. 15. Exemplary nucleotides attached to a linker are shown in FIGS. 16-19. An exemplary biotinylated nucleotide arm is shown in FIG. 20.

In some embodiments, a multivalent molecule comprises a core attached to multiple nucleotide arms. In certain embodiments, the multiple nucleotide arms have the same type of nucleotide unit, which is selected from a group consisting of dATP, dGTP, dCTP, dTTP and dUTP.

In some embodiments, a multivalent molecule comprises a core attached to multiple nucleotide arms, where each arm includes a nucleotide unit. The nucleotide unit comprises an aromatic base, a five-carbon sugar (e.g., ribose or deoxyribose), and one or more phosphate groups (e.g., about 1, 2, 3, 4, 5, 6, 7, 8, 9, or 10 phosphate groups). The plurality of multivalent molecules can comprise one type multivalent molecule having one type of nucleotide unit selected from a group consisting of dATP, dGTP, dCTP, dTTP and dUTP. The plurality of multivalent molecules can comprise a mixture of any combination of two or more types of multivalent molecules, where individual multivalent molecules in the mixture comprise nucleotide units selected from a group consisting of dATP, dGTP, dCTP, dTTP and/or dUTP.

In some embodiments, the nucleotide unit comprises a chain of one, two or three phosphorus atoms, where the chain is typically attached to the 5′ carbon of the sugar moiety via, for example and without limitation, an ester or phosphoramide linkage. In some embodiments, at least one nucleotide unit is a nucleotide analog having a phosphorus chain in which the phosphorus atoms are linked together with intervening O, S, NH, methylene or ethylene. In some embodiments, the phosphorus atoms in the chain include substituted side groups including, for example and without limitation, O, S or BH₃. In some embodiments, the chain includes phosphate groups substituted with analogs, including, for example and without limitation, phosphoramidate, phosphorothioate, phosphordithioate, and O-methylphosphoroamidite groups.

In some embodiments, the multivalent molecule comprises a core attached to multiple nucleotide arms, and wherein individual nucleotide arms comprise a nucleotide unit which is a nucleotide analog having a chain terminating moiety (e.g., blocking moiety) at the sugar 2′ position, at the sugar 3′ position, or at the sugar 2′ and 3′ position. In some embodiments, the nucleotide unit comprises a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to the 3′ sugar hydroxyl position where the sugar comprises a ribose or deoxyribose sugar moiety. In some embodiments, the chain terminating moiety is removable/cleavable from the 3′ sugar hydroxyl position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the chain terminating moiety is cleavable/removable from the nucleotide unit, for example and without limitation, by reacting the chain terminating moiety with a chemical agent, pH change, light or heat. In some embodiments, the chain terminating moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thio, and/or disulfide are cleavable with phosphine or with a thiol group including beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride.

In some embodiments, the nucleotide unit comprises a chain terminating moiety (e.g., blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, the nucleotide unit comprises a chain terminating moiety selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some embodiments, the multivalent molecule comprises a core attached to multiple nucleotide arms, wherein the nucleotide arms comprise a spacer, linker, and nucleotide unit. In some embodiments, the core, linker and/or nucleotide unit is labeled with detectable reporter moiety. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, at least one nucleotide arm of a multivalent molecule has a nucleotide unit that is attached to a detectable reporter moiety. In some embodiments, the detectable reporter moiety is attached to the nucleotide base. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, the core of a multivalent molecule comprises an avidin-like or streptavidin-like moiety and the core attachment moiety comprises biotin. In some embodiments, the core comprises a streptavidin-type or avidin-type moiety which includes an avidin protein, as well as any derivatives, analogs and other non-native forms of avidin, that can bind to at least one biotin moiety. Other forms of avidin moieties include native and recombinant avidin and streptavidin, as well as derivatized molecules, e.g., non-glycosylated avidin and truncated streptavidins. For example, and without limitation, avidin moiety includes de-glycosylated forms of avidin, bacterial streptavidin produced by Streptomyces (e.g., Streptomyces avidinii), as well as derivatized forms, for example, N-acyl avidins, e.g., N-acetyl, N-phthalyl and N-succinyl avidin, and the commercially-available products EXTRAVIDIN™, CAPTAVIDIN™, NEUTRAVIDIN™ and NEUTRALITE AVIDIN™.

In some embodiments, any of the methods for sequencing nucleic acid molecules described herein can include forming a binding complex, where the binding complex comprises (i) a polymerase, a nucleic acid concatemer molecule duplexed with a primer, and a nucleotide, or the binding complex comprises (ii) a polymerase, a nucleic acid concatemer molecule duplexed with a primer, and a nucleotide unit of a multivalent molecule. In some embodiments, the binding complex has a persistence time of greater than about 0.1, 0.2, 0.3, 0.4, 0.5, 0.6, 0.7, 0.9, or 1 second. The binding complex has a persistence time of greater than about 0.1-0.25 seconds, or about 0.25-0.5 seconds, or about 0.5-0.75 seconds, or about 0.75-1 second, or about 1-2 seconds, or about 2-3 seconds, or about 3-4 seconds, or about 4-5 seconds, and/or wherein the method is or may be carried out at a temperature of at or above 15° C., at or above ° C., at or above 25° C., at or above 35° C., at or above 37° C., at or above 42° C. at or above ° C. at or above 60° C., or at or above 72° C., or at or above 80° C., or within a range defined by any of the foregoing.

In some embodiments, the binding complex (e.g., ternary complex) remains stable until subjected to a condition that causes dissociation of interactions between any of the polymerase, template molecule, primer and/or the nucleotide unit or the nucleotide. For example, and without limitation, a dissociating condition comprises contacting the binding complex with any one or any combination of a detergent, EDTA and/or water. In some embodiments, the present disclosure provides said method wherein the binding complex is deposited on, attached to, or hybridized to, a surface showing a contrast to noise ratio in the detecting step of greater than 20. In some embodiments, the present disclosure provides said method wherein the contacting is performed under a condition that stabilizes the binding complex when the nucleotide or nucleotide unit is complementary to a next base of the template nucleic acid and destabilizes the binding complex when the nucleotide or nucleotide unit is not complementary to the next base of the template nucleic acid.

Supports with Low Non-Specific Binding Coatings

In another aspect, the present disclosure provides compositions and methods for use of a support having a plurality of surface primers immobilized thereon, for preparing any of the immobilized concatemers described herein. In some embodiments, the support is passivated with a low non-specific binding coating (e.g., FIG. 8). In some embodiments, the surface coatings described herein exhibit very low non-specific binding to reagents typically used for nucleic acid capture, amplification, and sequencing workflows, e.g., to dyes, nucleotides, enzymes, and nucleic acid primers. In some embodiments, the surface coatings exhibit low background fluorescence signals or high contrast-to-noise (CNR) ratios compared to conventional surface coatings.

In some embodiments, the supports comprise a substrate (or support structure), one or more layers of a covalently or non-covalently attached low-binding, chemical modification layers, e.g., silane layers, polymer films, and one or more covalently or non-covalently attached primer sequences that may be used for tethering single-stranded target nucleic acid(s) to the support surface. In some embodiments, the formulation of the surface, e.g., the chemical composition of one or more layers, the coupling chemistry used to cross-link the one or more layers to the support surface and/or to each other, and the total number of layers, may be varied such that non-specific binding of proteins, nucleic acid molecules, and other hybridization and amplification reaction components to the support surface is minimized or reduced relative to a comparable monolayer. In certain embodiments, the formulation of the surface may be varied such that non-specific hybridization on the support surface is minimized or reduced relative to a comparable monolayer. In certain embodiments, the formulation of the surface may be varied such that non-specific amplification on the support surface is minimized or reduced relative to a comparable monolayer. In certain embodiments, the formulation of the surface may be varied such that specific amplification rates and/or yields on the support surface are maximized. In certain embodiments, amplification levels suitable for detection are achieved in no more than 2, 3, 4, 5, 6, 7, 8, 9, 10, 15, 20, 25, 30, or more than 30 amplification cycles in some embodiments disclosed herein.

In some embodiments, the substrate or support structure that comprises the one or more chemically modified layers, e.g., layers of a low non-specific binding polymer, may be independent or integrated into another structure or assembly. For example, in some embodiments, the substrate or support structure may comprise one or more surfaces within an integrated or assembled microfluidic flow cell. In certain embodiments, the substrate or support structure may comprise one or more surfaces within a microplate format, e.g., the bottom surface of the wells in a microplate. In some embodiments, the substrate or support structure comprises the interior surface (such as the lumen surface) of a capillary. In alternate embodiments, the substrate or support structure comprises the interior surface (such as the lumen surface) of a capillary etched into a planar chip.

In some embodiments, the attachment chemistry used to graft a first chemically modified layer to a surface will generally be dependent on both the material from which the surface is fabricated and the chemical nature of the layer. In some embodiments, the first layer may be covalently attached to the surface. In some embodiments, the first layer may be non-covalently attached, e.g., adsorbed to the surface through non-covalent interactions such as electrostatic interactions, hydrogen bonding, or van der Waals interactions between the surface and the molecular components of the first layer. In either case, the substrate surface may be treated prior to attachment or deposition of the first layer. Any of a variety of surface preparation techniques known to those of skill in the art may be used to clean or treat the surface. For example, and without limitation, glass or silicon surfaces may be acid-washed using a Piranha solution (a mixture of sulfuric acid (H₂SO₄) and hydrogen peroxide (H₂O₂)), base treatment in KOH and NaOH, and/or cleaned using an oxygen plasma treatment method.

Silane chemistries constitute one non-limiting approach for covalently modifying the silanol groups on glass or silicon surfaces to attach more reactive functional groups (e.g., amines or carboxyl groups), which may then be used in coupling linker molecules (e.g., linear hydrocarbon molecules of various lengths, such as C6, C12, C18 hydrocarbons, or linear polyethylene glycol (PEG) molecules) or layer molecules (e.g., branched PEG molecules or other polymers) to the surface. Examples of suitable silanes that may be used in creating any of the disclosed low binding surfaces include, but are not limited to, (3-Aminopropyl) trimethoxysilane (APTMS), (3-Aminopropyl) triethoxysilane (APTES), any of a variety of PEG-silanes (e.g., comprising molecular weights of 1K, 2K, 5K, 10K, 20K, etc.), amino-PEG silane (i.e., comprising a free amino functional group), maleimide-PEG silane, biotin-PEG silane, and the like.

Any of a variety of molecules known to those of skill in the art including, but not limited to, amino acids, peptides, nucleotides, oligonucleotides, other monomers or polymers, or combinations thereof may be used in creating the one or more chemically-modified layers on the surface, where the choice of components used may be varied to alter one or more properties of the surface, e.g., the surface density of functional groups and/or tethered oligonucleotide primers, the hydrophilicity/hydrophobicity of the surface, or the three three-dimensional nature (i.e., “thickness”) of the surface. Examples of suitable polymers that may be used to create one or more layers of low non-specific binding material in any of the disclosed surfaces include, but are not limited to, polyethylene glycol (PEG) of various molecular weights and branching structures, streptavidin, polyacrylamide, polyester, dextran, poly-lysine, and poly-lysine copolymers, or any combination thereof. Examples of conjugation chemistries that may be used to graft one or more layers of material (e.g. polymer layers) to the surface and/or to cross-link the layers to each other include, but are not limited to, biotin-streptavidin interactions (or variations thereof), his tag—Ni/NTA conjugation chemistries, methoxy ether conjugation chemistries, carboxylate conjugation chemistries, amine conjugation chemistries, NHS esters, maleimides, thiol, epoxy, azide, hydrazide, alkyne, isocyanate, and silane.

In some embodiments, the low non-specific binding surface coating may be applied uniformly across the substrate. Alternately, in some embodiments, the surface coating may be patterned, such that the chemical modification layers are confined to one or more discrete regions of the substrate. For example, in some embodiments, the surface may be patterned using photolithographic techniques to create an ordered array or random pattern of chemically modified regions on the surface. Alternately or in combination, in some embodiments, the substrate surface may be patterned using, e.g., contact printing and/or ink-jet printing techniques. In some embodiments, an ordered array or random pattern of chemically modified regions may comprise at least 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10,000 or more discrete regions.

In some embodiments, in order to achieve low nonspecific binding surfaces, hydrophilic polymers may be nonspecifically adsorbed or covalently grafted to the surface. Typically, passivation is performed utilizing poly(ethylene glycol) (PEG, also known as polyethylene oxide (PEO) or polyoxyethylene) or other hydrophilic polymers with different molecular weights and end groups that are linked to a surface using, for example and without limitation, silane chemistry. In some embodiments, the end groups distal from the surface can include, but are not limited to, biotin, methoxy ether, carboxylate, amine, NHS ester, maleimide, and bis-silane. In some embodiments, two or more layers of a hydrophilic polymer, e.g., a linear polymer, branched polymer, or multi-branched polymer, may be deposited on the surface. In some embodiments, two or more layers may be covalently coupled to each other or internally cross-linked to improve the stability of the resulting surface. In some embodiments, oligonucleotide primers with different base sequences and base modifications (or other biomolecules, e.g., enzymes or antibodies) may be tethered to the resulting surface layer at various surface densities. In some embodiments, for example, both surface functional group density and oligonucleotide concentration may be varied to target a certain primer density range. Additionally, in some embodiments, primer density can be controlled by diluting oligonucleotide with other molecules that carry the same functional group. For example, and without limitation, amine-labeled oligonucleotide can be diluted with amine-labeled polyethylene glycol in a reaction with an NHS-ester coated surface to reduce the final primer density. In some embodiments, primers with different lengths of linkers between the hybridization region and the surface attachment functional group can also be applied to control surface density. Examples of suitable linkers include poly-T and poly-A strands at the 5′ end of the primer (e.g., 0 to 20 bases), PEG linkers (e.g., 3 to 20 monomer units), and carbon-chain (e.g., C6, C12, C18, etc.). To measure the primer density, in some embodiments, fluorescently labeled primers may be tethered to the surface and a fluorescence reading then compared with that for a dye solution of known concentration.

In order to scale primer surface density and add additional dimensionality to hydrophilic or amphoteric surfaces, surfaces comprising multi-layer coatings of PEG and other hydrophilic polymers have been developed. In some embodiments, by using hydrophilic and amphoteric surface layering approaches that include, but are not limited to, the polymer/co-polymer materials described below, it is possible to increase primer loading density on the surface significantly. Traditional PEG coating approaches use monolayer primer deposition, which have been generally reported for single molecule applications, but do not yield high copy numbers for nucleic acid amplification applications. As described herein “layering” can be accomplished using traditional crosslinking approaches with any compatible polymer or monomer subunits such that a surface comprising two or more highly crosslinked layers can be built sequentially. Examples of suitable polymers include, but are not limited to, streptavidin, poly acrylamide, polyester, dextran, poly-lysine, and copolymers of poly-lysine and PEG. In some embodiments, the different layers may be attached to each other through any of a variety of conjugation reactions including, but not limited to, biotin-streptavidin binding, azide-alkyne click reaction, amine-NHS ester reaction, thiol-maleimide reaction, and ionic interactions between positively charged polymer and negatively charged polymer. In some embodiments, high primer density materials may be constructed in solution and subsequently layered onto the surface in multiple steps.

In some embodiments, low non-specific binding coatings exhibit reduced non-specific binding of proteins, nucleic acids, and other components of the hybridization and/or amplification formulation used for solid-phase nucleic acid amplification. In some embodiments, the degree of non-specific binding exhibited by a given support surface may be assessed either qualitatively or quantitatively. For example, in some embodiments, exposure of the surface to fluorescent dyes (e.g., cyanine dyes such as Cy3, or Cy5, etc., fluoresceins, coumarins, rhodamines, etc. or other dyes disclosed herein), fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a qualitative tool for comparison of non-specific binding on supports comprising different surface formulations. In some embodiments, exposure of the surface to fluorescent dyes, fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a quantitative tool for comparison of non-specific binding on supports comprising different surface formulations—provided that care has been taken to ensure that the fluorescence imaging is performed under a condition where fluorescence signal is linearly related (or related in a predictable manner) to the number of fluorophores on the support surface (e.g., under a condition where signal saturation and/or self-quenching of the fluorophore is not an issue) and suitable calibration standards are used. In some embodiments, other techniques known to those of skill in the art, for example and without limitation, radioisotope labeling and counting methods may be used for quantitative assessment of the degree to which non-specific binding is exhibited by the different support surface formulations of the present disclosure.

In some embodiments, some surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. In some embodiments, some surfaces disclosed herein exhibit a ratio of specific to nonspecific fluorescence of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

As noted, in some embodiments, the degree of non-specific binding exhibited by the disclosed low-binding supports may be assessed using a standardized protocol for contacting the surface with a labeled protein (e.g., bovine serum albumin (BSA), streptavidin, a DNA polymerase, a reverse transcriptase, a helicase, a single-stranded binding protein (SSB), etc., or any combination thereof), a labeled nucleotide, a labeled oligonucleotide, etc., under a standardized set of incubation and rinse conditions. In some embodiments, the contacting is followed by detection of the amount of label remaining on the surface and comparison of the signal resulting therefrom to an appropriate calibration standard. In some embodiments, the label may comprise a fluorescent label. In some embodiments, the label may comprise a radioisotope. In some embodiments, the label may comprise any other detectable label known to one of skill in the art. In some embodiments, the degree of non-specific binding exhibited by a given support surface formulation may thus be assessed in terms of the number of non-specifically bound protein molecules (or other molecules) per unit area. In some embodiments, the low-binding supports of the present disclosure may exhibit non-specific protein binding (or non-specific binding of other specified molecules, (e.g., cyanine dyes such as Cy3, or Cy5, etc., fluoresceins, coumarins, rhodamines, etc. or other dyes disclosed herein)) of less than 0.001 molecule per μm², less than 0.01 molecule per μm², less than 0.1 molecule per μm², less than molecule per μm², less than 0.5 molecule per μm², less than 1 molecule per μm², less than molecules per μm², less than 100 molecules per μm², or less than 1,000 molecules per μm². Those of skill in the art will realize that a given support surface of the present disclosure may exhibit non-specific binding falling anywhere within this range, for example, of less than 86 molecules per μm².

For example, and without limitation, some modified surfaces disclosed herein exhibit nonspecific protein binding of less than 0.5 molecule/μm 2 following contact with a 1 μM solution of Cy3 labeled streptavidin (GE Amersham™) in phosphate buffered saline (PBS) buffer for 15 minutes and followed by 3 rinses with deionized water. In some embodiments, some modified surfaces disclosed herein exhibit nonspecific binding of Cy3 dye molecules of less than 0.25 molecules per μm². In some embodiments of independent nonspecific binding assays, 1 μM labeled Cy3 SA (ThermoFisher), 1 μM Cy5 SA dye (ThermoFisher), 10 μM Aminoallyl-dUTP—ATTO-647N (Jena Biosciences), 10 μM Aminoallyl-dUTP—ATTO-Rho11 (Jena Biosciences), 10 μM Aminoallyl-dUTP—ATTO-Rho11 (Jena Biosciences), 10 μM 7-Propargylamino-7-deaza-dGTP—Cy5 (Jena Biosciences, and 10 μM 7-Propargylamino-7-deaza-dGTP—Cy3 (Jena Biosciences) are incubated on low binding substrates at 37° C., e.g., for 15 minutes, in a 384 well plate format. In certain embodiments, each well is rinsed 2-3× with 50 μL deionized RNase/DNase Free water and 2-3× with 25 mM ACES buffer at pH of about 7.4. The 384 well plates may then be imaged on a GE Typhoon instrument using the Cy3, AF555, or Cy5 filter sets (according to the dye test performed) and as specified by the manufacturer's instructions, at a PMT gain setting of 800 and resolution of 50-100 μm. For higher resolution imaging, images may be collected, for example and without limitation, on an Olympus IX83 microscope (Olympus Corp., Center Valley, PA) with a total internal reflectance fluorescence (TIRF) objective lens (100×, 1.5 NA, Olympus), a CCD camera (e.g., an Olympus EM-CCD monochrome camera, Olympus XM-10 monochrome camera, or an Olympus DP80 color and monochrome camera), an illumination source (e.g., an Olympus 100 W Hg lamp, an Olympus 75 W Xe lamp, or an Olympus U-HGLGPS fluorescence light source), and excitation wavelengths of 532 nm or 635 nm. In some embodiments, dichroic mirrors may be purchased from Semrock (IDEX Health & Science, LLC, Rochester, New York), e.g., 405, 488, 532, or 633 nm dichroic reflectors/beamsplitters, and band pass filters chosen as 532 LP or 645 LP concordant with the appropriate excitation wavelength. In some embodiments, some modified surfaces disclosed herein exhibit nonspecific binding of dye molecules of less than 0.25 molecules per μm².

In some embodiments, the surfaces disclosed herein exhibit a ratio of specific to nonspecific binding of a fluorophore (such as Cy3) of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. In some embodiments, the surfaces disclosed herein exhibit a ratio of specific to nonspecific fluorescence signals for a fluorophore (such as Cy3) of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The low-background surfaces consistent with the disclosure herein may exhibit specific dye attachment (e.g., Cy3 attachment) to non-specific dye adsorption (e.g., Cy3 dye adsorption) ratios of at least 4:1, 5:1, 6:1, 7:1, 8:1, 9:1, 10:1, 15:1, 20:1, 30:1, 40:1, 50:1, or more than 50 specific dye molecules attached per molecule nonspecifically adsorbed. Similarly, when subjected to an excitation energy, low-background surfaces consistent with the disclosure herein to which fluorophores, e.g., Cy3, have been attached may exhibit ratios of specific fluorescence signal (e.g., arising from Cy3-labeled oligonucleotides attached to the surface) to non-specific adsorbed dye fluorescence signals of at least 4:1, 5:1, 6:1, 7:1, 8:1, 9:1, 15:1, 20:1, 30:1, 40:1, 50:1, or more than 50:1.

In some embodiments, the degree of hydrophilicity (or “wettability” with aqueous solutions) of the disclosed support surfaces may be assessed, for example, through the measurement of water contact angles in which a small droplet of water is placed on the surface and its angle of contact with the surface is measured using, e.g., an optical tensiometer. In some embodiments, a static contact angle may be determined. In some embodiments, an advancing or receding contact angle may be determined. In some embodiments, the water contact angle for the hydrophilic, low-binding support surfaced disclosed herein may range from about 0 degrees to about 30 degrees. In some embodiments, the water contact angle for the hydrophilic, low-binding support surfaced disclosed herein may no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases the contact angle is no more than 40 degrees. Those of skill in the art will realize that a given hydrophilic, low-binding support surface of the present disclosure may exhibit a water contact angle having a value of anywhere within this range.

In some embodiments, the hydrophilic surfaces disclosed herein facilitate reduced wash times for bioassays, often due to reduced nonspecific binding of biomolecules to the low-binding surfaces. In some embodiments, adequate wash steps may be performed in less than 50, 40, 30, 20, 15, 10, or less than 10 seconds. For example, in some embodiments adequate wash steps may be performed in less than 30 seconds.

The low-binding surfaces of the present disclosure exhibit significant improvement in stability or durability to prolonged exposure to solvents and elevated temperatures, or to repeated cycles of solvent exposure or changes in temperature. For example, in some embodiments, the stability of the disclosed surfaces may be tested by fluorescently labeling a functional group on the surface, or a tethered biomolecule (e.g., an oligonucleotide primer) on the surface, and monitoring fluorescence signal before, during, and after prolonged exposure to solvents and elevated temperatures, or to repeated cycles of solvent exposure or changes in temperature. In some embodiments, the degree of change in the fluorescence used to assess the quality of the surface may be less than 1%, 2%, 3%, 4%, 5%, 10%, 15%, 20%, or 25% over a time period of 1 minute, 2 minutes, 3 minutes, 4 minutes, 5 minutes, 10 minutes, 20 minutes, minutes, 40 minutes, 50 minutes, 60 minutes, 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, hours, 50 hours, or 100 hours of exposure to solvents and/or elevated temperatures (or any combination of these percentages as measured over these time periods). In some embodiments, the degree of change in the fluorescence used to assess the quality of the surface may be less than 1%, 2%, 3%, 4%, 5%, 10%, 15%, 20%, or 25% over 5 cycles, 10 cycles, 20 cycles, 30 cycles, 40 cycles, 50 cycles, 60 cycles, 70 cycles, 80 cycles, 90 cycles, 100 cycles, 200 cycles, 300 cycles, 400 cycles, 500 cycles, 600 cycles, 700 cycles, 800 cycles, 900 cycles, or 1,000 cycles of repeated exposure to solvent changes and/or changes in temperature (or any combination of these percentages as measured over this range of cycles).

In some embodiments, the surfaces disclosed herein may exhibit a high ratio of specific signal to nonspecific signal or other background. For example, when used for nucleic acid amplification, some surfaces may exhibit an amplification signal that is at least 4, 5, 6, 7, 8, 9, 10, 15, 20, 30, 40, 50, 75, 100, or greater than 100-fold greater than a signal of an adjacent unpopulated region of the surface. Similarly, some surfaces exhibit an amplification signal that is at least 4, 5, 6, 7, 8, 9, 10, 15, 20, 30, 40, 50, 75, 100, or greater than 100-fold greater than a signal of an adjacent amplified nucleic acid population region of the surface.

In some embodiments, fluorescence images of the disclosed low background surfaces when used in nucleic acid hybridization or amplification applications to create clusters of hybridized or clonally-amplified nucleic acid molecules (e.g., that have been directly or indirectly labeled with a fluorophore) exhibit contrast-to-noise ratios (CNRs) of at least 10, 20, 40, 50, 60, 70, 80, 90, 100, 110, 120, 130, 140, 150, 160, 170, 180, 190, 20, 210, 220, 230, 240, 250, or greater than 250.

In some embodiments, one or more types of primer (e.g., capture primers) may be attached or tethered to the support surface. In some embodiments, one or more types of adapters may be attached or tethered to the support surface. In some embodiments, the one or more types of adapters or primers may comprise spacer sequences, adapter sequences for hybridization to adapter-ligated target library nucleic acid sequences, forward amplification primers, reverse amplification primers, sequencing primers, and/or molecular barcoding sequences, or any combination thereof. In some embodiments, 1 primer or adapter sequence may be tethered to at least one layer of the surface. In some embodiments, at least 2, 3, 4, 5, 6, 7, 8, 9, 10, or more than 10 different primer or adapter sequences may be tethered to at least one layer of the surface.

In some embodiments, the tethered adapter and/or primer sequences may range in length from about 10 nucleotides to about 100 nucleotides. In some embodiments, the tethered adapter and/or primer sequences may be at least 10, at least 20, at least 30, at least 40, at least at least 60, at least 70, at least 80, at least 90, or at least 100 nucleotides in length. In some embodiments, the tethered adapter and/or primer sequences may be at most 100, at most 90, at most 80, at most 70, at most 60, at most 50, at most 40, at most 30, at most 20, or at most 10 nucleotides in length. Any of the lower and upper values described in this paragraph may be combined to form a range included within the present disclosure, for example, in some embodiments the length of the tethered adapter and/or primer sequences may range from about 20 nucleotides to about 80 nucleotides. Those of skill in the art will recognize that the length of the tethered adapter and/or primer sequences may have any value within this range, e.g., about 24 nucleotides.

In some embodiments, the resultant surface density of primers on the low binding support surfaces of the present disclosure may range from about 100 primer molecules per μm² to about 100,000 primer molecules per μm². In some embodiments, the resultant surface density of primers on the low binding support surfaces of the present disclosure may range from about 100,000 primer molecules per μm² to about 10¹⁵ primer molecules per μm². In some embodiments, the surface density of primers may be at least 1,000, at least 10,000, at least 100,000, or at least 10¹⁵ primer molecules per μm². In some embodiments, the surface density of primers may be at most 10,000, at most 100,000, at most 1,000,000, or at most 10¹⁵ primer molecules per μm². Any of the lower and upper values described in this paragraph may be combined to form a range included within the present disclosure, for example, in some embodiments the surface density of primers may range from about 10,000 molecules per μm² to about 10¹⁵ molecules per μm². Those of skill in the art will recognize that the surface density of primer molecules may have any value within this range, e.g., about 455,000 molecules per μm². In some embodiments, the surface density of target library nucleic acid sequences initially hybridized to adapter or primer sequences on the support surface may be less than or equal to that indicated for the surface density of tethered primers. In some embodiments, the surface density of clonally amplified target library nucleic acid sequences hybridized to adapter or primer sequences on the support surface may span the same range as that indicated for the surface density of tethered primers.

Local densities as listed above do not preclude variation in density across a surface, such that a surface may comprise a region having an oligo density of, for example, 500,000 per μm², while also comprising at least a second region having a substantially different local density.

In some embodiments, the low non-specific binding coating comprise one or more layers of a multi-layered surface coating may comprise a branched polymer or may be linear. Examples of suitable branched polymers include, but are not limited to, branched PEG, branched poly(vinyl alcohol) (branched PVA), branched poly(vinyl pyridine), branched poly(vinyl pyrrolidone) (branched PVP), branched), poly(acrylic acid) (branched PAA), branched polyacrylamide, branched poly(N-isopropylacrylamide) (branched PNIPAM), branched poly(methyl methacrylate) (branched PMA), branched poly(2-hydroxylethyl methacrylate) (branched PHEMA), branched poly(oligo(ethylene glycol) methyl ether methacrylate) (branched POEGMA), branched polyglutamic acid (branched PGA), branched poly-lysine, branched poly-glucoside, and dextran.

In some embodiments, the branched polymers used to create one or more layers of any of the multi-layered surfaces disclosed herein may comprise at least 4 branches, at least 5 branches, at least 6 branches, at least 7 branches, at least 8 branches, at least 9 branches, at least 10 branches, at least 12 branches, at least 14 branches, at least 16 branches, at least 18 branches, at least 20 branches, at least 22 branches, at least 24 branches, at least 26 branches, at least 28 branches, at least 30 branches, at least 32 branches, at least 34 branches, at least 36 branches, at least 38 branches, or at least 40 branches.

Linear, branched, or multi-branched polymers used to create one or more layers of any of the multi-layered surfaces disclosed herein may have a molecular weight of at least 500, at least 1,000, at least 2,000, at least 3,000, at least 4,000, at least 5,000, at least 10,000, at least 15,000, at least 20,000, at least 25,000, at least 30,000, at least 35,000, at least 40,000, at least 45,000, or at least 50,000 daltons.

In some embodiments, e.g., wherein at least one layer of a multi-layered surface comprises a branched polymer, the number of covalent bonds between a branched polymer molecule of the layer being deposited and molecules of the previous layer may range from about one covalent linkage per molecule to about 32 covalent linkages per molecule. In some embodiments, the number of covalent bonds between a branched polymer molecule of the new layer and molecules of the previous layer may be at least 1, at least 2, at least 3, at least 4, at least 5, at least 6, at least 7, at least 8, at least 9, at least 10, at least 12, at least 14, at least 16, at least 18, at least 20, at least 22, at least 24, at least 26, at least 28, at least 30, or at least 32 covalent linkages per molecule.

In some embodiments, any reactive functional groups that remain following the coupling of a material layer to the surface may optionally be blocked by coupling a small, inert molecule using a high yield coupling chemistry. For example, in the case that amine coupling chemistry is used to attach a new material layer to the previous one, any residual amine groups may subsequently be acetylated or deactivated by coupling with a small amino acid such as glycine.

The number of layers of low non-specific binding material, e.g., a hydrophilic polymer material, deposited on the surface, may range from 1 to about 10. In some embodiments, the number of layers is at least 1, at least 2, at least 3, at least 4, at least 5, at least 6, at least 7, at least 8, at least 9, or at least 10. In some embodiments, the number of layers may be at most 10, at most 9, at most 8, at most 7, at most 6, at most 5, at most 4, at most 3, at most 2, or at most 1. Any of the lower and upper values described in this paragraph may be combined to form a range included within the present disclosure, for example, in some embodiments the number of layers may range from about 2 to about 4. In some embodiments, all of the layers may comprise the same material. In some embodiments, each layer may comprise a different material. In some embodiments, the plurality of layers may comprise a plurality of materials. In some embodiments at least one layer may comprise a branched polymer. In some embodiment, all of the layers may comprise a branched polymer.

In some embodiments, one or more layers of low non-specific binding material may in some cases be deposited on and/or conjugated to the substrate surface using a polar protic solvent, a polar or polar aprotic solvent, a nonpolar solvent, or any combination thereof. In some embodiments the solvent used for layer deposition and/or coupling may comprise an alcohol (e.g., methanol, ethanol, propanol, etc.), another organic solvent (e.g., acetonitrile, dimethyl sulfoxide (DMSO), dimethyl formamide (DMF), etc.), water, an aqueous buffer solution (e.g., phosphate buffer, phosphate buffered saline, 3-(N-morpholino)propanesulfonic acid (MOPS), etc.), or any combination thereof. In some embodiments, an organic component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of water or an aqueous buffer solution. In some embodiments, an aqueous component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of an organic solvent. The pH of the solvent mixture used may be less than 6, about 6, 6.5, 7, 7.5, 8, 8.5, 9, or greater than pH 9.

In some embodiments, fluorescence imaging may be performed using any of a variety of fluorophores, fluorescence imaging techniques, and fluorescence imaging instruments known to those of skill in the art. Examples of suitable fluorescence dyes that may be used (e.g., by conjugation to nucleotides, oligonucleotides, or proteins) include, but are not limited to, fluorescein, rhodamine, coumarin, cyanine, and derivatives thereof, including the cyanine derivatives Cyanine dye-3 (Cy3), Cyanine dye-5 (Cy5), Cyanine dye-7 (Cy7), etc. Examples of fluorescence imaging techniques that may be used include, but are not limited to, fluorescence microscopy imaging, fluorescence confocal imaging, two-photon fluorescence, and the like. Examples of fluorescence imaging instruments that may be used include, but are not limited to, fluorescence microscopes equipped with an image sensor or camera, confocal fluorescence microscopes, two-photon fluorescence microscopes, or custom instruments that comprise a suitable selection of light sources, lenses, mirrors, prisms, dichroic reflectors, apertures, and image sensors or cameras, etc. A non-limiting example of a fluorescence microscope equipped for acquiring images of the disclosed low-binding support surfaces and clonally-amplified colonies (polonies) of template nucleic acid sequences hybridized thereon is the Olympus IX83 inverted fluorescence microscope equipped with) 20×, 0.75 NA, a 532 nm light source, a bandpass and dichroic mirror filter set optimized for 532 nm long-pass excitation and Cy3 fluorescence emission filter, a Semrock 532 nm dichroic reflector, and a camera (Andor sCMOS, Zyla 4.2) where the excitation light intensity is adjusted to avoid signal saturation. In some embodiments, the support surface may be immersed in a buffer (e.g., 25 mM ACES, pH 7.4 buffer) while the image is acquired.

In some embodiments, the performance of nucleic acid hybridization and/or amplification reactions using the disclosed reaction formulations and low non-specific binding supports may be assessed using fluorescence imaging techniques, where the contrast-to-noise ratio (CNR) of the images provides a key metric in assessing amplification specificity and non-specific binding on the support. In some embodiments, CNR is defined as: CNR=(Signal−Background)/Noise. In some embodiments, the background term is taken to be the signal measured for the interstitial regions surrounding a particular feature (diffraction limited spot, DLS) in a specified region of interest (ROI). While signal-to-noise ratio (SNR) is often considered to be a benchmark of overall signal quality, in some embodiments, it can be shown that improved CNR can provide a significant advantage over SNR as a benchmark for signal quality in applications that require rapid image capture (e.g., sequencing applications for which cycle times must be minimized), as shown in the example below. In some embodiments, the surfaces of the instant disclosure are also provided in International Application Serial No. PCT/US2019/061556, which is hereby incorporated by reference in its entirety.

In some embodiments of ensemble-based sequencing approaches, the background term is typically measured as the signal associated with ‘interstitial’ regions. In some embodiments, in addition to “interstitial” background (Bᵢₙₜₑᵣ), “intrastitial” background (Bᵢₙₜᵣₐ) exists within the region occupied by an amplified DNA colony. In some embodiments, the combination of these two background signals dictates the achievable CNR, and subsequently directly impacts the optical instrument requirements, architecture costs, reagent costs, run-times, cost/genome, and ultimately the accuracy and data quality for cyclic array-based sequencing applications. In certain embodiments, the Bᵢₙₜₑᵣ background signal arises from a variety of sources; for example, and without limitation, including auto-fluorescence from consumable flow cells, non-specific adsorption of detection molecules that yield spurious fluorescence signals that may obscure the signal from the ROI, the presence of non-specific DNA amplification products (e.g., those arising from primer dimers). In some embodiments of typical next generation sequencing (NGS) applications, this background signal in the current field-of-view (FOV) is averaged over time and subtracted. In some embodiments, the signal arising from individual DNA colonies (i.e., (S)−Bᵢₙₜₑᵣ in the FOV) yields a discernable feature that can be classified. In some embodiments, the intrastitial background (Bᵢₙₜᵣₐ) can contribute a confounding fluorescence signal that is not specific to the target of interest but is present in the same ROI thus making it far more difficult to average and subtract.

In some embodiments, the implementation of nucleic acid amplification on the low-binding substrates of the present disclosure may decrease the Bᵢₙₜₑᵣ background signal by reducing non-specific binding, may lead to improvements in specific nucleic acid amplification, and may lead to a decrease in non-specific amplification that can impact the background signal arising from both the interstitial and intrastitial regions. In some embodiments, the disclosed low-binding support surfaces, optionally used in combination with the disclosed hybridization buffer formulations, may lead to improvements in CNR by a factor of 2, 5, 10, 100, or 1000-fold over those achieved using conventional supports and hybridization, amplification, and/or sequencing protocols. Although described here in the context of using fluorescence imaging as the read-out or detection mode, it is contemplated that in certain embodiments the same principles apply to the use of the disclosed low non-specific binding supports and nucleic acid hybridization and amplification formulations for other detection modes as well, including both optical and non-optical detection modes.

In some embodiments, the disclosed low-binding supports, optionally used in combination with the disclosed hybridization and/or amplification protocols, yield solid-phase reactions that exhibit: (i) negligible non-specific binding of protein and other reaction components (thus minimizing substrate background), (ii) negligible non-specific nucleic acid amplification product, and (iii) provide tunable nucleic acid amplification reactions.

In some embodiments, fluorescence images of the disclosed low background surfaces when used in nucleic acid hybridization or amplification applications to create polonies of hybridized or clonally-amplified nucleic acid molecules (e.g., that have been directly or indirectly labeled with a fluorophore) exhibit contrast-to-noise ratios (CNRs) of at least 10, 20, 30, 40, 50, 60, 70, 80, 90, 100, 110, 120, 130, 140, 150, 160, 170, 180, 190, 20, 210, 220, 230, 240, 250, or greater than 250.

In some embodiments, a fluorescence image of the surface exhibits a contrast-to-noise ratio (CNR) of at least 20 when a sample nucleic acid molecule or complementary sequences thereof are labeled with a Cyanine dye-3 (Cy3) fluorophore, and when the fluorescence image is acquired using an inverted fluorescence microscope (e.g., Olympus IX83) with a 20×0.75 NA objective, a 532 nm light source, a bandpass and dichroic mirror filter set optimized for 532 nm excitation and Cy3 fluorescence emission, and a camera (e.g., Andor sCMOS, Zyla 4.2) under non-signal saturating conditions while the surface is immersed in a buffer (e.g., 25 mM ACES, pH 7.4 buffer).

Throughout this application, various publications, patents, and/or patent applications are referenced. The disclosures of the publications, patents and/or patent applications are hereby incorporated by reference in their entireties into this application in order to more fully describe the state of the art to which this disclosure pertains.

### EXAMPLES

The following examples are meant to be illustrative and can be used to further understand embodiments of the present disclosure and should not be construed as limiting the scope of the present teachings in any way.

### Example 1: Preparation of USER™-Treated Linear Nucleic Acid Libraries

Commercially available kits were used to prepare linear nucleic acid libraries having a sequence of interest (insert) appended to at least one universal sequencing primer, and universal binding sequences P5 (e.g., first surface primer) and P7 (e.g., second surface primer) at their terminal ends. The commercially available kits are intended for preparing library molecules for a next generation sequencing platform (such as Illumina™). Input nucleic acids containing the sequences of interest included DNA from E. coli. The input DNA was sheared using Covaris™ to achieve insert sizes of about 200-400 bp according to the manufacturer's instructions. The amount of input fragmented nucleic acids used to prepare the linear library molecules included 50 ng, 100 ng, 500 ng and 1 μg. The linear library molecules were treated with USER™ enzyme as follows. Fragmented E. coli DNA (30 μL) was mixed with water (14 μL), 10×CutSmart™ Buffer (5 μL), thermolabile USER II™ (1 μL). The reaction was incubated at 37 degrees C. for 15 minutes. The USER™-treated library was cleaned-up with 2×SPRI SELECT™ beads (from Beckman Coulter™) (108 μL), incubated, washed twice with 80% ethanol, dried, and eluted in 32 μL of aqueous buffer.

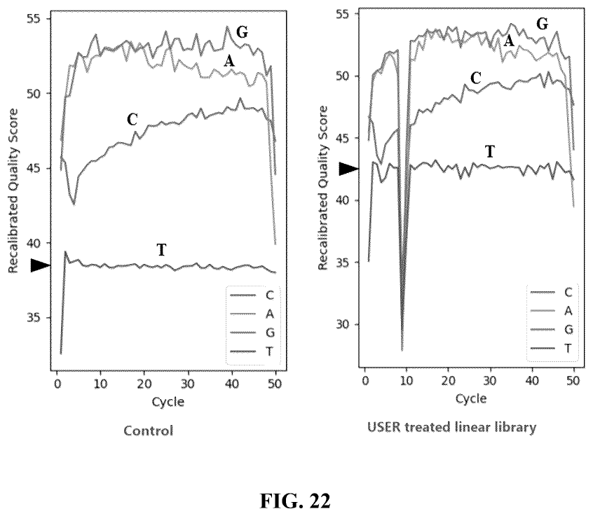

The USER™-treated library was circularized using double-stranded splint adaptors (e.g., see FIG. 1 and FIG. 2, and shown in Examples 2-3 below) to generate covalently closed circular library molecules that were distributed onto a support having immobilized capture primers, and subjected to rolling circle amplification to generate immobilized concatemer template molecules for sequencing. Treatment was prior to hybridization to the double-stranded splint adaptors. A graph of the sequencing quality scores is shown in FIG. 22 (right-hand graph). The control graph shows that the quality scores of the T base calls were approximately 39. The USER™-treated graph on the right shows the sequencing quality scores of base calls T, C, A and G of concatemer template molecules that generated with USER™ treatment during the library prep workflow. The USER™-treated graph shows that the quality scores of the T base calls increased to approximately 43.

### Example 2: Preparing Library-Splint Complexes

Linear library preps (e.g., 0.25, 0.5 or 1 μmol) were annealed to the double-stranded splint adaptors (200) in an annealing buffer containing 100 mM potassium acetate and 30 mM HEPES (pH 7.5), in a thermal cycler apparatus. The annealing program included: 5 minutes at degrees C., 5 minutes at 37 degrees C., and hold at 37 degrees C.

The double-stranded splint adaptors comprised: first splint strands (300) hybridized to second splint strands (400). An exemplary double-stranded splint adaptor is shown in FIG. 1.

The second splint strands (400) comprised: a first sub-region that hybridizes with a third surface primer and a second sub-region that hybridizes with a fourth surface primer (or a complementary sequence thereof). The 5′ ends of the second splint strands (400) carried a phosphorylated end. The second splint strands (400) are designed to carry universal sequences that are not found in a commercially available library preparation kit.

The first splint strands (300) comprised: a first region (320) that hybridizes with a P5 sequence at one end of the linear library molecule; an internal region (310) which hybridized with the second splint strands (400); and a second region (330) that hybridizes with a P7 sequence at the other end of the linear library molecule.

### Example 3: Preparing Covalently Closed Circular Library Molecules

The annealing mixture from Example 2 was subjected to an enzymatic ligation and phosphorylation reaction by adding to the annealing mixture T7 DNA ligase and T4 polynucleotide kinase with a T4 DNA ligase reaction buffer. The enzymatic mixture was incubated in a thermal cycling apparatus with a heated lid set to 75 degrees C. The thermal cycling apparatus program included: 10 minutes at 37 degrees, 10 minutes at 65 degrees, and hold at 4 degrees. The ligation and phosphorylation reactions generated covalently closed circular library molecules (600) that were hybridized to first splint strands (300).

An enzymatic exonuclease digestion was conducted by adding to the ligation/phosphorylation reaction mixture T7 exonuclease and Thermolabile exonuclease. The exonuclease reaction mixture was incubated in a thermal cycling apparatus which was programed: 10 minutes at 37 degrees C., 2 minutes at 80 degrees C., and hold at 4 degrees C.

The exonuclease reaction mixture was subjected to multiple cycles of clean-up using SPRI SELECT™ beads (from Beckman Coulter™) according to the manufacturer's instructions.

The yield of the cleaned preparation of covalently closed circular library molecules (e.g., single stranded molecules) were quantified using Qubit™ or qPCR.

### Example 4: Preparing USER™-Treated Covalently Closed Circular Library Molecules

In a separate experiment, a linear library was prepared as described in Example 1 but the linear library was not treated with USER™. The linear library was used to generate library-splint complexes as described in Example 2. The library-splint complexes were subjected to a ligation reaction to generate covalently closed circular molecules as described in Example 3 through the enzymatic exonuclease digestion step using T7 exonuclease and Thermolabile exonuclease. The covalently closed circular molecules were treated with USER™ as follows: thermolabile USER™ (2 μL) was added to the covalently closed circular library molecules and incubated as 37 degrees for 10 minutes, and heat-killed at 80 degrees for 2 minutes. The USER™-treated circular molecules were subjected to multiple cycles of clean-up using SPRI SELECT™ beads (from Beckman Coulter™) according to the manufacturer's instructions.

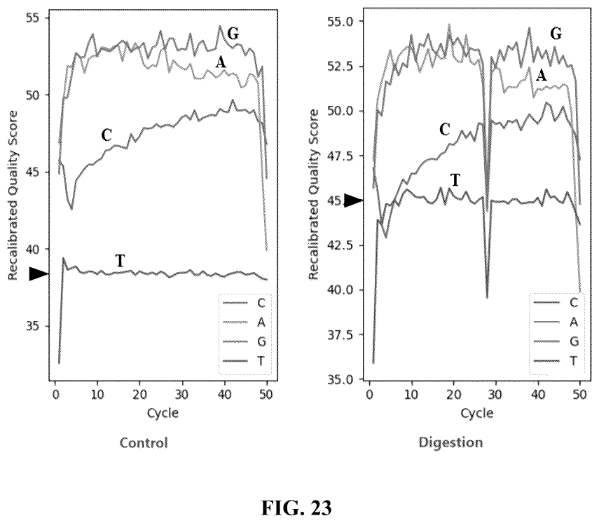

The USER™-treated circularized library molecules were distributed onto a support having immobilized capture primers and subjected to rolling circle amplification to generate immobilized concatemer template molecules for sequencing. The linear library molecules were treated after enzymatic digestion of the first splint strand (300) with T7 exonuclease and Thermolabile exonuclease. A graph of the sequencing quality scores is shown in FIG. 23 (right-hand graph). The control graph shows that the quality scores of the T base calls were approximately 39. The USER™-treated graph on the right shows the sequencing quality scores of base calls T, C, A and G of concatemer template molecules that generated with USER™ treatment during the library prep workflow. The USER™-treated graph shows that the quality scores of the T base calls increased to approximately 45.

### Example 5: Rolling Circle Amplification and Sequencing

The covalently closed circular library molecules from Examples 3 or 4 were distributed onto a support that was passivated with a low non-specific binding coating in the presence of a high efficiency hybridization buffer and subjected to on-support rolling circle amplification to generate immobilized concatemers.

### Example 6: Sequencing Using Multivalent Molecules and Nucleotides

The concatemers were subjected to recursive two-stage sequencing reactions using fluorescently labeled multivalent molecules in the first stage and un-labeled nucleotide analogs (e.g., 3′ chain terminator blocking group) in the second stage.

The two-stage sequencing reaction was conducted on a flow cell having a plurality of concatemer template molecules immobilized thereon (e.g., immobilized polonies).

The first-stage sequencing reaction was conducted by hybridizing a plurality of a soluble sequencing primers to concatemer template molecules that were immobilized to a flow cell to form immobilized primer-concatemer duplexes. A plurality of a first sequencing polymerase was flowed onto the flow cell (e.g., contacting the immobilized primer-concatemer duplexes) and incubated under a condition suitable to bind the sequencing polymerase to the duplexes to form complexed polymerases. A mixture of fluorescently labeled multivalent molecules (e.g., at different concentrations of about 20-100 nM) was flowed onto the flow cell in the presence of a buffer that included a non-catalytic cation (e.g., strontium, barium and/or calcium) and incubated under conditions suitable to bind complementary nucleotide units of the multivalent molecules to the complexed polymerases to form avidity complexes without polymerase-catalyzed incorporation of the nucleotide units. The fluorescently labeled multivalent molecules were labeled at their cores. The complexed polymerases were washed. An image was obtained of the fluorescently labeled multivalent molecules that remained bound to the complexed polymerases. The first sequencing polymerases and multivalent molecules were removed, while retaining the sequencing primers hybridized to the immobilized concatemers (retained duplexes), by washing with a buffer comprising a detergent.

The first stage sequencing reaction was suitable for forming a plurality of avidity complexes on the concatemer template molecules (e.g., polonies). For example, the first stage sequencing reaction comprised: (a) binding a first nucleic acid primer, a first polymerase, and a first multivalent molecule to a first portion of a concatemer template molecule thereby forming a first binding complex, wherein a first nucleotide unit of the first multivalent molecule was bound to the first polymerase; and (b) binding a second nucleic acid primer, a second polymerase, and the first multivalent molecule to a second portion of the same concatemer template molecule thereby forming a second binding complex, wherein a second nucleotide unit of the first multivalent molecule was bound to the second polymerase, wherein the first and second binding complexes which included the same multivalent molecule formed a first avidity complex.

The second-stage sequencing reaction was conducted by contacting the retained duplexes with a plurality of second sequencing polymerases to form complexed polymerases. A mixture of non-labeled nucleotide analogs (e.g., 3′O-methylazido nucleotides) (e.g., at different concentrations of about 1-5 μM) was added to the complexed polymerases in the presence of a buffer that included a catalytic cation (e.g., magnesium and/or manganese) and incubated under conditions suitable to bind complementary nucleotides to the complexed polymerases and promote polymerase-catalyzed incorporation of the nucleotides to generate a nascent extended sequencing primer. The complexed polymerases were washed. No image was obtained. The incorporated non-labeled nucleotide analogs were reacted with a cleaving reagent that removes the 3′ O-methylazido group and generates an extendible 3′OH group.

In an alternative second stage sequencing reaction, a mixture of fluorescently labeled nucleotide analogs (e.g., 3′O-methylazido nucleotides) (e.g., about 1-5 μM) was added to the complexed polymerases in the presence of a buffer that included a catalytic cation (e.g., magnesium and/or manganese) and incubated under conditions suitable to bind complementary nucleotides to the complexed polymerases and promote polymerase-catalyzed incorporation of the nucleotides to generate a nascent extended sequencing primer. The complexed polymerases were washed. An image was obtained of the incorporated fluorescently labeled nucleotide analogs as a part of the complexed polymerases. The incorporated fluorescently labeled nucleotide analogs were reacted with a cleaving reagent that removes the 3′ O-methylazido group and generates an extendible 3′OH group.

The second sequencing polymerases were removed, while retaining the nascent extended sequencing primers hybridized to the concatemers (retained duplexes), by washing with a buffer comprising a detergent. Recurring sequencing reactions were conducted by performing multiple cycles of first stage and second-stage sequencing reactions to generate extended forward sequencing primer strands.

### INCORPORATION BY REFERENCE

All publications and patent applications mentioned in this specification are herein incorporated by reference to the same extent as if each individual publication or patent application was specifically and individually indicated to be incorporated by reference.

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

What is claimed is:

1. A method for reducing deaminated nucleotide bases in a nucleic acid library, comprising:
a) providing a plurality of linear nucleic acid library molecules, wherein individual library molecules in the plurality comprise a sequence of interest joined to at least one universal adaptor sequence having a binding sequence for a surface capture primer and one universal adaptor sequence having a binding sequence for a sequencing primer, and wherein at least one of the library molecule carries one or more deaminated nucleotide bases;
b) contacting the plurality of nucleic acid library molecules with a reagent that removes deaminated nucleotide bases, thereby generating at least one library molecule carrying an abasic site;
c) circularizing the plurality of nucleic acid library molecules to generate a plurality of covalently closed circular library molecules;
d) distributing the plurality of covalently closed circular library molecules onto a support having a plurality of immobilized surface capture primers, under a condition suitable for hybridizing individual covalently closed circular library molecules to a surface capture primer;
e) conducting a rolling circle amplification reaction to generate a plurality of nucleic acid concatemer template molecules immobilized to the support; and
f) sequencing the plurality of nucleic acid concatemer template molecules to determine the sequence of at least a portion of the concatemer template molecules.

2. The method of claim 1, further comprising: (g) contacting the plurality of covalently closed circular library molecules with a reagent the removes deaminated nucleotide bases, thereby generating at least one circular library molecule carrying an abasic site.

3. The method of claim 1, wherein the reagent that removes deaminated nucleotide bases of step (b) comprises DNA glycosylase (UDG) and (i) AP lyase, (ii) Endo IV endonuclease, (iii) FPG glycosylase/AP lyase, and/or (iv) Endo VIII glycosylase/AP lyase, or a combination thereof.

4. The method of claim 1, wherein the plurality of immobilized surface capture primers is tethered to a polymer coating on the support.

5. The method of claim 1, wherein the rolling circle amplification reaction of step (e) comprises a strand displacing polymerase and a plurality of nucleotides comprising dATP, dGTP, dCTP, dTTP and/or dUTP.

6. The method of claim 1, wherein the plurality of immobilized surface capture primers is located at pre-determined locations on the support.

7. The method of claim 1, wherein the plurality of immobilized surface capture primers is located at random locations on the support.

8. The method of claim 1, wherein the plurality of immobilized concatemer template molecules on the support is in fluid communication with each other to permit flowing a solution of reagents onto the support.

9. The method of claim 8, wherein the solution of reagents comprises enzymes, nucleotides, and divalent cations.

10. The method of claim 8, wherein the plurality of immobilized concatemer template molecules is essentially simultaneously reacted with the reagents in a massively parallel manner.

11. The method of claim 1, wherein the density of the plurality of immobilized concatemer template molecules on the polymer-coated support is 10²-10¹² per mm².

12. The method of claim 1, wherein sequencing the plurality of immobilized concatemers comprises:
a) contacting the plurality of immobilized concatemer molecules with (i) a plurality of sequencing polymerases and (ii) a plurality of soluble sequencing primers, wherein the contacting is conducted under a condition suitable to form a plurality of complexed polymerases each comprising a sequencing polymerase bound to a nucleic acid duplex, wherein the nucleic acid duplex comprises a concatemer molecule hybridized to a soluble sequencing primer;
b) contacting the plurality of complexed sequencing polymerases with a plurality of nucleotides under a condition suitable for binding at least one nucleotide to a complexed sequencing polymerase, wherein the plurality of nucleotides comprises at least one nucleotide analog labeled with a fluorophore and having a removable chain terminating moiety at the sugar 3′ position;
c) incorporating at least one nucleotide into the 3′ end of the hybridized sequencing primers, thereby generating a plurality of nascent extended sequencing primers; and
d) detecting the incorporated nucleotide and identifying the nucleo-base of the incorporated nucleotide.

13. The method of claim 12, wherein the plurality of nucleotides comprises:
a) a removable chain terminating moiety at the 3′ sugar group, wherein the removable chain terminating moiety comprises an alkyl group, alkenyl group, alkynyl group, allyl group, aryl group, benzyl group, azide group, azido group, O-azidomethyl group, amine group, amide group, keto group, isocyanate group, phosphate group, thio group, disulfide group, carbonate group, urea group, or silyl group;
b) a removable chain terminating moiety is cleavable with a chemical compound to generate an extendible 3′OH moiety on the sugar group; and/or
c) one type of nucleotide or a mixture of any combination of two or more types of nucleotides selected from the group consisting of dATP, dGTP, dCTP, dTTP and dUTP.

14. The method of claim 13, wherein sequencing the plurality of immobilized concatemers comprises:
a) contacting the plurality of immobilized concatemer molecules with (i) a plurality of sequencing polymerases and (ii) a plurality of the soluble sequencing primers, wherein the contacting is conducted under a condition suitable to form a plurality of first complexed polymerases each comprising a sequencing polymerase bound to a nucleic acid duplex, wherein the nucleic acid duplex comprises a concatemer molecule hybridized to a soluble sequencing primer;
b) contacting the plurality of complexed sequencing polymerases with a plurality of detectably labeled multivalent molecules to form a plurality of multivalent-complexed polymerases, under a condition suitable for binding complementary nucleotide units of the multivalent molecules to at least two of the plurality of first complexed polymerases thereby forming a plurality of multivalent-complexed polymerases, and the condition inhibits incorporation of the complementary nucleotide units into the sequencing primers of the plurality of multivalent-complexed polymerases, wherein individual multivalent molecules in the plurality of multivalent molecules comprise a core attached to multiple nucleotide arms and each nucleotide arm is attached to a nucleotide unit;
c) detecting the plurality of multivalent-complexed polymerases; and
d) identifying the nucleo-base of the complementary nucleotide units that are bound to the plurality of first complexed polymerases in the plurality of multivalent-complexed polymerases, thereby determining the sequence of the nucleic acid template.

15. The method of claim 14, further comprising:
e) dissociating the plurality of multivalent-complexed polymerases, removing the plurality of first sequencing polymerases and their bound multivalent molecules, and retaining the plurality of nucleic acid duplexes;
f) contacting the plurality of the retained nucleic acid duplexes of step (e) with a plurality of second sequencing polymerases, wherein the contacting is conducted under a condition suitable for binding the plurality of second sequencing polymerases to the plurality of the retained nucleic acid duplexes, thereby forming a plurality of second complexed polymerases each comprising a second sequencing polymerase bound to a retained nucleic acid duplex;
g) contacting the plurality of second complexed polymerases with a plurality of nucleotides comprising at least one nucleotide analog labeled with a fluorophore and having a removable chain terminating moiety at the sugar 3′ position, wherein the contacting is conducted under a condition suitable for binding complementary nucleotides from the plurality of nucleotides to at least two of the second complexed polymerases of step (0 thereby forming a plurality of nucleotide-complexed polymerases and the condition is suitable for promoting incorporation of the bound complementary nucleotides into the sequencing primers of the nucleotide-complexed polymerases.

16. The method of claim 15, further comprising:
h) detecting the complementary nucleotides which are incorporated into the sequencing primers of the nucleotide-complexed polymerases; or
h) detecting the complementary nucleotides which are incorporated into the sequencing primers of the nucleotide-complexed polymerases; and
i) identifying the nucleo-bases of the complementary nucleotides which are incorporated into the sequencing primers of the nucleotide-complexed polymerases.

17. The method of claim 1, wherein:
i. the support comprises a glass substrate;
ii. the support comprises a plastic substrate; and/or
iii. the support is passivated with at least one hydrophilic polymer coating having a water contact angle of no more than 45 degrees and selected from the group consisting of polyethylene glycol (PEG), poly(vinyl alcohol) (PVA), poly(vinyl pyridine), poly(vinyl pyrrolidone) (PVP), poly(acrylic acid) (PAA), polyacrylamide, poly(N-isopropylacrylamide) (PNIPAM), poly(methyl methacrylate) (PMA), poly(2-hydroxylethyl methacrylate) (PHEMA), poly(oligo(ethylene glycol) methyl ether methacrylate) (POEGMA), polyglutamic acid (PGA), poly-lysine, poly-glucoside, streptavidin, and dextran.

18. The method of claim 1, further comprising:
i. determining the percent base call error from the sequencing of step (f); and/or
ii. determining the Phred quality score of the sequencing data from the percent base call error.

19. A method for sequencing by forming at least one avidity complex, comprising:
a) generating a nucleic acid concatemer by conducting a rolling circle amplification on a closed circular nucleic acid molecule comprising at least one abasic site, wherein the abasic site is generated by contacting a closed circular nucleic acid molecule or the corresponding linear nucleic acid molecule with a reagent that removes deaminated nucleotide bases;
b) binding a first universal sequencing primer, a first sequencing polymerase, and a first detectably labeled multivalent molecule to a first portion of the concatemer molecule, thereby forming a first binding complex, wherein a first nucleotide unit of the first multivalent molecule binds to the first sequencing polymerase;
c) binding a second universal sequencing primer, a second sequencing polymerase, and the first detectably labeled multivalent molecule to a second portion of the same concatemer molecule thereby forming a second binding complex, wherein a second nucleotide unit of the first multivalent molecule binds to the second sequencing polymerase, wherein the first and second binding complexes which include the same multivalent molecule forms an avidity complex, wherein the first detectably labeled multivalent molecule comprises a core attached to multiple nucleotide arms and each nucleotide arm is attached to a nucleotide unit, wherein the concatemer molecule comprises two or more tandem repeat sequences of a sequence of interest (110) and a universal primer binding site that binds the first and second universal sequencing primers, and wherein the contacting is conducted under a condition suitable to inhibit polymerase-catalyzed incorporation of the bound first and second nucleotide units in the first and second binding complexes;
d) detecting the first and second binding complexes on the same concatemer molecule, and
e) identifying the first nucleotide unit in the first binding complex thereby determining the sequence of the first portion of the concatemer template molecule, and identifying the second nucleotide unit in the second binding complex thereby determining the sequence of the second portion of the concatemer template molecule.

20. The method of claim 19, wherein:
i. the plurality of nucleotide arms attached to the core of the individual multivalent molecules has the same type of nucleotide unit, and wherein the type of nucleotide unit is selected from the group consisting of dATP, dGTP, dCTP, dTTP and dUTP;
ii. the plurality of multivalent molecules comprises a mixture of two or more types of multivalent molecules, each type having a nucleotide unit selected from the group consisting of dATP, dGTP, dCTP, dTTP and dUTP;
iii. the plurality of nucleotides comprises a removable chain terminating moiety at the 3′ sugar group, wherein the removable chain terminating moiety comprises an alkyl group, alkenyl group, alkynyl group, allyl group, aryl group, benzyl group, azide group, azido group, O-azidomethyl group, amine group, amide group, keto group, isocyanate group, phosphate group, thio group, disulfide group, carbonate group, urea group, or silyl group, wherein the removable chain terminating moiety is cleavable with a chemical compound to generate an extendible 3′OH moiety on the sugar group; and/or
iv. the plurality of nucleotides comprises one type of nucleotide or a mixture of any combination of two or more types of nucleotides selected from the group consisting of dATP, dGTP, dCTP, dTTP and dUTP.


SEQUENCE LISTING (214 sequences)

SEQ ID NO: 1 - DNA, 25 bp, synthetic construct
  source 1..25: mol_type = other DNA
catgtaatgcacgtactttcagggt

SEQ ID NO: 2 - DNA, 23 bp, synthetic construct
  source 1..23: mol_type = other DNA
agtcgtcgcagcctcacctgatc

SEQ ID NO: 3 - DNA, 48 bp, synthetic construct
  source 1..48: mol_type = other DNA
agtcgtcgcagcctcacctgatccatgtaatgcacgtactttcagggt

SEQ ID NO: 4 - DNA, 20 bp, synthetic construct
  source 1..20: mol_type = other DNA
tcggtggtcgccgtatcatt

SEQ ID NO: 5 - DNA, 21 bp, synthetic construct
  source 1..21: mol_type = other DNA
caagcagaagacggcatacga

SEQ ID NO: 6 - DNA, 25 bp, synthetic construct
  source 1..25: mol_type = other DNA
accctgaaagtacgtgcattacatg

SEQ ID NO: 7 - DNA, 23 bp, synthetic construct
  source 1..23: mol_type = other DNA
gatcaggtgaggctgcgacgact

SEQ ID NO: 8 - DNA, 89 bp, synthetic construct
  source 1..89: mol_type = other DNA
tcggtggtcgccgtatcattaccctgaaagtacgtgcattacatggatcaggtgaggctgcgacgactcaagcagaagacggcatacga

SEQ ID NO: 9 - DNA, 25 bp, synthetic construct
  source 1..25: mol_type = other DNA
accctgaaagtacgtgcattacatg

SEQ ID NO: 10 - DNA, 23 bp, synthetic construct
  source 1..23: mol_type = other DNA
gatcaggtgaggctgcgacgact

SEQ ID NO: 11-202 - skipped (192 entries)

SEQ ID NO: 203 - DNA, 48 bp, synthetic construct
  source 1..48: mol_type = other DNA
accctgaaagtacgtcgattacatggatcaggtgaggctgcgacgact

SEQ ID NO: 204 - DNA, 20 bp, synthetic construct
  source 1..20: mol_type = other DNA
aatgatacggcgaccaccga

SEQ ID NO: 205 - DNA, 24 bp, synthetic construct
  source 1..24: mol_type = other DNA
aatgatacggcgaccaccgagatc

SEQ ID NO: 206 - DNA, 24 bp, synthetic construct
  source 1..24: mol_type = other DNA
caagcagaagacggcatacgagat

SEQ ID NO: 207 - DNA, 33 bp, synthetic construct
  source 1..33: mol_type = other DNA
acactctttccctacacgacgctcttccgatct

SEQ ID NO: 208 - DNA, 33 bp, synthetic construct
  source 1..33: mol_type = other DNA
tcgtcggcagcgtcagatgtgtataagagacag

SEQ ID NO: 209 - DNA, 35 bp, synthetic construct
  source 1..35: mol_type = other DNA
cgtgctggattggctcaccagacaccttccgacat

SEQ ID NO: 210 - DNA, 34 bp, synthetic construct
  source 1..34: mol_type = other DNA
agatcggaagagcacacgtctgaactccagtcac

SEQ ID NO: 211 - DNA, 34 bp, synthetic construct
  source 1..34: mol_type = other DNA
ctgtctcttatacacatctccgagcccacgagac

SEQ ID NO: 212 - DNA, 35 bp, synthetic construct
  source 1..35: mol_type = other DNA
atgtcggaaggtgtgcaggctaccgcttgtcaact

SEQ ID NO: 213 - DNA, 21 bp, synthetic construct
  source 1..21: mol_type = other DNA
tcgtatgccgtcttctgcttg

SEQ ID NO: 214 - DNA, 41 bp, synthetic construct
  source 1..41: mol_type = other DNA
tcggtggtcgccgtatcattcaagcagaagacggcatacga